SEQUENCE LISTING (32 sequences)

SEQ ID NO: 1 - DNA, 19 bp, synthetic construct
  misc_feature 1..19: note = CDH1 Forward
  source 1..19: mol_type = other DNA
gaccggtgcaatcttcaaa

SEQ ID NO: 2 - DNA, 19 bp, synthetic construct
  misc_feature 1..19: note = CDH1 Reverse
  source 1..19: mol_type = other DNA
ttgacgccgagagctacac

SEQ ID NO: 3 - DNA, 20 bp, synthetic construct
  misc_feature 1..20: note = CHGA Forward
  source 1..20: mol_type = other DNA
tgtgtcggagatgacctcaa

SEQ ID NO: 4 - DNA, 20 bp, synthetic construct
  misc_feature 1..20: note = CHGA Reverse
  source 1..20: mol_type = other DNA
gtcctggctcttctgctctg

SEQ ID NO: 5 - DNA, 20 bp, synthetic construct
  misc_feature 1..20: note = CKB Forward
  source 1..20: mol_type = other DNA
cccacaccaggaaggtctta

SEQ ID NO: 6 - DNA, 18 bp, synthetic construct
  misc_feature 1..18: note = CKB Reverse
  source 1..18: mol_type = other DNA
cctcttcgacaagcccgt

SEQ ID NO: 7 - DNA, 20 bp, synthetic construct
  misc_feature 1..20: note = FXYD3 Forward
  source 1..20: mol_type = other DNA
agggtcaccttctgcatgtc

SEQ ID NO: 8 - DNA, 20 bp, synthetic construct
  misc_feature 1..20: note = FXYD3 Reverse
  source 1..20: mol_type = other DNA
cttcggataaacgcaggact

SEQ ID NO: 9 - DNA, 20 bp, synthetic construct
  misc_feature 1..20: note = GATA4 Forward
  source 1..20: mol_type = other DNA
tagccccacagttgacacac

SEQ ID NO: 10 - DNA, 17 bp, synthetic construct
  misc_feature 1..17: note = GATA4 Reverse
  source 1..17: mol_type = other DNA
gtcctgcacagcctgcc

SEQ ID NO: 11 - DNA, 20 bp, synthetic construct
  misc_feature 1..20: note = HOXA13 Forward
  source 1..20: mol_type = other DNA
gcaccttggtataaggcacg

SEQ ID NO: 12 - DNA, 20 bp, synthetic construct
  misc_feature 1..20: note = HOXA13 Reverse
  source 1..20: mol_type = other DNA
cctctggaagtccactctgc

SEQ ID NO: 13 - DNA, 20 bp, synthetic construct
  misc_feature 1..20: note = HOXB13 Forward
  source 1..20: mol_type = other DNA
gctgtacggaatgcgtttct

SEQ ID NO: 14 - DNA, 19 bp, synthetic construct
  misc_feature 1..19: note = HOXB13 Reverse
  source 1..19: mol_type = other DNA
aacccaccaggtccctttt

SEQ ID NO: 15 - DNA, 20 bp, synthetic construct
  misc_feature 1..20: note = HOXD13 Forward
  source 1..20: mol_type = other DNA
cctcttcggtagacgcacat

SEQ ID NO: 16 - DNA, 20 bp, synthetic construct
  misc_feature 1..20: note = HOXD13 Reverse
  source 1..20: mol_type = other DNA
caggtgtactgcaccaagga

SEQ ID NO: 17 - DNA, 20 bp, synthetic construct
  misc_feature 1..20: note = HOXD3 Forward
  source 1..20: mol_type = other DNA
cacctccaatgtctgctgaa

SEQ ID NO: 18 - DNA, 23 bp, synthetic construct
  misc_feature 1..23: note = HOXD3 Reverse
  source 1..23: mol_type = other DNA
caaaattcaagaaaacacacaca

SEQ ID NO: 19 - DNA, 19 bp, synthetic construct
  misc_feature 1..19: note = INSL5 Forward
  source 1..19: mol_type = other DNA
gaaggttttgcgctggatt

SEQ ID NO: 20 - DNA, 20 bp, synthetic construct
  misc_feature 1..20: note = INSL5 Reverse
  source 1..20: mol_type = other DNA
gatccctcaagctcagcaag

SEQ ID NO: 21 - DNA, 20 bp, synthetic construct
  misc_feature 1..20: note = MSX2 Forward
  source 1..20: mol_type = other DNA
ggtcttgtgtttcctcaggg

SEQ ID NO: 22 - DNA, 20 bp, synthetic construct
  misc_feature 1..20: note = MSX2 Reverse
  source 1..20: mol_type = other DNA
aaattcagaagatggagcgg

SEQ ID NO: 23 - DNA, 20 bp, synthetic construct
  misc_feature 1..20: note = MUC2 Forward
  source 1..20: mol_type = other DNA
tgtaggcatcgctcttctca

SEQ ID NO: 24 - DNA, 20 bp, synthetic construct
  misc_feature 1..20: note = MUC2 Reverse
  source 1..20: mol_type = other DNA
gacaccatctacctcacccg

SEQ ID NO: 25 - DNA, 20 bp, synthetic construct
  misc_feature 1..20: note = ONECUT1 Forward
  source 1..20: mol_type = other DNA
tttttgggtgtgttgcctct

SEQ ID NO: 26 - DNA, 19 bp, synthetic construct
  misc_feature 1..19: note = ONECUT1 Reverse
  source 1..19: mol_type = other DNA
agaccttccggaggatgtg

SEQ ID NO: 27 - DNA, 18 bp, synthetic construct
  misc_feature 1..18: note = PDX1 Forward
  source 1..18: mol_type = other DNA
cgtccgcttgttctcctc

SEQ ID NO: 28 - DNA, 20 bp, synthetic construct
  misc_feature 1..20: note = PDX1 Reverse
  source 1..20: mol_type = other DNA
cctttcccatggatgaagtc

SEQ ID NO: 29 - DNA, 21 bp, synthetic construct
  misc_feature 1..21: note = PPIA (CPHA) Forward
  source 1..21: mol_type = other DNA
cccaccgtgttcttcgacatt

SEQ ID NO: 30 - DNA, 22 bp, synthetic construct
  misc_feature 1..22: note = PPIA (CPHA) Reverse
  source 1..22: mol_type = other DNA
ggacccgtatgctttaggatga

SEQ ID NO: 31 - DNA, 19 bp, synthetic construct
  misc_feature 1..19: note = SATB2 Forward
  source 1..19: mol_type = other DNA
ccaccttcccagcttgatt

SEQ ID NO: 32 - DNA, 20 bp, synthetic construct
  misc_feature 1..20: note = SATB2 Reverse
  source 1..20: mol_type = other DNA
ttagccagctggtggagact

(19) United States
(12) Patent Application Publication
(10) Pub. No.: US 2024/0240154 A1
(43) Pub. Date: Jul. 18, 2024

(54) COLONIC ORGANOIDS AND METHODS OF MAKING AND USING SAME

(71) Applicant: Children's Hospital Medical Center, Cincinnati, OH (US)

(72) Inventors: James M. Wells, Cincinnati, OH (US); Jorge Orlando Munera, Cincinnati, OH (US)

(21) Appl. No.: 18/473,619

(22) Filed: Sep. 25, 2023

Related U.S. Application Data
(63) Continuation of application No. 16/461,147, filed on May 15, 2019, now Pat. No. 11,767,515.
(60) Provisional application No. 62/478,962, filed on Mar. 30, 2017.
(60) Provisional application No. 62/429,948, filed on Dec. 5, 2016.

(51) Int. Cl. C12N 5/071

(52) U.S. Cl. CPC C12N 5/0679; C12N 2501/11; C12N 2501/113; C12N 2501/115; C12N 2501/117; C12N 2501/119; C12N 2501/155; C12N 2501/415; C12N 2506/02

(57) ABSTRACT

Disclosed herein are methods for the in vitro differentiation of a precursor cell into definitive endoderm, which may further be differentiated into a human colonic organoid (HCO), via modulation of signaling pathways. Further disclosed are HCOs and methods of using HCOs, which may be used, for example, for the HCOs may be used to determine the efficacy and/or toxicity of a potential therapeutic agent for a disease selected from colitis, colon cancer, polyposis syndromes, and/or irritable bowel syndrome.

### CROSS REFERENCE TO RELATED APPLICATIONS

This application claims priority to and the benefit of PCT Application No. PCT/US2017/064600 filed Dec. 5, 2017, U.S. Provisional Application Ser. No. 62/478,962 filed Mar. 30, 2017, and U.S. Provisional Application Ser. No. 62/429,948 filed Dec. 5, 2016, each of which is incorporated herein by reference it its entirety for all purposes.

### STATEMENT REGARDING FEDERALLY SPONSORED R&D

This invention was made with government support under EB021780, DK103117, and AI116491 awarded by the National Institutes of Health. The government has certain rights in the invention.

### BACKGROUND

While the generation of gastric and small intestinal organoids from pluripotent stem cells (PSCs) has revolutionized the study of human gastrointestinal (GI) development and disease, the efforts to generate large intestinal organoids have lagged behind, in part due to the lack of a robust understanding of posterior gut tube development.

### BRIEF SUMMARY

Disclosed herein are methods for the in vitro differentiation of a precursor cell into definitive endoderm, which may further be differentiated into a human colonic organoid (HCO), via modulation of signaling pathways. Further disclosed are HCOs and methods of using HCOs, which may be used, for example, for the HCOs may be used to determine the efficacy and/or toxicity of a potential therapeutic agent for a disease selected from colitis, colon cancer, polyposis syndromes, and/or irritable bowel syndrome.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIGS. 1A-V. Bmp signaling regulates Satb2 expression in mouse and frog embryos. (FIG. 1A) Whole-mount pSmad158 (red) and Foxa2 (green) staining of e8.5 mouse embryo showing nuclear staining around the developing hindgut (n=6). (FIG. 1B) Inset of optical slices from boxed region in (FIG. 1A) showing pSmad 1/5/8 staining in the hindgut mesoderm and endoderm (D, dorsal; V, ventral). (FIG. 1C) Schematic of mouse embryo isolated at the headfold stage and cultured for 2 days+/−Bmp inhibition with DMH-1. (FIGS. 1D,1E) Whole-mount pSmad1/5/8 (red) and Foxa2 (green) staining of DMSO (FIG. 1D) and DMH-1 (FIG. 1E) treated embryos after 48 hours of culture. (FIG. 1F) Quantification of pSmad1/5/8 and pSmad2/3 staining in relative to Cdx2 in embryos cultured in DMSO or DMH-1 (n=3 embryos per condition). (FIGS. 1G-J) Whole-mount immunostaining of Cdx2 (green), Satb2 (red) and Foxa2 (white) of mouse embryos (n=6 for each condition) following 2 days of culture in DMSO (FIGS. 1G,1H) or DMH-1 (FIGS. 11, 1J). Arrows in FIGS. 1H-1J point to the approximate location of the yolk stalk (BA1, first brachial arch). (FIG. 1K) Quantification of Satb2 expression in mouse embryos treated with DMSO or DMH-1. (FIG. 1L) Schematic of Bmp inhibition in Xenopus tropicalis embryos. In situ hybridization of Satb2 in Xenopus tropicalis embryos treated with DMSO (FIG. 1M) or DMH-1 (FIG. 1R). The white dotted line in (FIG. 1M) and (FIG. 1R) depict the plane of section used subsequent analysis. Mx and md=maxillary and mandibular processes of first brachial arch. Cba=Caudal brachial arches. Immunofluorescence of Satb2 (red), pSmad1/5/8 (green), DAPI (blue), and color merged images from Xenopus tropicalis embryos treated with DMSO (FIGS. 1N-Q) or DMH-1 (FIGS. 1S-V). Scale bars for=100 μιη in FIGS. 1G-H and 50 μιη in all other panels. **p<0.01 and ***p 0.001 for 2 tailed t-test.

FIGS. 2A-L. BMP2 induces SATB2 and a posterior HOX code in human gut tube spheroids. (FIG. 2A) Schematic of gut tube spheroid patterning protocol. (FIG. 2B-D) BMP signaling levels as measured by pSMAD 1/5/8 (red) staining of spheroids treated with NOGGIN (FIG. 2B), no treatment (FIG. 2C) and BMP2 (FIG. 2D) for 12 hours. (FIG. 2E) pSmad1/5/8 staining of adult mouse colon showing increased BMP signaling at to the top of crypts. (FIGS. 2F-H) SATB2 expression in spheroids treated with NOGGIN (FIG. 2F), no treatment (FIG. 2G) and BMP2 (FIG. 2H) for 72 hours. (FIG. 2I) Quantification of the percentage of SATB2+ CDH1+ epithelium following patterning. (FIG. 2J) Principal component analysis of nascent spheroids and spheroids after 3 days of patterning. (FIG. 2K) Gene ontology analysis of differentially expressed genes between BMP vs NOG treated spheroids. (FIG. 2L) Graph of TPM (Transcripts per million) values of spheroids before and after patterning. Samples analyzed were spheroids before patterning (n=2), and NOGGIN, Control and BMP2 treated spheroids 3 days after patterning (n=4 for each group). For quantification in I, 20 organoids from at least 3 experiments were examined. Error bars represent SD. Scale bars=50 microns. ****p s 0.0001 determined by 2 tailed t-test.

FIGS. 3A-S. Regional patterning is maintained in human intestinal organoids following prolonged in vitro culture. (FIGS. 3A-D) Whole-mount immunofluorescence and QPCR analysis with the proximal marker ONECUT1 (green) of 28-day old organoids that resulted from the initial 3 day treatment of spheroids with NOGGIN, control, or BMP2. Staining with CDX2 (red) and DAPI (blue) were also used to detect the epithelium and mesenchyme. (FIGS. 3E-H) Expression of the posterior marker SATB2 (red) detected by IF and by QPCR. (FIGS. 3I-L) Analysis of the pan-goblet cell marker MUC2 (red) by IF and by QPCR. (FIGS. 3M-P) Analysis of the colon-specific goblet cell marker MUC5B (red) by IF. The number of MUC5B+ cells was quantified in (FIG. 3P). (FIGS. 3Q-S) Analysis of patterning markers in isolated mesenchyme cultures relative to whole organoids. QPCR analysis of CDH1 (FIG. 3Q), the proximal HOX gene HOXD3 (FIG. 3R), and the distal HOX gene HOXA13 (FIG. 3S) in whole organoids and in mesenchyme cultures derived from NOGGIN, control, or BMP2 treated organoids. CDH1 was only observed in whole organoids that contained epithelial cells. Error bars represent SEM. For IF minimum of 10 organoids from at least 3 different experiments were examined for each condition. For QPCR a minimum of 5 biological replicates from 2 separate experiments were examined. Scale bars=100 microns. **p 5 0.01 and ****p 5 0.0001 determined by 2 tailed t-test.

FIGS. 4A-J. HCOs but not HIOs gave rise to colon-specific enteroendocrine cells in response to expression of the proendocrine transcription factor NEUROGENIN 3. (FIGS. 4A-B) Schematic of the doxycycline inducible NEUROG3 lentiviral construct used to generate the IPSC72.3 inducible NEUROG3 line, and the doxycycline induction protocol. Whole-mount staining with Chromagranin A (green), CDX2 (red) and INSL5 (white) of 35 day old organoids patterned with NOGGIN (FIGS. 4C, 4F), untreated (FIGS. 4D,4G) or BMP (FIGS. 4E,4H). (FIGS. 4C-E) Untreated organoids (−Dox) and (FIGS. 4F-H) organoids with expressed NEUROG3 (+Dox). Insets in FIG. 4E and FIG. 4H show a magnified view of INSL5 staining. (FIGS. 4I, 4J) QPCR analysis of NEUROG3 induction of enteroendocrine cells in HIOs and HCOs as measured by CHGA (FIG. 4I) and for INSL5 (FIG. 4J) expression. Data is representative of 2 different experiments with NOGGIN (n=3), Control (n=3) or BMP (n=6) treated organoids. Error bars represent SEM. Scale bars=50 microns. *p<0.05 determined by 2 tailed t-test.

FIGS. 5A-Y. HIOs and HCOs maintained regional identity following transplantation in vivo. (FIGS. 5A-E) H&E staining of biopsies from human jejunum and colon and of NOGGIN-derived HIOs, control HIOs, and BMP2-derived HCOs that were transplanted underneath the mouse kidney capsule and grown for 8-10 weeks in vivo. The samples of the same conditions were stained with the proximal intestinal marker GATA4 (FIGS. 5F-J), the distal intestinal marker SATB2 (FIGS. 5K-O), the Paneth cell marker DEFAS (FIGS. 5P-T), and the colon-specific goblet cell marker MUC5B (FIGS. 5U-Y). Note that although GATA4 and SATB2 double staining was done in different channels but on the same slides for panels (FIGS. 5F-0), they are shown as individual pseudo-colored (red) images. For human biopsies n=2. For transplanted NOGGIN treated organoids n=12, for control organoids n=7, and for BMP2 treated organoids n=16. Scale bars=50 pm.

FIGS. 6A-T. In vivo grown organoids express region-specific hormones. Analysis of expression of the regionally expressed hormones (FIGS. 6A-D) Ghrelin (GHRL), Motilin (MLN), (FIGS. 6E-H) GIP, (FIGS. 6I-L) GLP-1, (FIGS. 6M-P) PYY and (FIGS. 6Q-T) INSL5 in HIOs and HCOs grown for 8-10 weeks underneath the mouse kidney capsule. The proximally enriched hormones GHRL, GIP and MLN were enriched in NOGGIN and control HIOs (FIGS. 6A-H). The distally enriched hormones GLP-1 and PYY were enriched in BMP2-derived HCOs (1-0). The colon specific hormone INSL5 was only present in HCO (FIGS. 6Q-T). Data is representative of a minimum of 5 transplanted organoids per condition. Insets in (FIG. 6A) and (FIG. 6B) show GHRL and MLN double positive cells. (FIGS. 6D, 6H, 6L, 6P, 6T) FPKM values for GHRL, MLN, GIP, GLP1, PYY, and INSL5 are from RNA-seq data. FPKM values represent 3 biological replicates per condition. Scale bars=30 microns.

FIGS. 7A-C. Global transcriptional analysis of HIOs and HCOs and comparison with human small intestine and colon. (FIG. 7A) Principal component analysis human adult and fetal small intestine and colon compared with transplanted HIOs and HCOs. (FIG. 7B) Hypergeometric means test comparing human adult small intestine with HIOs and human adult colon with HCOs. (FIG. 7C) 4-way scatter plot comparing transcripts that were differentially expressed in human small intestine and colon compared to HIOs and HCOs.

FIG. 8A-L. Gata4 and Satb2 mark discreet regional boundaries during development of the small and large intestines. (FIG. 8A) Whole-mount staining of Gata4 (green) and Satb2 (red) in an e9.5 mouse embryo showing expression boundary at the yolk stalk (n=9). (FIG. 8B) Model depicting Gata4 and Satb2 expression domains el 1.5 intestine showing a transitional zone of low Gata4 and low Satb2 expression. (FIG. 8C-E) Whole-mount staining of Gata4 and Satb2 in an el 1.5 mouse embryo showing posterior boundary of Gata4 and anterior boundary of Satb2 at the yolk stalk (n=3). (FIGS. 8F-H) Whole-mount staining of Satb2 and Foxa2 in an e12.5 mouse embryo showing that the anterior boundary of Satb2 expression is maintained (n=3). (FIG. 8I) Whole-mount staining of Gata4 and Satb2 in proximal intestine isolated from an e16.5 mouse embryo (n=6). (FIG. 8J) Whole-mount staining of Gata4 and Satb2 in distal small intestine and large intestine isolated from an e16.5 mouse embryo (n=6). Staining of GATA4 and SATB2 in section of (FIG. 8K) human jejunum (n=2) and (FIG. 8L) colon (n=2). Scale bars=50 μιη (FIGS. 8C-E) and 100 lAm (FIGS. 8F-L). Dotted lines in (FIG. 8C) and (FIG. 8F) mark the approximate location of the umbilicus. Abbreviations: ys, yolk stalk; cb, cecal bud; tz, transition zone; mx, maxilliary; and md, mandibular portion of first brachial arch; ti, terminal ileum; icj, ileocecal junction.

FIGS. 9A-D. SATB2 is expressed in GATA4 negative human small and large intestine. (FIG. 9A) SATB2 staining in human adult duodenum, small intestine, appendix, colon and rectum showing that SATB2 expression is present in distal small intestine and the entire large intestine. (FIG. 9B) Analysis of GATA4 and SATB2 from published RNA-seq data from human adult and fetal intestinal samples. Samples plotted include human adult duodenum (HuSI_Duo_A), human adult small intestine distal to duodenum (HuSI_Dist A), human adult colon (HuColon_A) and human fetal small intestine (HuSI_F). (FIG. 9C) Analysis of GATA4 and SATB2 expression from microarray data generated by Wang et al. 2015 on fetal intestinal stem cells from duodenum (Duo), jejunum (Jej), ileum (lie), ascending colon (AC), transverse colon (TC) and Descending colon grown in Air Liquid Interface (ALI). r2 values were determined using CORREL function in Excel. (FIG. 9D) Principle component analysis, including principle component 1 (PC1), principle component 2 (PC2), and principle component 3 (PC3), on spheroid (spheroid1, spheroid2), NOGGIN (NOG1, NOG2, NOG3, NOG4), control (EGF1, EGF2, EGF3, EGF4), and BMP (BMP1, BMP2, BMP3, BMP4) samples. Samples analyzed were spheroids (n=2), and NOGGIN, Control, and BMP2 treated spheroids (n=4 for each group).

FIGS. 10A-G. BMP mediates SHH activation of posterior HOX genes. (FIG. 10A) Previous model of SHH-mediated activation of posterior HOX genes. (FIG. 10B) New model of SHH mediated activation of posterior HOX genes and BMP-mediated activation of endoderm HOX genes. (FIG. 10C) QPCR analysis of HOX factors following treatment with NOGGIN, control, Smoothened agonist (SAG), or BMP2. (FIG. 10D) Model of BMP4 dependent activation of HOX 13 genes induced by SAG. (FIG. 10E) QPCR analysis of HOXA13 in control, 5 μM SAG, 5 M SAG+NOG and BMP2 treated organoids after 3 days. (FIG. 10F) Model of SHH independent activation of HOX 13 genes induced by exogenous recombinant human BMP2. (FIG. 10G) QPCR analysis of HOXA13 in control, BMP, and BMP+Cyclopamine treated organoids after 3 days (n=6 per condition).

FIG. 11A-L. Extended in vitro culture allows maturation of goblet cells. (FIG. 11A) Quantitation of the percentage of CDX2+ SATB2+ cells in organoids which were patterned and were then re-patterned. QPCR analysis of HOXB13 (FIG. 11B) and HOXD13 (FIG. 11C) in 28-day old organoids. (FIGS. 11D-F) Whole-mount and (FIGS. 11G-1) cross section staining with CDH1 (green), CDX2 (red), and MUC2 (white) from 44-day old NOGGIN, Control, and BMP treated organoids. (FIGS. 11J-L) Staining of sections from 44-day old BMP2 treated organoids. White arrows points to goblet cells which were in the process of secreting Mucin 2. For QPCR a minimum of 5 biological replicates from 2 separate experiments were examined. For IF a minimum of 10 organoids per condition were examined. Scale bars=50 pm.

FIGS. 12A-L. BMP patterning of organoids is stable in vitro and in vivo. (FIG. 12A) Efficiency of organoid engraftment of NOGGIN, Control, and BMP patterned organoids. Quantitation of the percentage of GATA4+ CDX2+ cells (FIG. 12B) and SATB2+ CDX2+ cells (FIG. 12C) in transplanted patterned organoids. FPKM values from RNA-seq data for GATA4 (FIG. 12D), SATB2 (FIG. 12E), DEFAS (FIG. 12F), and MUCSB (FIG. 12G) in transplanted organoids. MUC2 (red) staining of (FIGS. 12H-I) human jejunum and colon biopsies (n=2 per region) and (FIGS. 12J-L) transplanted organoids (n=5 per condition). Scale bars=50 microns.

FIGS. 13A-D′. In vitro and in vivo grown organoids contain intestinal progenitors. Representative whole-mount (FIGS. 13A, 13F, 13K) and slice section (FIGS. 13B, 13G, 13L) images of CDH1 and GFP from H9-LGR5-GFP derived organoids treated with NOGGIN, control, or BMP. CDX2 (red) and SOX9 (green) staining on sections from (FIGS. 13C-E) NOGGIN, (FIGS. 13H-J) control, or (FIGS. 13M-0) BMP2 treated organoids. Representative images of CDX2 and LGR5-GFP (FIGS. 13P, 13S, 13V), CDX2 and SOX9 (FIGS. 13Q, 13T, 13W), and CDH1 and KI67 (FIGS. 13R, 13U, 13X) stained in vivo organoids derived from H9-LGR5-GFP organoids treated with NOGGIN, control, or BMP. (FIGS. 13Y-A′) Stereomicrographs showing enteroids derived from NOGGIN, control or BMP transplants respectively. (FIGS. 13B‘-D’) QPCR analysis of proximal and distal genes in control enteroids (>100 pooled enteroids from 2 transplants) and BMP2 treated colonoids (>50 colonoids from 1 transplant). Scale bars=50 μιη.

FIGS. 14A-C. Ribosome and immune cell signatures are differentially expressed between transplanted organoids and primary human tissues. (FIG. 14A) Principal component analysis of patterned transplanted organoids and human adult and fetal small intestine and colon. (FIG. 14B) Gene ontology analysis of genes upregulated in transplants versus human primary tissues. (FIG. 14C) Gene ontology analysis of genes upregulated in human primary tissues versus transplants.

FIGS. 15A-D. (FIG. 15A) Wholemount immunofluorescence staining of HCOs after 15 days of growth in Matrigel. HCO cultures were stained for the endothelial marker CD31 (green) and the hindgut epithelium marker CDX2 (red). Cultures were also stained for the hematopoietic cell marker PU.1 (red right panel). (FIG. 15B) Schematic of hematopoietic progenitor assays. Cells were collected from HCOs, centrifuged and either stained using Giemsa Wright Stain or plated in Methocult media to assay for hematopoietic cell differentiation. (FIG. 15C) Representative images of Giemsa Wright stained cells with morphologies consistent with differentiation into Macrophages, Neutrophils, Basophils and Eosinophils. (FIG. 15D) Representative images of colonies formed after 14 days in Methocult. Erythrocyte, macrophage and granulocyte colonies were present in cells derived from HCOs but not those derived from NOGGIN treated HIOs.

FIGS. 16A-D. (FIG. 16A) Immunofluorescence staining of a human colon biopsy or an HCO grown for 28 days in Matrigel. Staining was done for CD68 a marker of macrophages. (FIG. 16B) Plots of CYTOF analysis of CD14 and CD16 in HIOs and HCOs. A small percentage of CD14+/CD16+ cells are present in HCOs (blue square) but not HIOs. Additionally, CD 16 single positive cells were present in HCOs suggesting monocytes are present within the culture. (FIG. 16C) Luminex array analysis of supernatant collected from 14 and 28-day old HIOs and HCOs. IL6 and IL8 were detected in 28-day old HCOs (BMP) but not HIOs. (FIG. 16D) Luminex array analysis of supernatant collected from 14 and 28-day old HIOs and HCOs. The macrophage specific cytokines MIP1A and MIP1B were detected in 14 and 28-day old HCOs (BMP) but not in 14 or 28-day old HIOs.

### DETAILED DESCRIPTION

### Definitions

Unless otherwise noted, terms are to be understood according to conventional usage by those of ordinary skill in the relevant art.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “totipotent stem cells” (also known as omnipotent stem cells) are stem cells that can differentiate into embryonic and extraembryonic cell types. Such cells can construct a complete, viable, organism. These cells are produced from the fusion of an egg and sperm cell. Cells produced by the first few divisions of the fertilized egg are also totipotent.

As used herein, the term “pluripotent stem cells (PSCs),” also commonly known as PS cells, encompasses any cells that can differentiate into nearly all cells, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of totipotent cells, derived from embryonic stem cells (including embryonic germ cells) or obtained through induction of a non-pluripotent cell, such as an adult somatic cell, by forcing the expression of certain genes.

As used herein, the term “induced pluripotent stem cells (iPSCs),” also commonly abbreviated as iPS cells, refers to a type of pluripotent stem cells artificially derived from a normally non-pluripotent cell, such as an adult somatic cell, by inducing a “forced” expression of certain genes.

As used herein, the term “embryonic stem cells (ESCs),” also commonly abbreviated as ES cells, refers to cells that are pluripotent and derived from the inner cell mass of the blastocyst, an early-stage embryo. For purpose of the present invention, the term “ESCs” is used broadly sometimes to encompass the embryonic germ cells as well.

As used herein, the term “precursor cell” encompasses any cells that can be used in methods described herein, through which one or more precursor cells acquire the ability to renew itself or differentiate into one or more specialized cell types. In some aspects, a precursor cell is pluripotent or has the capacity to becoming pluripotent. In some aspects, the precursor cells are subjected to the treatment of external factors (e.g., growth factors) to acquire pluripotency. In some aspects, a precursor cell can be a totipotent (or omnipotent) stem cell; a pluripotent stem cell (induced or non-induced); a multipotent stem cell; an oligopotent stem cells and a unipotent stem cell. In some aspects, a precursor cell can be from an embryo, an infant, a child, or an adult. In some aspects, a precursor cell can be a somatic cell subject to treatment such that pluripotency is conferred via genetic manipulation or protein/peptide treatment.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

As used herein, the term “cellular constituents” are individual genes, proteins, mRNA expressing genes, and/or any other variable cellular component or protein activities such as the degree of protein modification (e.g., phosphorylation), for example, that is typically measured in biological experiments (e.g., by microarray or immunohistochemistry) by those skilled in the art. Significant discoveries relating to the complex networks of biochemical processes underlying living systems, common human diseases, and gene discovery and structure determination can now be attributed to the application of cellular constituent abundance data as part of the research process. Cellular constituent abundance data can help to identify biomarkers, discriminate disease subtypes and identify mechanisms of toxicity.

As described herein, methods and systems are established using a temporal series of growth factor manipulations to mimic embryonic intestinal development in culture. In particular, methods and systems are established to direct in vitro differentiation of PSCs, both human embryonic stem cells (hESC) and induced pluripotent stem cells (iPSC), into intestinal tissue

The generation of gastric and small intestinal organoids from pluripotent stem cells (PSCs) has revolutionized the study human gastrointestinal (GI) development and disease. However, efforts to generate large intestinal organoids have lagged behind, in part due to a robust molecular understanding of posterior gut tube development. Here, Applicant has found that the intestinal epithelium posterior to the umbilical cord expresses Satb2 throughout development and postnatally. Applicant has further found that BMP signaling establishes the Satb2+ domain in frog and mouse embryos, and that brief activation of BMP signaling was sufficient to activate a posterior HOX code and direct human PSC-derived gut tube cultures into colonic organoids (HCOs). HCOs grown in vitro had a marker profile and unique cell types consistent with colonic identity. Following transplantation into mice, HCOs underwent morphogenesis and maturation forming tissue with molecular, cellular and morphologic properties of the human colon. The disclosed colonic organoids may be used in future studies of colitis and colon cancer.

In one aspect, a method of inducing formation of a human colon organoid is disclosed. The method may comprise the steps of (a) contacting a definitive endoderm (DE) with an FGF signaling pathway activator and a WNT signaling pathway activator (for example, CHIRON/GSK2 inhibitor) for a period of time sufficient for said DE to form a mid-hindgut spheroid, and (b) contacting the mid-hindgut spheroid of step (a) with a BMP activator and an EGF signaling pathway activator for a period of time sufficient to form said human colon organoid, wherein said human colon organoid expresses SATB2.

In one aspect, the DE may be derived from a precursor cell selected from an embryonic stem cell, an embryonic germ cell, an induced pluripotent stem cell, a mesoderm cell, a definitive endoderm cell, a posterior endoderm cell, a hindgut cell or combinations thereof.

In one aspect, the FGF signaling pathway activator may be selected from a small molecule or protein FGF signaling pathway activator, FGF1, FGF2, FGF3, FGF4, FGF10, FGF11, FGF12, FGF13, FGF14, FGF15, FGF16, FGF17, FGF18, FGF19, FGF20, FGF21, FGF22, FGF23, or combinations thereof. The WNT signaling pathway activator may be selected from a small molecule or protein Wnt signaling pathway activator, preferably Lithium Chloride; 2-amino-4,6-disubstituted pyrimidine (hetero) arylpyrimidines; IQ1; QS11; NSC668036; DCA beta-catenin; 2-amino-4-[3,4-(methylenedioxy)-benzyl-amino]-6-(3-methoxyphenyl) pyrimidine, Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt1Oa, Wnt1Ob, Wnt1 1, Wnt1 6, a GSK3 inhibitor, preferably CHIRON, or combinations thereof. In one aspect, the BMP activator may be selected from BMP2, BMP4, BMP7, BMP9, small molecules that activates the BMP pathway, proteins that activate the BMP pathway, and may include the following: Noggin, Dorsomorphin, LDN189, DMH-1, ventromophins, and combinations thereof.

In one aspect, the period of time sufficient for said DE to form a mid-hindgut spheroid may be determined by expression of CDX2 by said mid-hindgut spheroid of step (a). Such measurement is within the ability of one of ordinary skill in the art using routine methods.

In one aspect, the period of time sufficient for the mid-hindgut spheroid to form a human colon organoid is determined by expression of SATB2 and CDX2 by a cell of said human colon organoid, wherein when SATB2 and CDX2 is expressed, the mid-hindgut spheroid has formed a human colon organoid. Such measurement may be used in lieu of a temporal measurement, in that expression of the genes listed above indicates that steps (a) and (b) have been carried out for a sufficient duration of time.

In one aspect, an HCO obtained according to the methods described herein are disclosed. The HCOs of the instant invention may be characterized in a variety of different ways. In one aspect, the HCO may be characterized by the presence of colonic enteroendocrine cells (EEC). In one aspect, the HCO may be characterized by the presence of crypts and is substantially free of villi. In one aspect, the HCO may be characterized by the presence of colon-specific goblet cells. In one aspect, the HCO may be characterized by being substantially free of Paneth cells. In one aspect, the HCO may be characterized by the ability to secrete colon-specific hormone INSL5. The intestinal organoid may be free of one or more of an immune function, innervation, blood vessels, villi, and Paneth cells.

In one aspect, a method of forming colonic tissue is disclosed, wherein the HCO of the described invention may be engrafted under a kidney capsule of a mammal, preferably a rodent, preferably an immunocompromised rodent, preferably an immunocompromised mouse.

In one aspect, the HCOs disclosed herein may be used to determine the efficacy and/or toxicity of a potential therapeutic agent for a disease selected from colitis, colon cancer, polyposis syndromes, and/or irritable bowel syndrome. The method may comprise the step of contacting a potential therapeutic agent with an HCO as described herein, for a period of time sufficient to determine the efficacy and/or toxicity of said potential therapeutic agent.

In one aspect, an intestinal colonoid derived from the HCO of any preceding claim is contemplated.

In some aspects, stem cells that are pluripotent or can be induced to become pluripotent may be used. In some aspects, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. For example, three cell lines (HI, H13, and H14) had a normal XY karyotype, and two cell lines (H7 and H9) had a normal XX karyotype. Human embryonic stem cells H9 (H9-hESCs) are used in the exemplary aspects described in the present application, but it would be understood by one of skill in the art that the methods and systems described herein are applicable to any stem cells.

Additional stem cells that can be used in aspects in accordance with the present invention include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, California); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in aspects in accordance with the present invention include but are not limited to SA01 (SA001); SA02 (SA002); ESO1 (HES-1); ES02 (HES-2); ES03 (HES-3); ES04 (HES-4); ES05 (HES-5); ES06 (HES-6); BGO1 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14).

In some aspects, the stem cells are further modified to incorporate additional properties. Exemplary modified cell lines include but not limited to HI OCT4-EGFP; H9 Cre-LoxP; H9 hNanog-pGZ; H9 hOct4-pGZ; H9 inGFPhES; and H9 Syn-GFP.

More details on embryonic stem cells can be found in, for example, Thomson et al., 1998, “Embryonic Stem Cell Lines Derived from Human Blastocysts,” Science 282 (5391): 1145-1147; Andrews et al, 2005, “Embryonic stem (ES) cells and embryonal carcinoma (EC) cells: opposite sides of the same coin,” Biochem Soc Trans 33: 1526-1530; Martin 1980, “Teratocarcinomas and mammalian embryogenesis,”. Science 209 (4458):768-776; Evans and Kaufman, 1981, “Establishment in culture of pluripotent cells from mouse embryos,” Nature 292(5819): 154-156; Klimanskaya et al., 2005, “Human embryonic stem cells derived without feeder cells,” Lancet 365 (9471): 1636-1641; each of which is hereby incorporated herein in its entirety.

Alternatively, pluripotent stem cells can be derived from embryonic germ cells (EGCs), which are the cells that give rise to the gametes of organisms that reproduce sexually. EGCs are derived from primordial germ cells found in the gonadal ridge of a late embryo, have many of the properties of embryonic stem cells. The primordial germ cells in an embryo develop into stem cells that in an adult generate the reproductive gametes (sperm or eggs). In mice and humans, it is possible to grow embryonic germ cells in tissue culture under appropriate conditions. Both EGCs and ESCs are pluripotent. For purpose of the present invention, the term “ESCs” is used broadly sometimes to encompass EGCs.

Induced Pluripotent Stem Cells (iPSCs)

In some aspects, iPSCs are derived by transfection of certain stem cell-associated genes into non-pluripotent cells, such as adult fibroblasts. Transfection may be achieved through viral vectors, such as retroviruses. Transfected genes include the master transcriptional regulators Oct-3/4 (Pouf51) and Sox2, although it is suggested that other genes enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include but are not limited to first generation iPSCs, second generation iPSCs in mice, and human induced pluripotent stem cells.

In some aspects, non-viral based technologies may be employed to generate iPSCs. In some aspects, an adenovirus can be used to transport the requisite four genes into the DNA of skin and liver cells of mice, resulting in cells identical to embryonic stem cells. Since the adenovirus does not combine any of its own genes with the targeted host, the danger of creating tumors is eliminated. In some aspects, reprogramming can be accomplished via plasmid without any virus transfection system at all, although at very low efficiencies. In other aspects, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some aspects, the expression of pluripotency induction genes can also be increased by treating somatic cells with FGF2 under low oxygen conditions.

More details on embryonic stem cells can be found in, for example, Kaji et al, 2009, “Virus free induction of pluripotency and subsequent excision of reprogramming factors,” Nature 458:771-775; Woltjen et al, 2009, “piggyBac transposition reprograms fibroblasts to induced pluripotent stem cells,” Nature 458:766-770; Okita et al., 2008, “Generation of Mouse Induced Pluripotent Stem Cells Without Viral Vectors,” Science 322(5903):949-953; Stadtfeld et al., 2008, “Induced Pluripotent Stem Cells Generated without Viral Integration,” Science 322(5903):945-949; and Zhou et al., 2009, “Generation of Induced Pluripotent Stem Cells Using Recombinant Proteins,” Cell Stem Cell 4(5):381-384; each of which is hereby incorporated herein in its entirety.

In some aspects, exemplary iPS cell lines include but not limited to iPS-DF19-9; iPS-DF19-9; iPS-DF4-3; iPS-DF6-9; iPS (Foreskin); iPS(IMR90); and iPS(IMR90).

### Definitive Endoderm

The HCOs of the instant disclosure may be derived from a simple sheet of cells called the definitive endoderm (DE). Methods for deriving definitive endoderm from precursor cells are well known in the art, as taught by D' Armour et al. 2005 and Spence et al. The anterior DE forms the foregut and its associated organs including the liver and pancreas and the posterior DE forms the midgut and hindgut, which forms the small and large intestines and parts of the genitourinary system. Studies using mouse, chick and frog embryos suggest that establishing the anterior-posterior pattern in DE at the gastrula stage is a prerequisite for subsequent foregut and hindgut development. The Wnt and FGF signaling pathways are believed to be critical for this process and act to promote posterior endoderm and hindgut fate and suppress anterior endoderm and foregut fate. The simple cuboidal epithelium of the hindgut first develops into a pseudostratified columnar epithelium, then into villi containing a polarized columnar epithelium and a proliferative zone at the base of the villi, which corresponds with the presumptive progenitor domain.

Applicant describes herein a robust and efficient process to direct the differentiation of DE into intestinal tissue, in particular human colon tissue, in vitro. Directed differentiation may be achieved by selectively activating certain signaling pathways in the iPSCs and/or DE cells.

Additional details of pathways relating to intestinal development in general are found in, for example, Sancho et al., 2004, “Signaling Pathways in Intestinal Development and Cancer,” Annual Review of Cell and Developmental Biology 20:695-723; Logan and Nusse, 2004, “The Wnt Signaling Pathway in Development and Disease,” Annual Review of Cell and Developmental Biology 20:781-810; Taipalel and Beachyl, 2001, “The Hedgehog and Wnt signalling pathways in cancer,” Nature 411:349-354; Gregorieff and Clevers, 2005, “Wnt signaling in the intestinal epithelium: from endoderm to cancer,” Genes & Dev. 19: 877-890; each of which is hereby incorporated by reference herein in its entirety. More details on the functions of signaling pathways relating to DE development can be found in, for example, Zorn and Wells, 2009, “Vertebrate endoderm development and organ formation,” Annu Rev Cell Dev Biol 25:221-251; Dessimoz et al., 2006, “FGF signaling is necessary for establishing gut tube domains along the anterior-posterior axis in vivo,” Mech Dev 123:42-55; McLin et al., 2007, “Repression of Wnt/{beta}-catenin signaling in the anterior endoderm is essential for liver and pancreas development. Development,” 134:2207-2217; Wells and Melton, 2000, Development 127: 1563-1572; de Santa Barbara et al, 2003, “Development and differentiation of the intestinal epithelium,” Cell Mol Life Sci 60(7): 1322-1332; each of which is hereby incorporated herein in its entirety.

Any methods for producing definitive endoderm from pluripotent cells (e.g., iPSCs or ESCs) are applicable to the methods described herein. In some aspects, pluripotent cells are derived from a morula. In some aspects, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some aspects, human embryonic stem cells are used to produce definitive endoderm. In some aspects, human embryonic germ cells are used to produce definitive endoderm. In some aspects, iPSCs are used to produce definitive endoderm.

In some aspects, one or more growth factors are used in the differentiation process from pluripotent stem cells to DE cells. The one or more growth factors used in the differentiation process can include growth factors from the TGF-beta superfamily. In such aspects, the one or more growth factors may comprise the Nodal/Activin and/or the BMP subgroups of the TGF-beta superfamily of growth factors. In some aspects, the one or more growth factors are selected from the group consisting of Nodal, Activin A, Activin B, BMP4, Wnt3a or combinations of any of these growth factors. In some aspects, the embryonic stem cells or germ cells and iPSCs are treated with the one or more growth factors for 6 or more hours; 12 or more hours; 18 or more hours; 24 or more hours; 36 or more hours; 48 or more hours; 60 or more hours; 72 or more hours; 84 or more hours; 96 or more hours; 120 or more hours; 150 or more hours; 180 or more hours; or 240 or more hours. In some aspects, the embryonic stem cells or germ cells and iPSCs are treated with the one or more growth factors at a concentration of 10 ng/ml or higher; 20 ng/ml or higher; 50 ng/ml or higher; 75 ng/ml or higher; 100 ng/ml or higher; 120 ng/ml or higher; 150 ng/ml or higher; 200 ng/ml or higher; 500 ng/ml or higher; 1,000 ng/ml or higher; 1,200 ng/ml or higher; 1,500 ng/ml or higher; 2,000 ng/ml or higher; 5,000 ng/ml or higher; 7,000 ng/ml or higher; 10,000 ng/ml or higher; or 15,000 ng/ml or higher. In some aspects, concentration of the growth factor is maintained at a constant level throughout the treatment. In other aspects, concentration of the growth factor is varied during the course of the treatment. In some aspects, the growth factor is suspended in media that include fetal bovine serine (FBS) with varying HyClone concentrations. One of skill in the art would understand that the regimen described herein is applicable to any known growth factors, alone or in combination. When two or more growth factors are used, the concentration of each growth factor may be varied independently.

In some aspects, populations of cells enriched in definitive endoderm cells are used. In some aspects, the definitive endoderm cells are isolated or substantially purified. In some aspects, the isolated or substantially purified definitive endoderm cells express the SOX17, FOXA2, and/or the CXRC4 marker to a greater extent than the OCT4, AFP, TM, SPARC and/or SOX7 markers. Methods for enriching a cell population with definitive endoderm are also contemplated. In some aspects, definitive endoderm cells can be isolated or substantially purified from a mixed cell population by contacting the cells with a reagent that binds to a molecule that is present on the surface of definitive endoderm cells but which is not present on the surface of other cells in the mixed cell population, and then isolating the cells bound to the reagent. In certain aspects, the cellular constituent that is present on the surface of definitive endoderm cells is CXCR4.

Additional methods for obtaining or creating DE cells that can be used in the present invention include but are not limited to those described in U.S. Pat. No. 7,510,876 to D'Amour et al; U.S. Pat. No. 7,326,572 to Fisk et al.; Kubol et al., 2004, “Development of definitive endoderm from embryonic stem cells in culture,” Development 131:1651-1662; D'Amour et al, 2005, “Efficient differentiation of human embryonic stem cells to definitive endoderm,” Nature Biotechnology 23:1534-1541; and Ang et al, 1993, “The formation and maintenance of the definitive endoderm lineage in the mouse: involvement of HNF3/forkhead proteins,” Development 119: 1301-1315; each of which is hereby incorporated by reference herein in its entirety.

### Definitive Endoderm to Mid/Hindgut Spheroids

In some aspects, posteriorized endoderm cells of the DE are further developed into one or more specialized cell types. Activin-induced definitive endoderm (DE) can further undergo FGF/Wnt induced posterior endoderm pattering, hindgut specification and morphogenesis, and finally a pro-intestinal culture system that promoted intestinal growth, morphogenesis and cytodifferentiation into functional intestinal cell types including enterocytes, goblet, Paneth and enteroendocrine cells. In some aspects, human PSCs are efficiently directed to differentiate in vitro into intestinal epithelium that may include secretory, endocrine and absorptive cell types. It will be understood that molecules such as growth factors may be added to any stage of the development to promote a particular type of intestinal tissue formation.

PSCs, such as ESCs and iPSCs, undergo directed differentiation in a step-wise or non-step-wise manner first into definitive endoderm (DE) then into mid/hindgut epithelium and mesenchyme (e.g., hindgut spheroids), and then into intestinal tissue. In some aspects, definitive endoderm cells and hESCs are treated with one or more growth factors.

In some aspects, soluble FGF and Wnt ligands are used to mimic early hindgut specification in culture to convert, through directed differentiation, DE developed from iPSCs or ESCs into hindgut epithelium that efficiently gives rise to all the major intestinal cell types. In human, directed differentiation of DE is achieved through selective activating certain signaling pathways that are important to intestinal development. It will be understood by one of skill in the art that altering the expression of any Wnt signaling protein in combination with any FGF ligand can give rise to directed differentiation as described herein.

More details are found, for example, in Liu et al., “A small-molecule agonist of the Wnt signaling pathway,” Angew Chem Int Ed Engl. 44(13): 1987-1990 (2005); Miyabayashi et al, “Wnt/beta-catenin/CBP signaling maintains long-term murine embryonic stem cell pluripotency,” Proc Natl Acad Sci USA. 104(13):5668-5673 (2007); Zhang et al; “Small-molecule synergist of the Wnt/beta-catenin signaling pathway,” Proc Natl Acad Sci U SA. 104(18):7444-7448 (2007); Neiiendam ei /., “An NCAM-derived FGF-receptor agonist, the FGL-peptide, induces neurite outgrowth and neuronal survival in primary rat neurons,” J Neurochem. 91(4):920-935 (2004); Shan et al, “Identification of a specific inhibitor of the dishevelled PDZ domain,” Biochemistry 44(47): 15495-15503 (2005); Coghlan et al, “Selective small molecule inhibitors of glycogen synthase kinase-3 modulate glycogen metabolism and gene transcription,” Chem Biol. 7(10):793-803 (2000); Coghlan et al, “Selective small molecule inhibitors of glycogen synthase kinase-3 modulate glycogen metabolism and gene transcription,” Chemistry & Biology 7(10):793-803; and Pai et al, “Deoxycholic acid activates beta-catenin signaling pathway and increases colon cell cancer growth and invasiveness,” Mol Biol Cell. 15(5):2156-2163 (2004); each of which is hereby incorporated by reference in its entirety.

In some aspects, siRNA and/or shRNA targeting cellular constituents associated with the Wnt and/or FGF signaling pathways are used to activate these pathways.

Modulators/activators of the Wnt signaling pathway include Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt1Oa, Wnt1Ob, Wnt1 1, and Wnt16. In some aspects, the modulation of the pathway may be through the use of small molecule modulators or protein modulators that activate the aforementioned pathways or proteins that activate the aforementioned pathways. For example, Small molecule modulators of the Wnt pathway included, but is not limited to Lithium Chloride; 2-amino-4,6-disubstituted pyrimidine (hetero) arylpyrimidines; IQ1; QS11; NSC668036; DCA beta-catenin; 2-amino-4-[3,4-(methylenedioxy)-benzyl-amino]-6-(3-methoxyphenyl) pyrimidine. Exemplary natural inhibitors of Wnt signaling include but are not limited to Dkkl, SFRP proteins and FrzB. In some aspects, the extrinsic molecules include but are not limited to small molecules such as WAY-316606; SB-216763; or BIO (6-bromoindirubin-3′-oxime). In some aspects, siRNA and/or shRNA targeting cellular constituents associated with the Wnt and/or FGF signaling pathways may be used to activate these pathways. It would be understood by one of skill in the art that the target cellular constituents include but are not limited to SFRP proteins; GSK3, Dkkl, and FrzB. Additional modulators include molecules or proteins that inhibit GSK3, which activates the Wnt signaling pathway. Exemplary GSK3 inhibitors include, but are not limited to: Chiron/CHIR99021, for example, which inhibits GSK3. One of ordinary skill in the art will recognize GSK3 inhibitors suitable for carrying out the disclosed methods. The GSK3 inhibitor may be administered in an amount of from about 1 uM to about 100 uM, or from about 2 uM to about 50 uM, or from about 3 uM to about 25 uM. One of ordinary skill in the art will readily appreciate the appropriate amount and duration.

Fibroblast growth factors (FGFs) are a family of growth factors involved in angiogenesis, wound healing, and embryonic development. In some aspects, it will be understood by one of skill in the art that any of the FGFs can be used in conjunction with a protein from the Wnt signaling pathway. In some aspects, soluble FGFs include and but are not limited to FGF4, FGF2, and FGF3. In some embodiments, the FGF signaling pathway is activated by contacting the precursor cell with one or more molecules selected from the group consisting of FGF1, FGF2, FGF3, FGF4, FGF10, FGF11, FGF12, FGF13, FGF14, FGF15, FGF16, FGF17, FGF18, FGF19, FGF20, FGF21, FGF22, and FGF23. In some embodiments, siRNA and/or shRNA targeting cellular constituents associated with the FGF signaling pathway may be used to activate these pathways. It will be understood by one of skill in the art that the methods and compositions described herein in connection with the Wnt and FGF signaling pathways are provided by way of examples. Similar methods and compositions are applicable to other signaling pathways disclosed herein.

In some aspects, DE culture is treated with the one or more modulators of a signaling pathway described herein for 6 or more hours; 12 or more hours; 18 or more hours; 24 or more hours; 36 or more hours; 48 or more hours; 60 or more hours; 72 or more hours; 84 or more hours; 96 or more hours; 120 or more hours; 150 or more hours; 180 or more hours; 200 or more hours, 240 or more hours; 270 or more hours; 300 or more hours; 350 or more hours; 400 or more hours; 500 or more hours; 600 or more hours; 700 or more hours; 800 or more hours; 900 or more hours; 1,000 or more hours; 1,200 or more hours; or 1,500 or more hours.

In some aspects, DE culture is treated with the one or more modulators of a signaling pathway described herein at a concentration of 10 ng/ml or higher; 20 ng/ml or higher; 50 ng/ml or higher; 75 ng/ml or higher; 100 ng/ml or higher; 120 ng/ml or higher; 150 ng/ml or higher; 200 ng/ml or higher; 500 ng/ml or higher; 1,000 ng/ml or higher; 1,200 ng/ml or higher; 1,500 ng/ml or higher; 2,000 ng/ml or higher; 5,000 ng/ml or higher; 7,000 ng/ml or higher; 10,000 ng/ml or higher; or 15,000 ng/ml or higher. In some aspects, concentration of signaling molecule is maintained at a constant throughout the treatment. In other aspects, concentration of the modulators of a signaling pathway is varied during the course of the treatment. In some aspects, a signaling molecule in accordance with the present invention is suspended in media comprising DMEM and fetal bovine serine (FBS). The FBS can be at a concentration of 2% and more; 5% and more; 10% or more; 15% or more; 20% or more; 30% or more; or 50% or more. One of skill in the art would understand that the regiment described herein is applicable to any known modulators of the signaling pathways described herein, alone or in combination, including but not limited to any molecules in the Wnt and FGF signaling pathways.

In aspects where two or more signaling molecules are used to treat the DE culture, the signaling molecules can be added simultaneously or separately. When two or more molecules are use, the concentration of each may be varied independently.

Expression of CDX2 may be used to reveal tendency of hindgut formation after DE have been incubated with an FGF signaling activator and a Wnt signaling activator, for example, FGF4 and Wnt3a, for a period of time, for example, for 12 hours or longer; 18 hours or longer; 24 hours or longer; 36 hours or longer; 48 hours or longer; 60 hours or longer; or 90 hours or longer. In some aspects, longer periods of incubation are needed to achieve a stable posterior endoderm phenotype as measured by prolonged expressed of CDX2. In such aspects, the periods of incubation can be for 60 hours or longer; 72 hours or longer; 84 hours or longer; 96 hours or longer; 108 hours or longer; 120 hours or longer; 140 hours or longer; 160 hours or longer; 180 hours or longer; 200 hours or longer; 240 hours or longer; or 300 hours or longer.

Alternatively, in some aspects, the absence of cellular constituents, such as foregut markers Sox2, Pdx1, Cldn18, and Albumin, can be used to reveal directed hindgut formation. In some aspects, intestinal transcription factors CDX2, KLF5 and SOX9 can be used to represent intestinal development. In some aspects, GATA6 protein expression can be used to represent intestinal development. In these aspects, the periods of incubation can be for 12 hours or longer; 18 hours or longer; 24 hours or longer; 36 hours or longer; 48 hours or longer; 60 hours or longer; or 90 hours or longer. Alternatively, the periods of incubation can be for 60 hours or longer; 72 hours or longer; 84 hours or longer; 96 hours or longer; 108 hours or longer; 120 hours or longer; 140 hours or longer; 160 hours or longer; 180 hours or longer; 200 hours or longer; 240 hours or longer; or 300 hours or longer.

In some aspects, abundance data of cellular constituents, for example, protein and/or gene expression levels, are determined by immunohistochemistry using primary and/or secondary antibodies targeting molecules in the relevant signaling pathways. In other aspects, abundance data of cellular constituents, for example, protein and/or gene expression levels, are determined by microarray analyses.

Still alternatively, morphological changes can be used to represent the progress of directed differentiation. In some aspects, hindgut spheroids are further subject to 3-dimensional culture conditions for further maturation. In other aspects, a highly convoluted epithelium surrounded by mesenchymal cells can be observed following hindgut spheroids formation. Additionally, intestinal organoids; polarized columnar epithelium; goblet cells; or smooth muscle cells can be observed in 6 days or longer; 7 days or longer; 9 days or longer; 10 days or longer; 12 days or longer; 15 days or longer; 20 days or longer; 25 days or longer; 28 days or longer; 32 days or longer; 36 days or longer; 40 days or longer; 45 days or longer; 50 days or longer; or 60 days or longer.

### Mid/Hindgut Spheroids to Colon Organoids

It has been identified that, in addition to FGF and WNT signaling, Bone Morphogenetic Proteins (BMP) specifically BMP2 and BMP4, are capable of promoting a posterior/hindgut fate and repressing foregut fate. Additionally, BMP signaling regulates formation of distinct regional types of intestine. Inhibition of BMP with noggin after the hindgut stage promotes a proximal intestinal fate (duodenum/jejunum). Activation of BMP signaling after the hindgut stage promotes a more distal intestinal cell fate (cecum/colon).

Activation of BMP can be carried out by contacting the mid/hindgut spheroids with a BMP activator and an EGF signaling pathway activator for a period of time sufficient to form said human colon organoid. The demarcation of the incubation period may be defined by the point in time in which the human colon organoid expresses SATB2. Suitable BMP activators and EGF signaling pathway activators will be readily appreciated by one of ordinary skill in the art. Suitable BMP activators may include, for example BMP2, BMP4, BMP7, BMP9 and protein or small molecule agonists such as ventromorphins (Genthe et al. 2017) or proteins that serve as agonists. The BMP activator and EGF signaling pathway activator may be contacted with the mid-/hindgut spheroids for from about 1 day to about 3 days. BMP signaling may be activated within the first three days. In one aspect, the contacting step of the BMP activator and EGF signaling pathway activator is from 24 hours to about 10 days, or from about 48 hours to about 9 days, or from about 3 days to about 8 days, or from about 4 days to about 8 days, or from about 5 days to about 7 days. Suitable EGF activators may include, for example TGF alpha, HB-EGF, Amphiregulin, Epigen, Betacellulin and small molecules such as db-cAMP. The EGF activator may be contacted with the mid-/hindgut spheroids at a concentration of from about 10 ng/mL to 10,000 ng/MVL, for a time period of from about 24 hours to about 10 days, or from about 48 hours to about 9 days, or from about 3 days to about 8 days, or from about 4 days to about 8 days, or from about 5 days to about 7 days.

The mid/hindgut spheroids may be contacted with a BMP activator and/or EGF activator at a concentration of 5 ng/ml or higher; 20 ng/ml or higher; 50 ng/ml or higher; 75 ng/ml or higher; 100 ng/ml or higher; 120 ng/ml or higher; 150 ng/ml or higher; 200 ng/ml or higher; 500 ng/ml or higher; 1,000 ng/ml or higher; 1,200 ng/ml or higher; 1,500 ng/ml or higher; 2,000 ng/ml or higher; 5,000 ng/ml or higher; 7,000 ng/ml or higher; 10,000 ng/ml or higher; or 15,000 ng/ml or higher, alone or combined. In some embodiments, concentration of signaling molecule is maintained at a constant throughout the treatment. In other embodiments, concentration of the molecules of a signaling pathway is varied during the course of the treatment. In some embodiments, a signaling molecule in accordance with the present invention is suspended in media comprising DMEM and fetal bovine serine (FBS). The FBS can be at a concentration of 2% and more; 5% and more; 10% or more; 15% or more; 20% or more; 30% or more; or 50% or more. One of skill in the art would understand that the regiment described herein is applicable to any known molecules of the signaling pathways described herein, alone or in combination

### EXAMPLES

The following non-limiting examples are provided to further illustrate aspects of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific aspects that are disclosed and still obtain a like or similar result without departing from the spirit and scope of the invention.

The epithelium of the gastrointestinal tract is derived from the definitive endoderm, one of the primary germ layers that are established during gastrulation. The process of gut tube morphogenesis transforms the definitive endoderm into a primitive gut tube with a foregut, midgut and hindgut. The midgut gives rise to the small and proximal large intestine and the hindgut gives rise to the distal large intestine and rectum (Zorn and Wells, 2009). The small intestine is further subdivided into 3 segments: The duodenum which is involved in absorption of nutrients and uptake of iron, the jejunum which is involved in the digestion and absorption of nutrients and the ileum which is involved in the absorption of bile acids and vitamin-B12 (Jeejeebhoy, 2002). The large intestine is subdivided in to the cecum, colon and rectum which are all involved in absorption of water and electrolytes (Jeejeebhoy, 2002). Although recent advances have shed light into the development of the small intestine (Finkbeiner et al., 2015; Spence et al., 2011; Watson et al., 2014), little is known about development of human large intestine/colon. Furthermore, diseases affecting this region of the gastrointestinal (GI) tract, colitis, colon cancer, polyposis syndromes and Irritable Bowel Syndrome are prevalent (Molodecky et al., 2012; Siegel et al., 2014; Zbuk and Eng, 2007). Animal models of polyposis syndromes and intestinal cancer are limited since polyps and tumors preferentially form in the small intestine and rarely in the colon or rectum (Haramis et al., 2004; He et al., 2004; Moser et al., 1990).

Applicant previously described a method in which human pluripotent stem cells can be differentiated into intestinal tissue through steps of directed differentiation that approximate embryonic development of the small intestine. First, pluripotent stem cells are differentiated into definitive endoderm by treatment with Activin A. Exposure of definitive endoderm to high levels of Wnt and FGF induces morphogenesis into mid/hindgut tube spheroids. Once formed, these midgut/hindgut spheroids, when grown in 3-dimensional culture under conditions that favor intestinal growth, transition through stages that approximate small intestinal development in vivo and form human intestinal organoids (HIOs) (Spence et al., 2011). HIOs have a small intestinal identity and have proven extremely useful for modeling small intestinal biology (Bouchi et al., 2014; Finkbeiner et al., 2015; Watson et al., 2014; Xue et al., 2013). However, until now, PSC-derived large intestinal organoids have not been developed, and given the prevalence of disease in the large intestine, such a system would allow for interrogation of development and disease mechanisms in this region of the GI tract.

To develop a method for generating large intestinal organoids, Applicant first identified Satb2 as a definitive marker of the presumptive large intestinal epithelium in frogs, mice, and humans. Using Satb2 as a marker, Applicant has shown that BMP signaling is required for specification of posterior gut endoderm of frogs and mice, consistent with the known role of BMP in posterior-ventral development (Kumar et al., 2003; Roberts et al., 1995; Sherwood et al., 2011; Tiso et al., 2002; Wills et al., 2008). Moreover, stimulation of BMP signaling in PSC-derived gut tube cultures for 3 days is sufficient to induce a posterior HOX code and the formation of SATB2-expressing colonic organoids. Human colonic organoids (HCOs) had a marker profile and cell types consistent with large intestine. Furthermore, HCOs, but not HIOs, formed colonic enteroendocrine cells (EEC) in response to expression of NEUROG3, demonstrating that HCOs were functionally committed to the colonic region. In addition, HCOs engrafted under the kidney capsule of immunocompromised mice and grown in vivo for 8-10 weeks, maintain their regional identify, formed tissues with colonic morphology, contained colon-specific cell types, had zones of proliferation and differentiation, as well as well-formed smooth muscle layers. Intestinal enteroids and colonoids that were derived from in vivo grown organoids maintained regional identify. Lastly, RNA-seq analysis demonstrated that HIOs and HCOs underwent substantial maturation and express regional markers consistent with a small and large intestinal identity respectively. In summary, Applicant identified an evolutionarily conserved BMP-HOX pathway in frogs and mice and used this to direct hindgut patterning and formation of human colonic organoids.

### Results

SATB2 expression marks the gut endoderm of posterior embryonic and adult intestine.

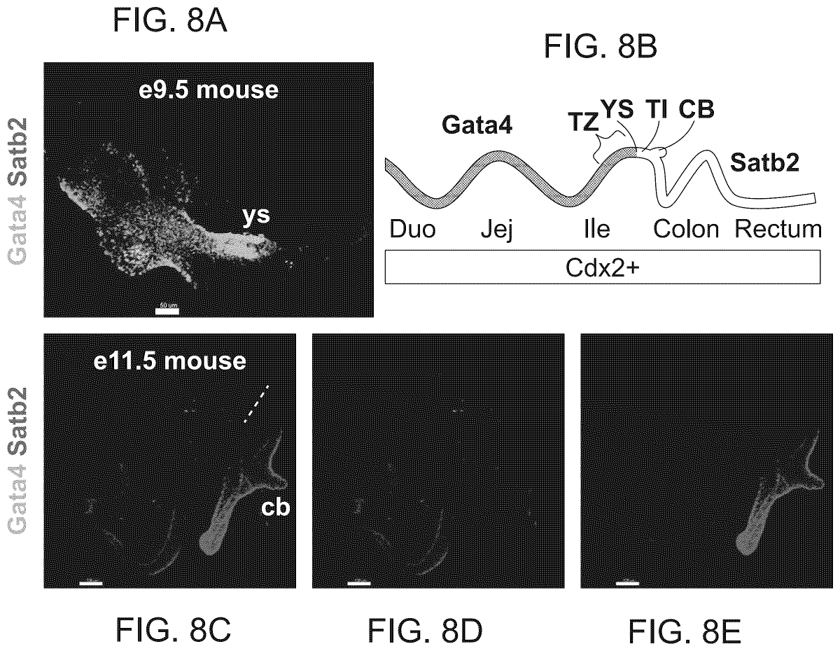

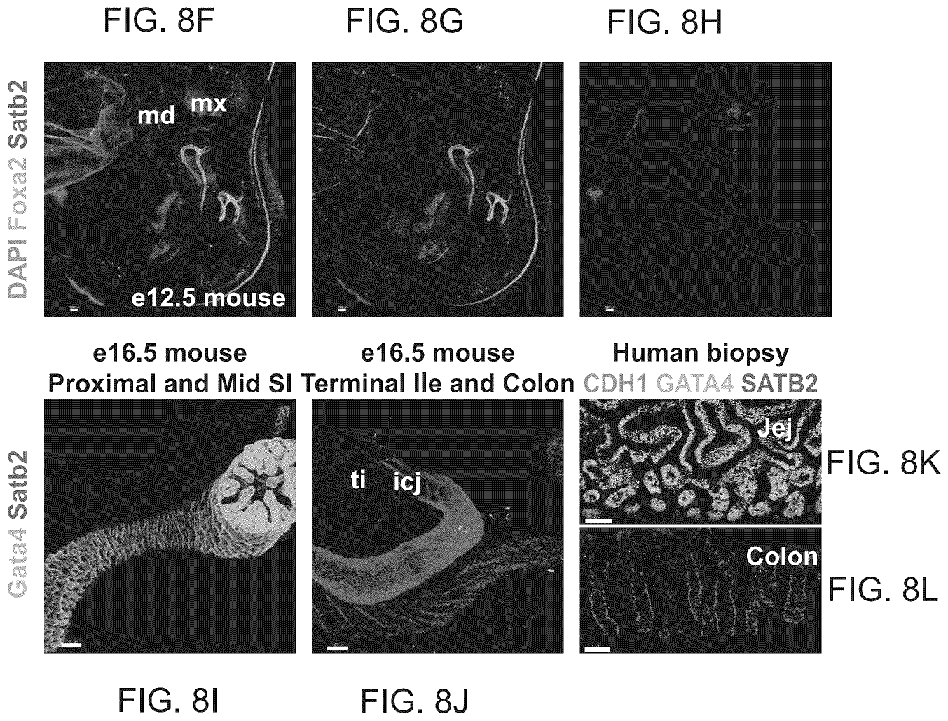

The molecular pathways that establish the mid and hindgut, the presumptive small and large intestine, are poorly understood, in part due to a paucity of well-defined markers. This has limited the ability to direct the differentiation of human PSCs into regionally distinct intestinal organoids, in particular large intestinal organoids. Applicant therefore identified markers that distinguish different domains of the mouse embryonic gut tube and used these to interrogate signaling pathways that pattern the early intestine. Consistent with previous reports Applicant found that in e9.5 mouse embryos, Gata4 marked the gut endoderm from the posterior foregut to the yolk stalk (FIG. 8A) (Aronson et al., 2014; Battle et al., 2008; Beuling et al., 2008a; Beuling et al., 2007a; Beuling et al., 2007b; Beuling et al., 2010; Beuling et al., 2008b; Bosse et al., 2007; Kohlnhofer et al., 2016; Patankar et al., 2012a; Patankar et al., 2012b; Sherwood et al., 2009; Walker et al., 2014). At later stages of development (el 1.5-e16.5), Gata4 continued to distinctly mark the anterior but not the posterior intestine (FIG. 8B-DJ-J). This expression domain remains intact into adulthood in both mice (not shown) and humans (FIG. 8K-L).

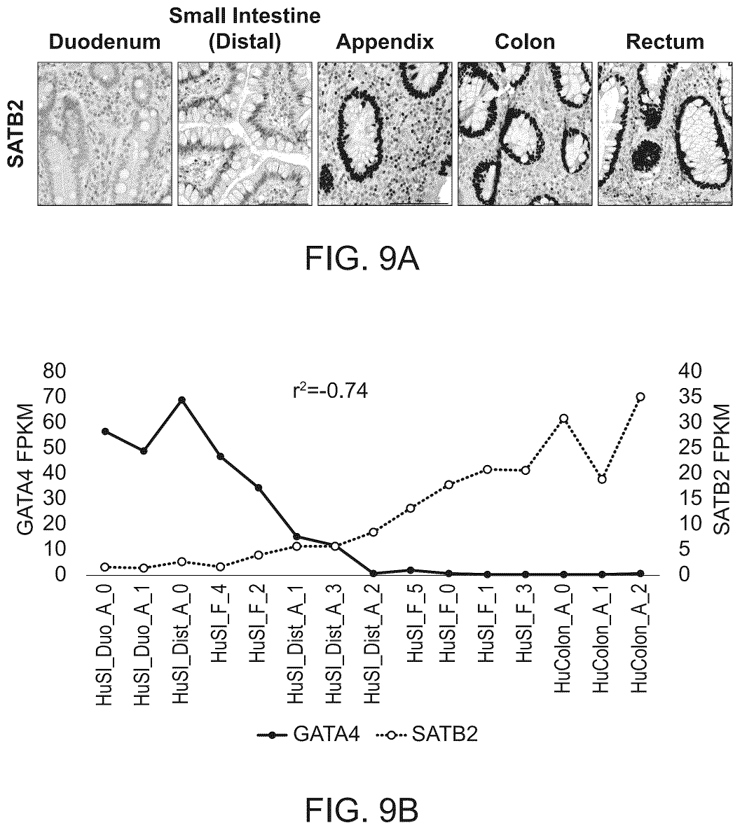

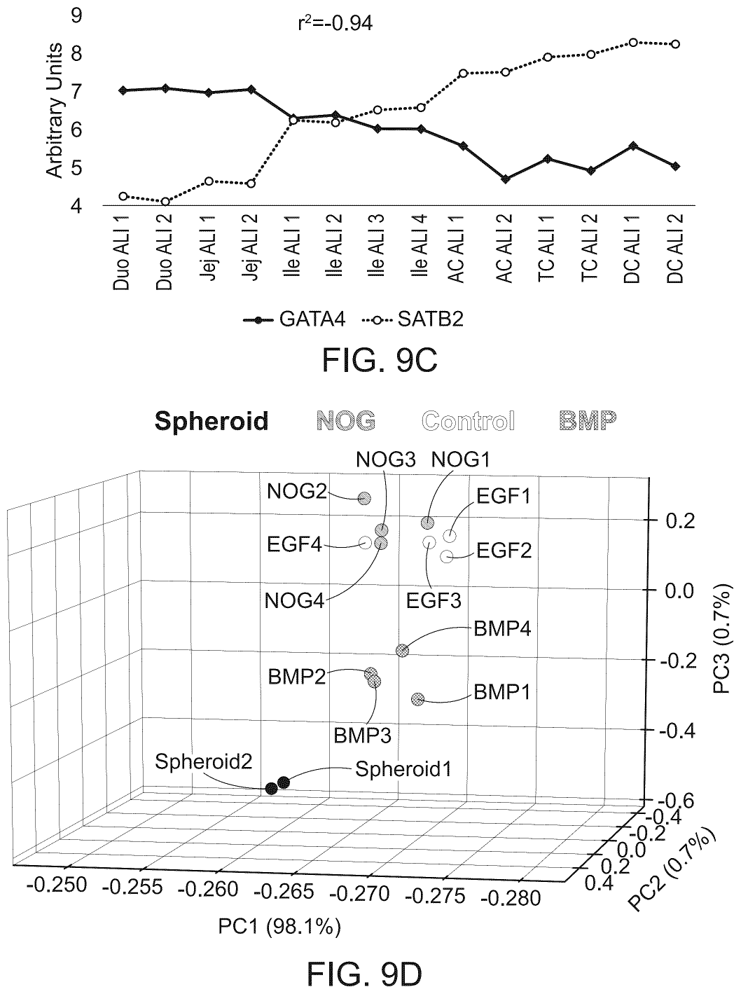

In order to identify markers of the posterior fetal intestine, Applicant mined public expression databases such as GNCPro™, TiGER and Human Protein Atlas for colon enriched genes (described in the Materials and Methods section) and found Satb2 as a potential marker of large intestine. Satb2 is a member of the CUT-class of homeobox genes (Holland et al., 2007), which binds nuclear matrix attachment regions and is involved in chromatin remodeling (Gyorgy et al., 2008). Immunostaining showed that Satb2 protein was first detected in the posterior endoderm of mouse embryos at e9-9.5 and formed a discreet expression boundary with Gata4 (FIG. 8A) at the yolk stalk, suggesting that the Satb2+ domain marks the posterior intestine, a broader expression domain than previously identified (Dobreva et al., 2006). Satb2 expression continued to mark the posterior intestinal endoderm throughout development (el 1.5-16.5) (FIG. 8 B, C, E, F, H, J) and in the postnatal colon in mice (not shown) and humans (FIG. 8L). Using published human proteome and RNA-seq data, Applicant confirmed that GATA4 and SATB2 differentially mark proximal and distal regions of the human fetal and adult intestinal tract respectively (Bernstein et al., 2010; Fagerberg et al., 2014) (Wang et al., 2015) (FIG. 9A-C). These data demonstrate that the Gata4 and Satb2 expression boundaries are established early during development of mouse and marks future boundaries of the developing small and large intestine in mice and humans.

BMP signaling is required for Satb2 expression in the embryonic hindgut endoderm.

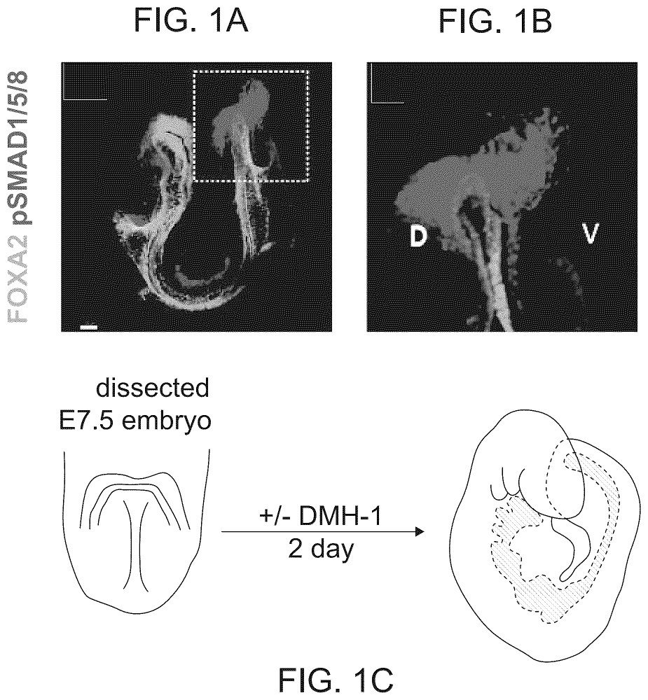

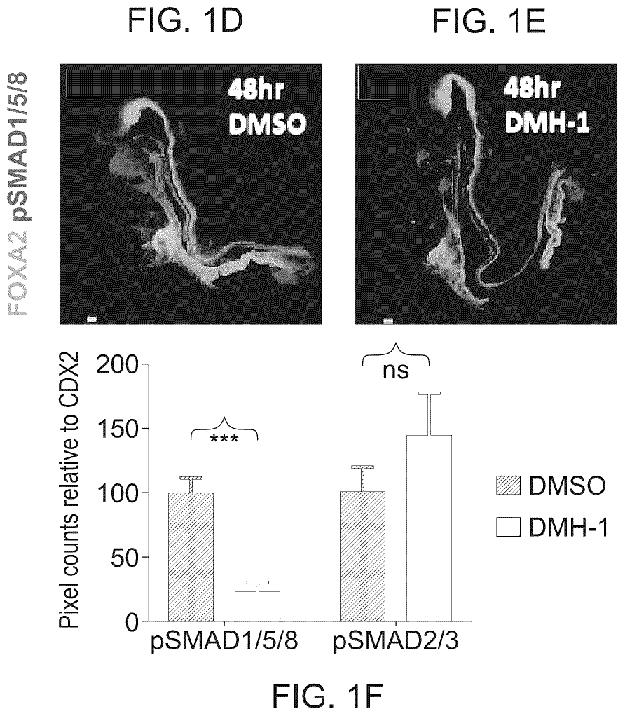

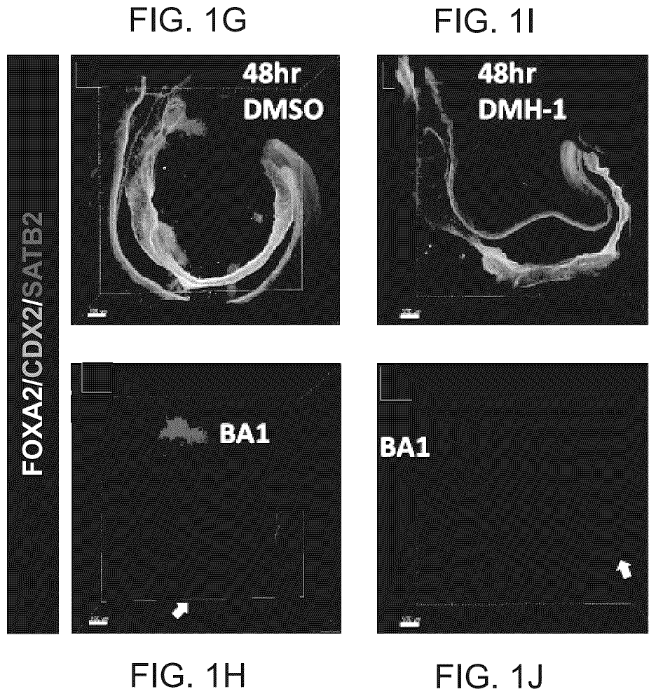

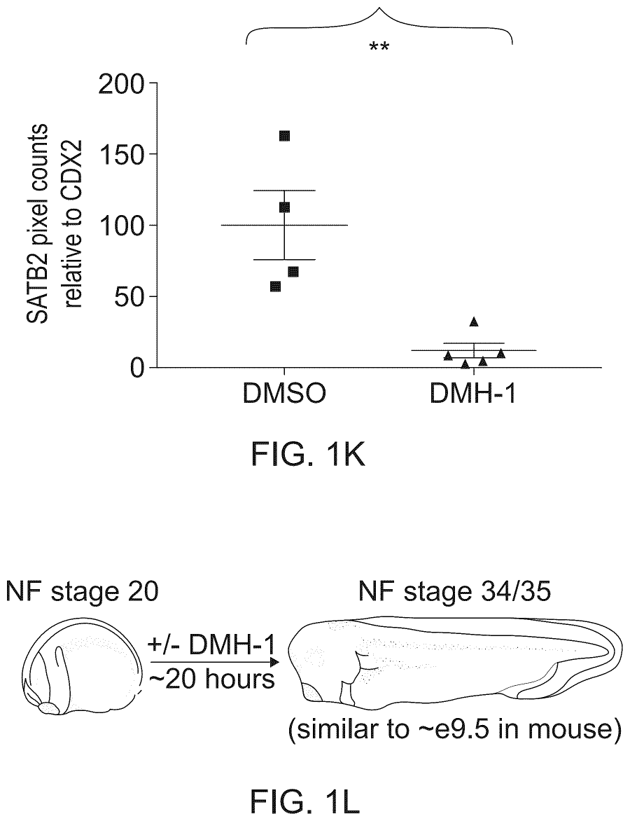

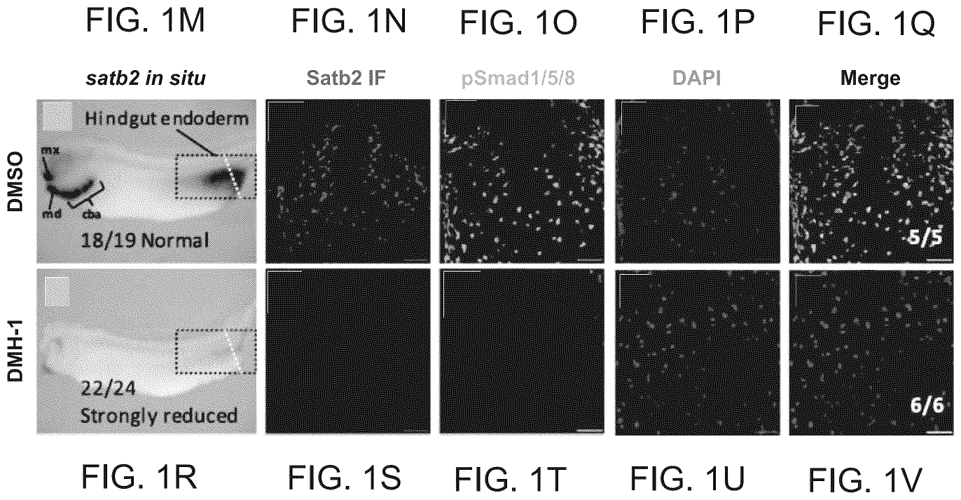

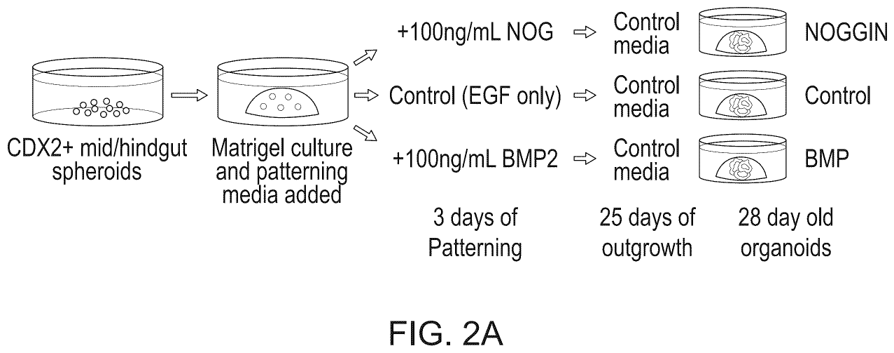

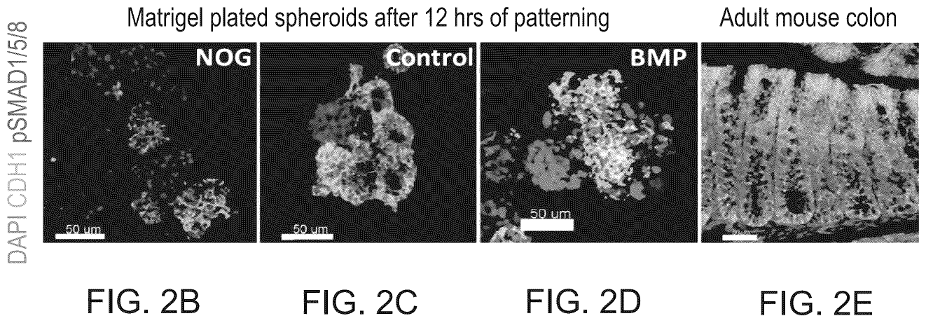

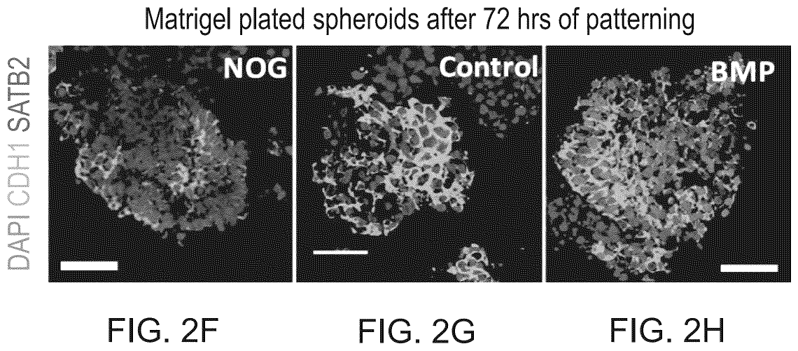

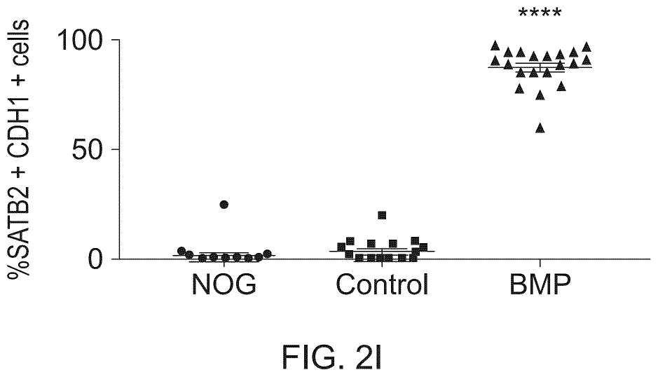

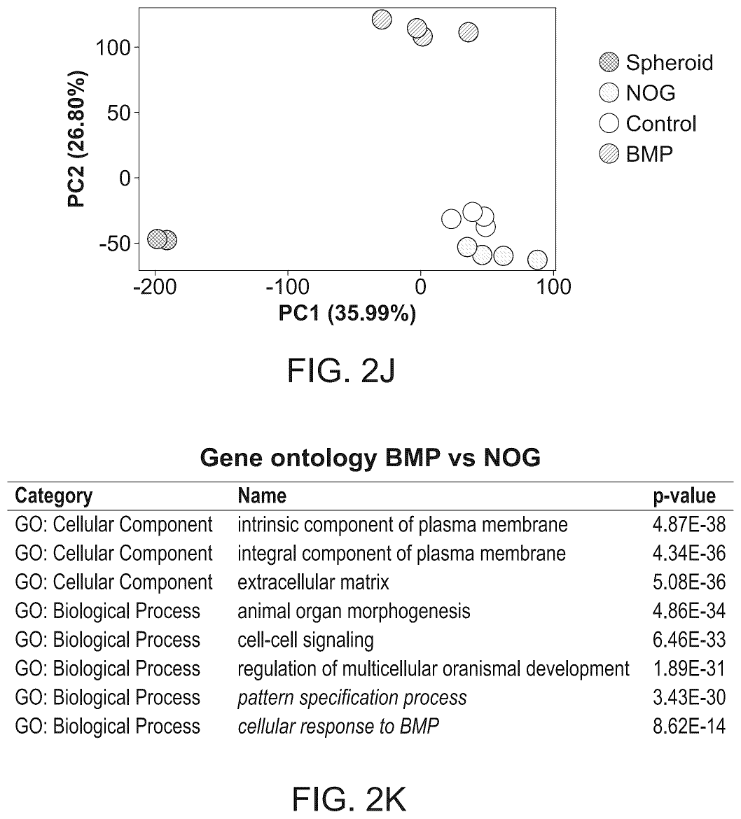

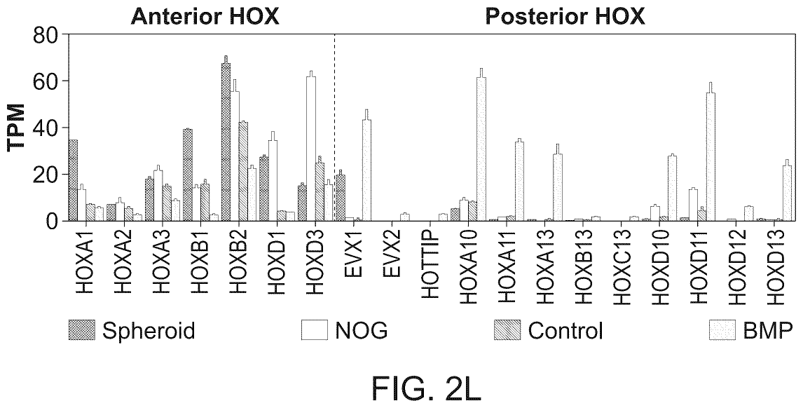

Applicant next used Satb2 as a marker to identify pathways that promote posterior intestinal fate in embryos. Applicant first determined if BMP signaling was active in the posterior gut tube, given its known role in patterning endoderm at several stages of development in zebrafish, Xenopus, chick and mouse (Kumar et al., 2003; Roberts et al., 1995; Sherwood et al., 2011; Tiso et al., 2002; Wills et al., 2008). Applicant observed that BMP signaling was highly active in the endoderm and mesoderm of the posterior gut tube of e8.5 mouse embryos as measured by phosphorylated Smad1/5/8 (pSMADI/5/8) (FIG. 1A-B). To determine if BMP signaling is required for patterning of the posterior gut tube, Applicant cultured early headfold stage mouse embryos (e7.5) in the BMP signaling inhibitor DMH-1 (FIG. 1C). After 48 hours of DMH-1 treatment, Applicant saw a significant reduction in pSmad1/5/8 levels and a loss of Satb2 expression in the posterior gut tube (FIG. 2D-K). In addition, Satb2 expression was lost in the first brachial arch of DMH-1 treated embryos consistent with previous studies in Zebrafish (Sheehan-Rooney et al., 2013). DMH-1 had no impact on TGFI3 signaling as measured by pSmad2/3 levels (FIG. 1F). Given the evolutionary conservation of the Satb2 across vertebrate species (Li et al., 2006) Applicant investigated if BMP is required for Satb2 expression in the hindgut of frog embryos (FIG. 2L). Similar to mice, treatment of Xenopus embryos with DMH-1 (FIG. 1M-V), or transgenic expression of the BMP-antagonist Noggin (not shown) resulted in a loss of Satb2 expression in the hindgut and brachial arches. BMP signaling has been shown to directly regulate Satb2 expression in mouse embryonic mandibles through direct binding of Smad1/5 to a conserved enhancer (Bonilla-Claudio et al., 2012), suggesting that Satb2 may be a direct BMP target in the gut as well. Taken together these results revealed a conserved pathway in vertebrates whereby BMP signaling is required for defining the posterior most region of the developing gut tube that gives rise to the distal ileum and large intestine.

BMP signaling promotes posterior fate in human gut tube cultures.

Applicant next investigated if BMP signaling could be used to promote a posterior gut tube fate in humans using nascent CDX2+ gut tube spheroids derived from human PSCs as previously described (Spence et al., 2011). Applicant either inhibited or activated BMP signaling using the BMP inhibitor NOGGIN or BMP2 respectively (FIG. 2A) and monitored BMP signaling levels by accumulation of nuclear pSMADI/5/8. Control cultures had low levels of pSMADI/5/8 protein and addition of NOGGIN abolished this staining (FIG. 2B-D). In contrast, addition of BMP2 caused a rapid accumulation of pSMAD158 in both epithelial and mesodermal cells suggesting both cell types respond to BMP signals similar to what Applicant observed in mouse embryos (FIG. 1A-B). The specificity of pSmad1/5/8 staining was confirmed using adult mouse colon, which showed pSmad 1/5/8 staining restricted to the differentiated compartment of the upper crypt (FIG. 2E) as previously reported (Hardwick et al., 2004; van Dop et al., 2009; Whissell et al., 2014). Further analysis of organoids revealed that 3 days of BMP2 treatment was sufficient to induce high levels of SATB2 protein in the epithelium compared to NOGGIN and control cultures (FIG. 2F-I). This suggests that a short pulse of BMP activity is sufficient to pattern spheroid endoderm into a posterior gut tube fate.

While BMP signaling is known to regulate anterior-posterior patterning of the endoderm, little is known about the transcriptional networks that ultimately confer positional identity along the A-P axis in mammals. Applicant used human gut tube spheroids and RNA-seq to identify how BMP signaling establishes posterior domains in the developing human gut. Principal component analysis revealed that gut tube spheroids treated with BMP for 3 days clustered separately from NOGGIN and control treated organoids (FIG. 2J). Examination of gene ontology terms (GO terms) revealed that modulation of BMP signaling affects multiple biological processes including organ morphogenesis, cell-cell signaling, pattern specification and cellular response to BMP signaling (FIG. 2K). The most definitive regulators of A-P patterning are HOX genes, and Applicant found that BMP activation resulted in down regulation of anterior HOX genes and up regulation of posterior HOX genes (FIG. 2L). In particular Applicant saw BMP-mediated increases in multiple paralogs of HOX10, 11, 12 and 13 groups. These results demonstrate that BMP signaling broadly regulates A-P hox code during patterning of the human gut tube and suggest a mechanism by which the distal GI tract is initially specified.

BMP signaling acts downstream of SHH to induce a posterior HOX code.

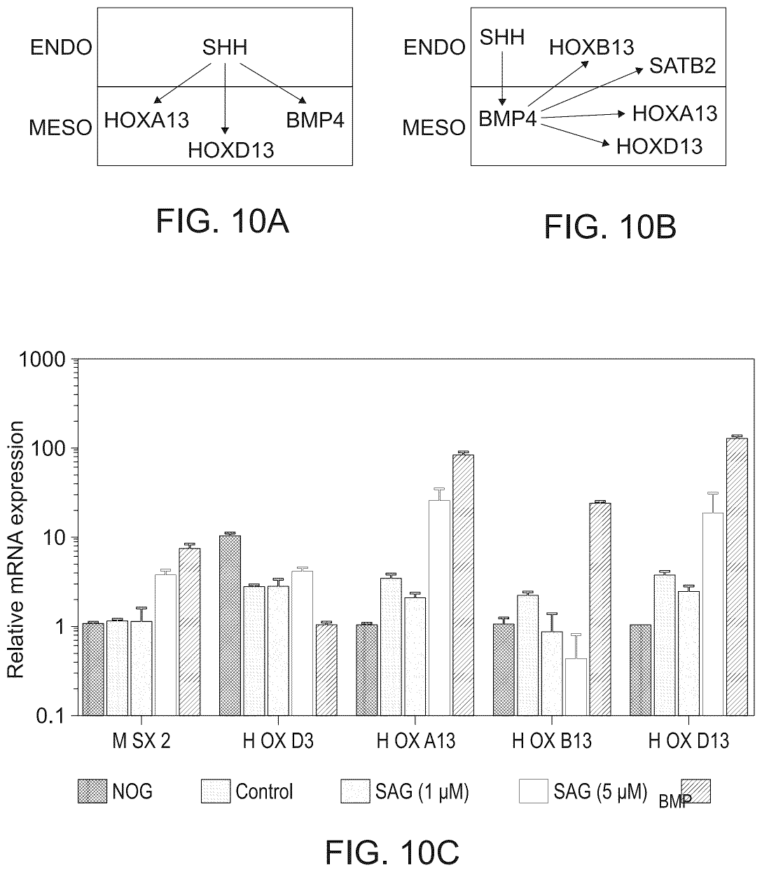

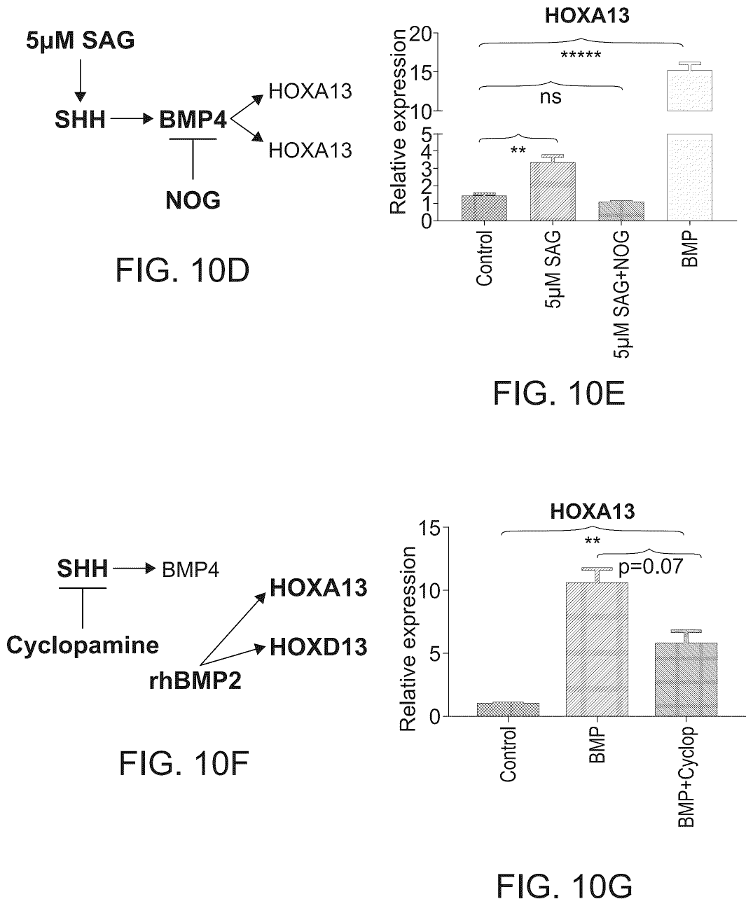

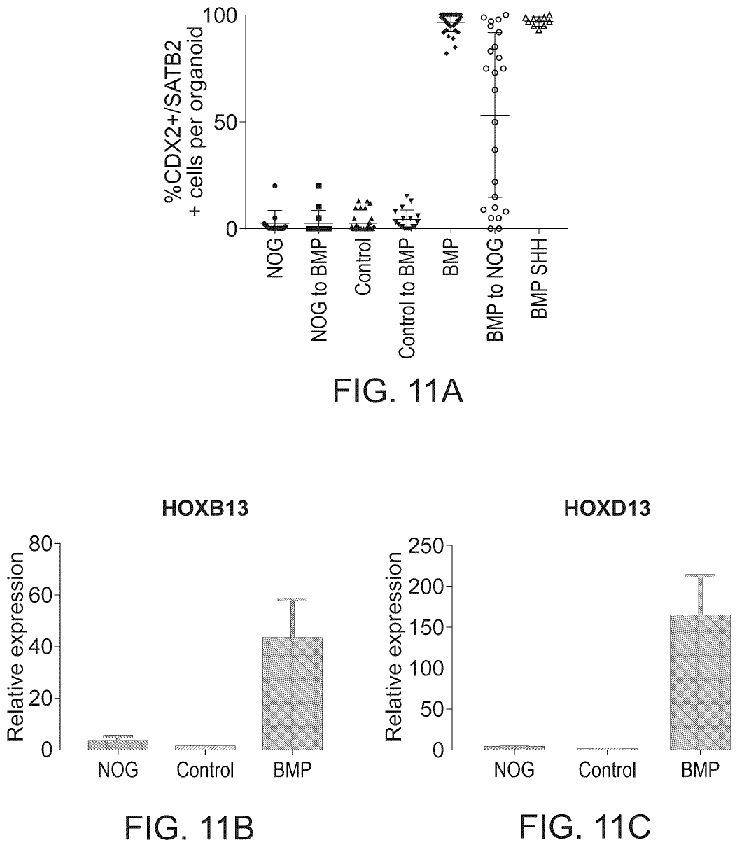

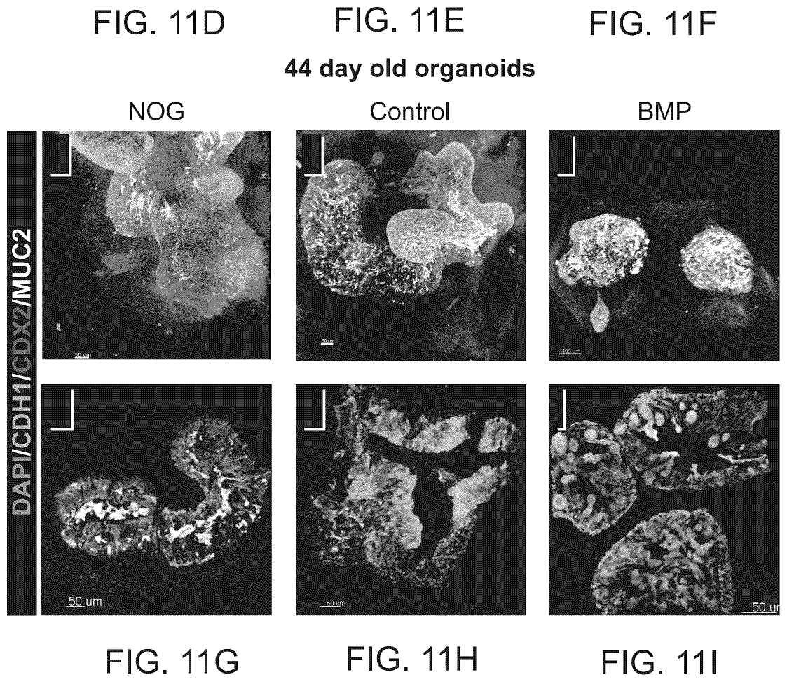

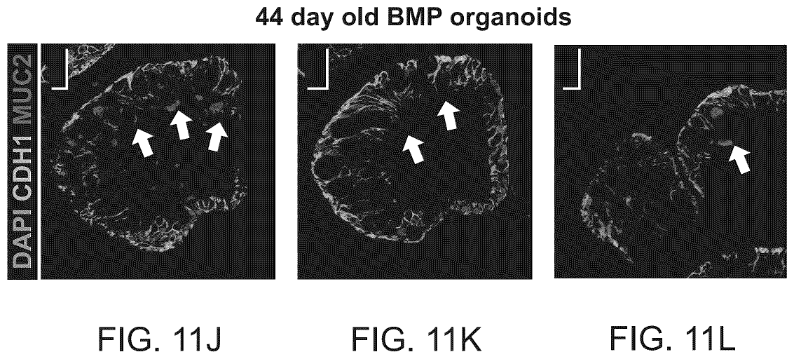

Previous studies suggest that Sonic Hedgehog (Shh) acts upstream of Bmp4 and Hoxl3 expression during posterior gut patterning in chick embryos (FIG. 10A) (Roberts et al., 1995). However, the relative epistatic relationship between BMP and Hoxl3 (FIG. 10B) was not investigated due to embryonic lethality caused by Bmp4 overexpression in the midgut and hindgut (De Santa Barbara et al., 2005; Roberts et al., 1995). Applicant used human gut tube cultures to better model the epistatic relationship of SHH-BMP-HOX13 during posterior gut tube patterning. Activation of hedgehog signaling with the smoothened agonist SAG led to a concentration dependent activation of the BMP signaling target gene MSX2 and the mesenchymal HOX factors, HOXA13 and HOXD13 (FIG. 10C). However, SAG-mediated activation of these factors was only a fraction of the activation mediated by BMP2 (FIG. 10C). Applicant further showed that the ability of HH signaling to activate HOXA13 was entirely dependent on BMP (FIG. 10D-E), confirming that BMP signaling functions downstream of SHH as previously reported (Shyer et al., 2015; Walton et al., 2012; Walton et al., 2009; Walton et al., 2016). It has not been determined if BMP signaling is sufficient to activate the posterior HOX program downstream of HH signaling. Applicant therefore examined HOXA13 induction by BMP in the presence of the SHH inhibitor Cyclopamine and found that BMP2 was sufficient to induce HOXA13 when SHH signaling is inhibited (FIG. 10F-G). Consistent with this, activation of SHH signaling during BMP patterning did not improve SATB2 expression (FIG. 11A). Experiments in Xenopus confirmed this epistatic relationship between SHH and BMP (data not shown) suggesting that this mechanism is evolutionarily conserved. Taken together Applicant's data suggest that BMP signaling is sufficient to activate the posterior HOX code and does so downstream of HH signaling.

BMP-derived organoids cultured in vitro maintain a distal identity.

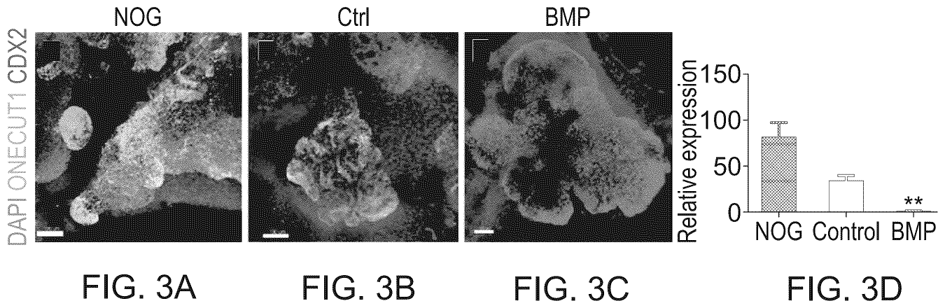

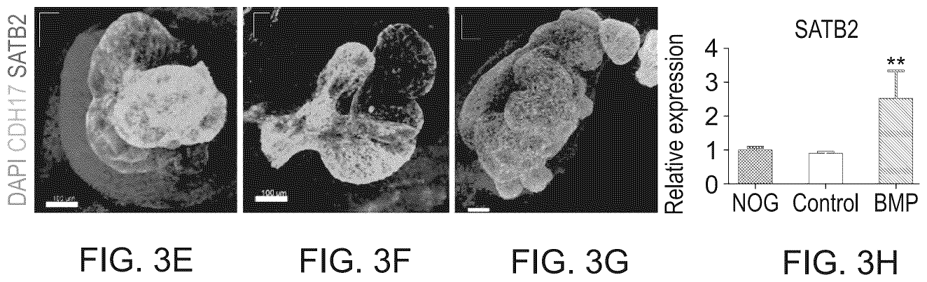

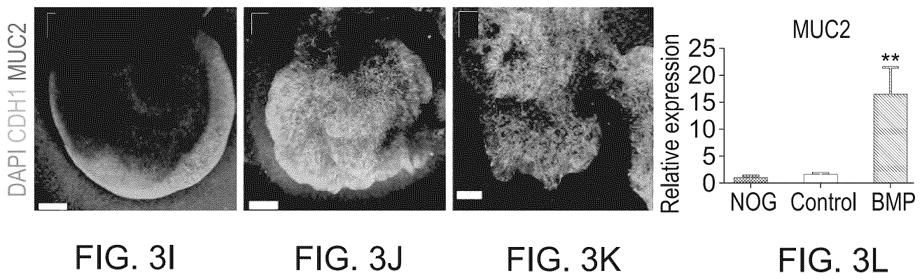

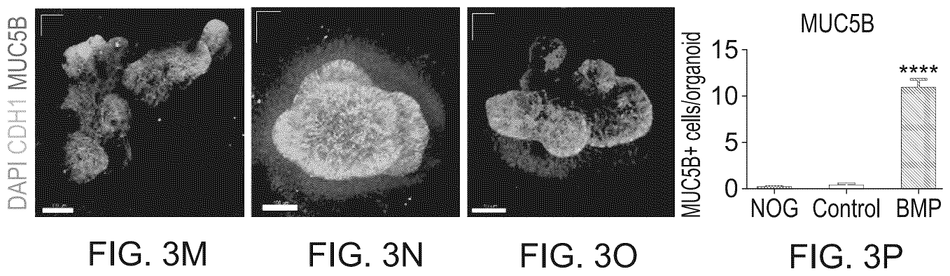

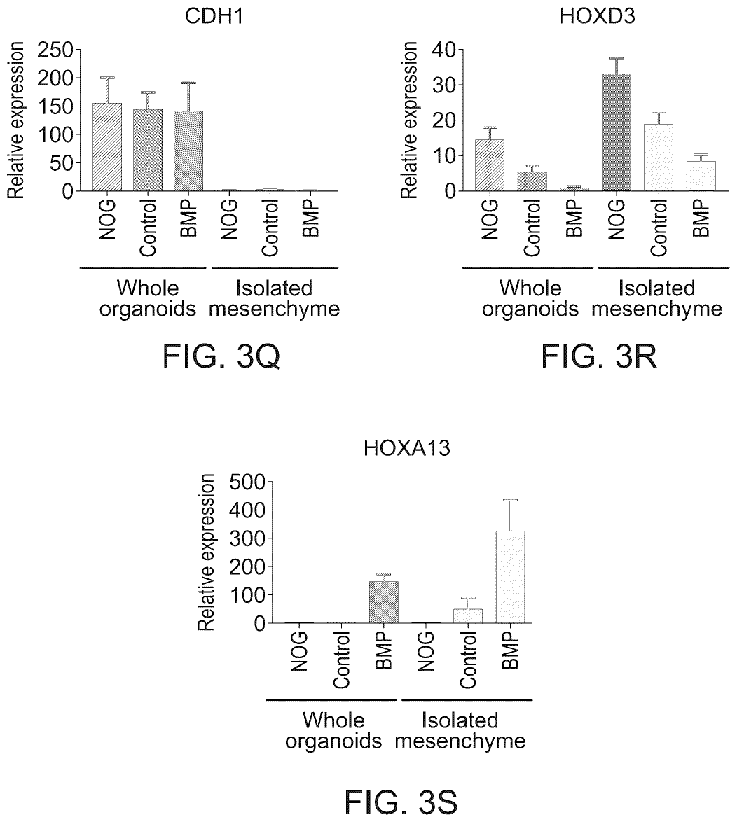

Applicant next investigated if 3 days of BMP treatment is sufficient to confer stable regional identity following extended culture of organoids for 25 days (FIG. 3). Levels of ONECUT1 (a marker of proximal small intestine) were highest in NOGGIN and control treated organoids and absent in BMP2 treated organoids (FIG. 3A-D). Conversely, SATB2 was absent in the epithelium of NOGGIN and control treated organoids but broadly expressed in nearly all of the CDX2+ epithelial cells of BMP2 treated organoids (FIG. 3 E-H, FIG. 11 A). Importantly, modulation of BMP signaling had similar proximal-distal patterning effects on multiple human PSC lines, including embryonic stem cell lines HI and H9 and induced pluripotent stem cell lines (IPSC 54.1 and IPSC 72.3) (shown below). Applicant frequently observed non-epithelial SATB2 expression in NOGGIN and control organoids (data not shown) possibly due to the presence of other cell types that are known to be present in HIOs in vitro (Spence et al., 2011). Examination of HOXB13 and HOXD13, which is expressed in posterior epithelium and mesenchyme respectively, further revealed that BMP treated organoids maintained posterior patterning following prolonged culture in vitro (FIG. 11B-C).

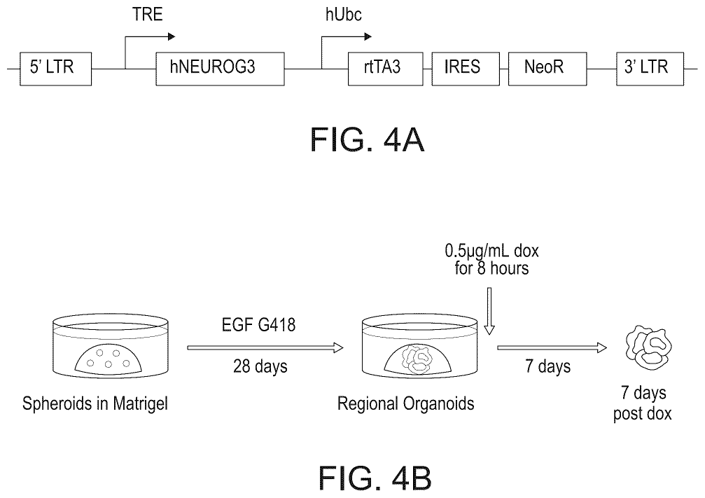

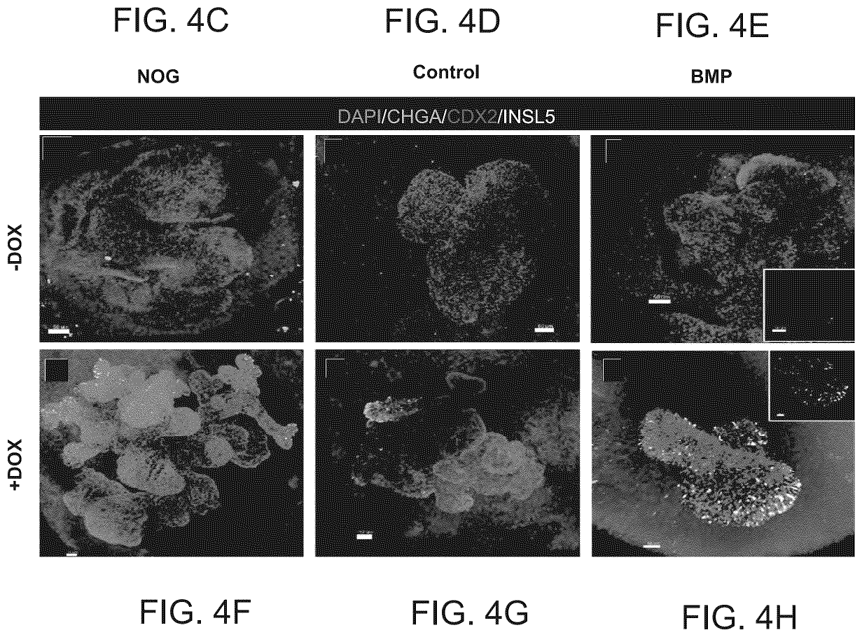

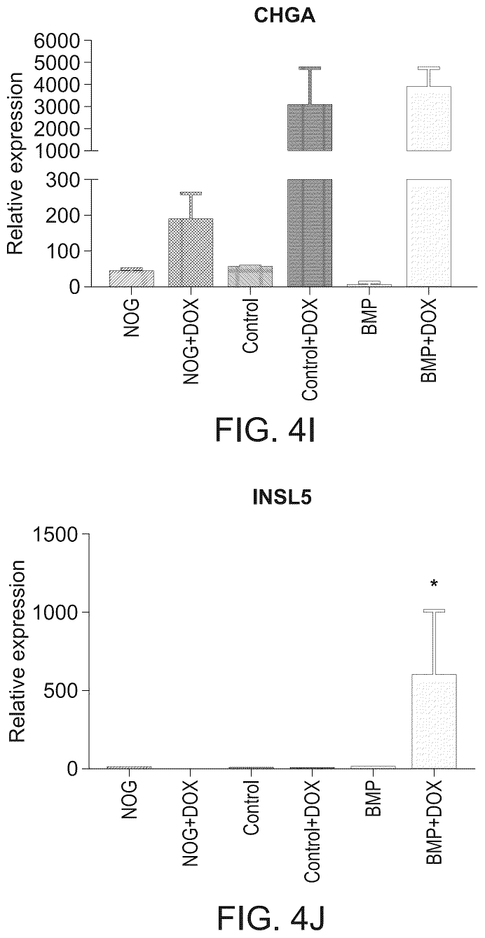

Goblet cells are distributed in a low-to-high gradient from proximal small intestine to distal large intestine (Rodriguez-Pineiro et al., 2013), and Applicant investigated if goblet cell numbers were lower in proximal and higher in distal organoids. Analysis of MUC2 staining at 28 days revealed that BMP2 treated organoids had high numbers of goblet cells as visualized by intracellular MUC2 (FIG. 3I-L) as compared to more proximal NOGGIN treated and control organoids, which only had rare intracellular MUC2 staining. Applicant further confirmed the regional identity of goblet cells using the marker MUCSB, which is expressed by a subset of goblet cells in the colon but not in the small intestine (van Klinken et al., 1998). MUC5B staining was absent in Noggin and control treated 28-day organoids but was present in BMP2 treated organoids (FIG. 4M-P). Goblet cell morphology became more mature in older organoids (FIG. 11D-I), where in 44-day old BMP treated organoids Applicant observed goblet cells in the process of secreting mucus into the lumen of the organoids (FIG. 11J-L). The ability to observe mucus secretion in BMP treated organoids suggests that this organoid system would be useful to study mucus secretion and the roles of mucus in intestinal pathophysiology.

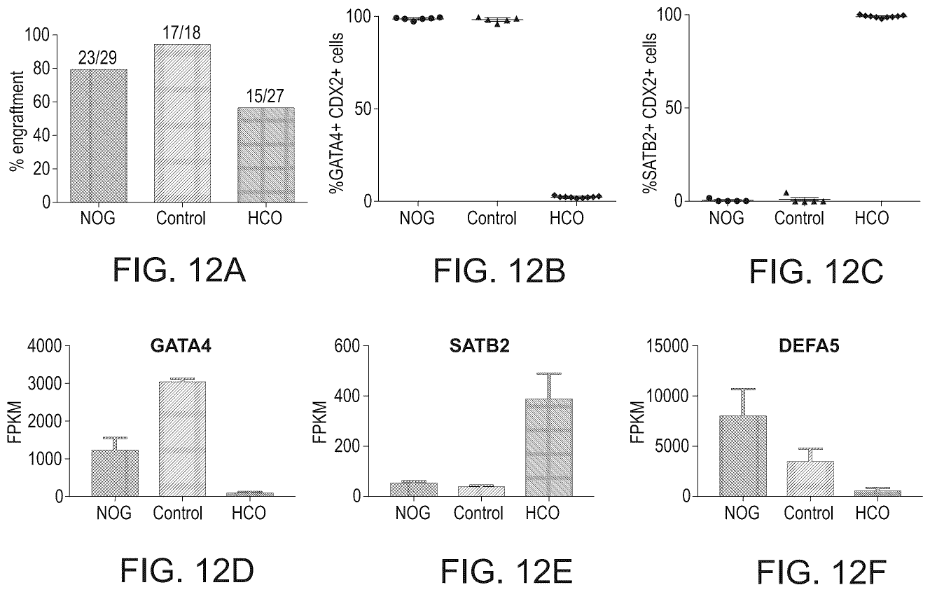

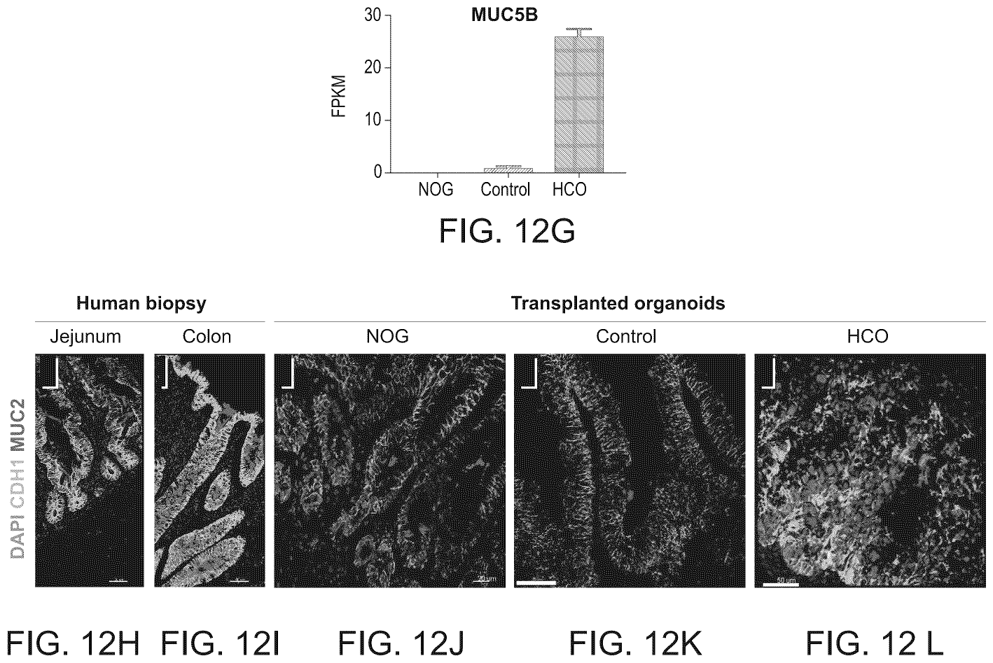

While the regional pattern of organoids is stable after 28 days in culture, Applicant wanted to investigate if early patterning was fully established after the initial 3-day treatment. To do so, Applicant shifted 3-day NOGGIN-treated spheroids to BMP2-containing media for 3 days and conversely shifted 3-day BMP treated spheroids to NOGGIN-containing media for 3 days. Proximal organoids generated with NOGGIN did not express SATB2 in response to BMP2 demonstrating that proximal fate was stable following 3 days of patterning (FIG. 11 A). In the converse experiment, while 3 days of BMP2 treatment was sufficient to induce a stable distal fate, a subset of organoids lost SATB2 expression in response to NOGGIN treatment (FIG. 11 A). While 3 days of BMP2 treatment is sufficient to induce a colonic fate that is stable in vitro and in vivo (FIG. 12), there remains plasticity in the early posterior gut tube. This is consistent with the observation that the colonic endoderm of midgestation rat embryos is more regionally plastic than the small intestinal endoderm (Ratineau et al., 2003).

Patterning of organoid mesenchyme by BMP signaling.

While stimulation of BMP signaling conferred regional identity to organoid epithelium, Applicant also observed pSMAD 1/5/8 in the non-epithelial compartment of BMP2 treated organoids during patterning, and upregulation of posterior HOX factors known to be expressed in the mesenchyme. To determine if mesenchymal patterning was stable, or required continued patterning input from epithelium, Applicant isolated and expanded mesenchymal cell cultures for 2-3 weeks and analyzed them for expression of regional HOX genes. Mesenchymal cultures were lacking E-cadherin expressing cells, suggesting that they were primarily comprised of mesenchyme (FIG. 3Q). Analysis of HOXD3, which is enriched in proximal intestinal mesenchyme (Yahagi et al., 2004), confirmed that the mesenchyme from NOGGIN and control treated organoids have a stable proximal identity while BMP treated organoids had decreased expression of HOXD3 (FIG. 3R) and high levels HOXA13 (FIG. 3S), which continues to be expressed in human colon fibroblasts (Higuchi et al., 2015). Taken together, these data suggest that early modulation of BMP signaling patterns both the epithelium and the mesenchyme and that mesenchymal patterning is stable even in the absence of epithelium.

Induction of colonic enteroendocrine cells is restricted to BMP2 treated organoids.

The development of several ECC subtypes is regionally restricted to specific segments of small and large intestine. For example, expression of the protein INSL5 is restricted to colonic EECs (Burnicka-Turek et al., 2012; Thanasupawat et al., 2013). As a functional test of colonic identity, Applicant determined if experimental induction of the colonic EEC marker INSL5 was restricted to BMP2-treated distal organoids. To do this Applicant inducibly expressed the proendocrine transcription factor NEUROG3 using an iPSC line harboring a doxycycline (DOX) inducible NEUROG3 expression cassette (FIG. 4 A) as previously described (McCracken et al., 2017; McCracken et al., 2014). Applicant performed a 6-hour pulse of DOX and after an additional 7 days in culture observed a robust induction of EECs as measured by CHGA positive cells (FIG. 4B-I). However, Applicant only observed INSL5 positive cells in BMP2 treated organoids and confirmed this by QPCR analysis (FIG. 4C-HJ). Given that INSL5-expressing cells are only in the colon, Applicant's data strongly suggest that BMP2-treated organoids are functionally committed to the colonic fate. The expression of distal markers like SATB2, MUC5B and HOXA13 and the competence to generate colon specific ECCs support the conclusion that BMP2 treated organoids are colonic, and thus will be referred to as human colonic organoids (HCOs).

Regional identity of patterned organoids is maintained in vivo.

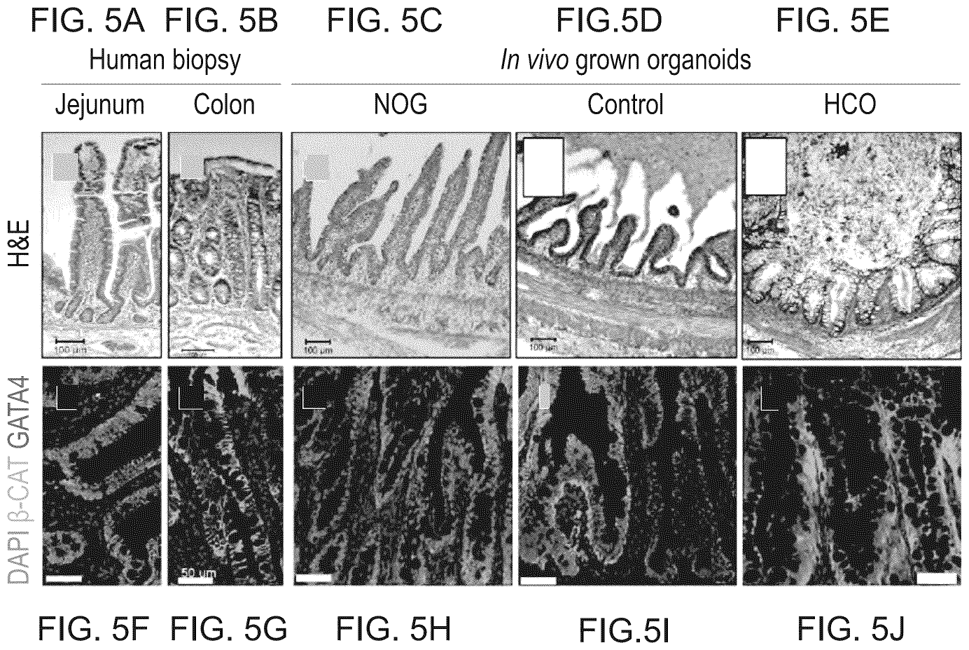

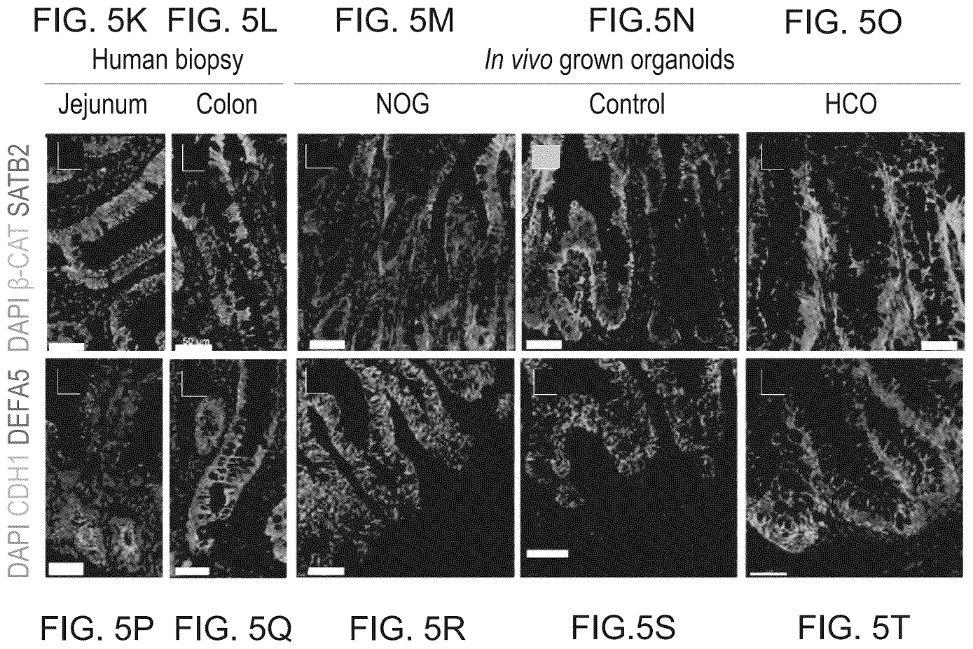

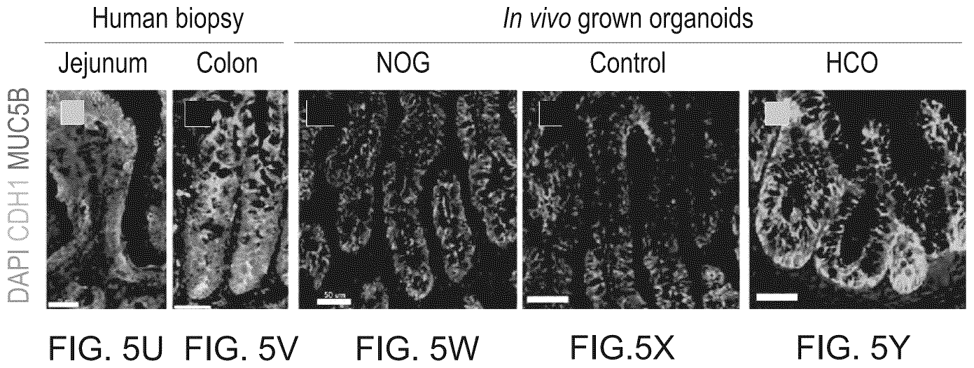

Previous studies of mouse and human fetal intestine have demonstrated that regional identity and tissue morphology of different regions of the intestine were maintained following orthotopic transplantation and growth in immunocompromised mice (Duluc et al., 1994; Savidge et al., 1995). To determine if HIOs and HCOs that were patterned in vitro would maintain regional identity and grow into small and large intestinal tissue, Applicant transplanted them under the mouse kidney capsule for 6-10 weeks, which Applicant previously demonstrated results in HIO maturation into small intestinal tissue (Watson et al., 2014). Applicant observed that the engraftment of NOGGIN and control HIOs was more efficient than HCOs (FIG. 12A). Consistent with their regional identity, transplanted HIOs and HCOs developed into mature tissues that morphologically resembled either small or large intestine, respectively (FIG. 5A-E). The epithelium of NOGGIN and control organoids formed well-defined crypts and tall villi, comparable to human small intestine. In contrast BMP2-treated organoids contained crypts but lacked villi, similar to colon.

In addition to their morphological resemblance to either small or large intestine, transplanted HIOs and HCOs expressed distinct regional markers and contained regionally enriched cell types. For example, the majority of the epithelium of NOGGIN and control HIOs expressed the proximal marker GATA4 and did not express the large intestinal marker SATB2 (FIG. 5F-I, K-N, FIG. 12B-E). Conversely HCO epithelia were uniformly SATB2+ but did not express GATA4 (FIG. 5J,0, FIG. 12B-E). In addition, Paneth cells expressing DEFA5 were present in the crypts of NOGGIN and control HIOs, but were absent HCOs (FIG. 5P-T, FIG. 12F) similar to the human colon (Wehkamp et al., 2006). Applicant further confirmed the colonic identity of HCOs using the colonic goblet cell marker MUC5B (van Klinken et al., 1998), which is expressed by a subset of goblet cells of HCOs but is not detectible in NOGGIN or control HIOs (FIG. 5U-Y, FIG. 12G). Additionally, the number of MUC2+ goblet cells was vastly higher in HCOs compared HIOs consistent with the abundance of goblet cells seen in the human colon (FIG. 12H-L). The patterning markers, the presence of MUC5B-expressing goblet cells, and the absence of Paneth cells all support the conclusion that transplanted HCOs have colonic epithelium.

In vivo matured HIOs and HCOs express regional enteroendocrine hormones.

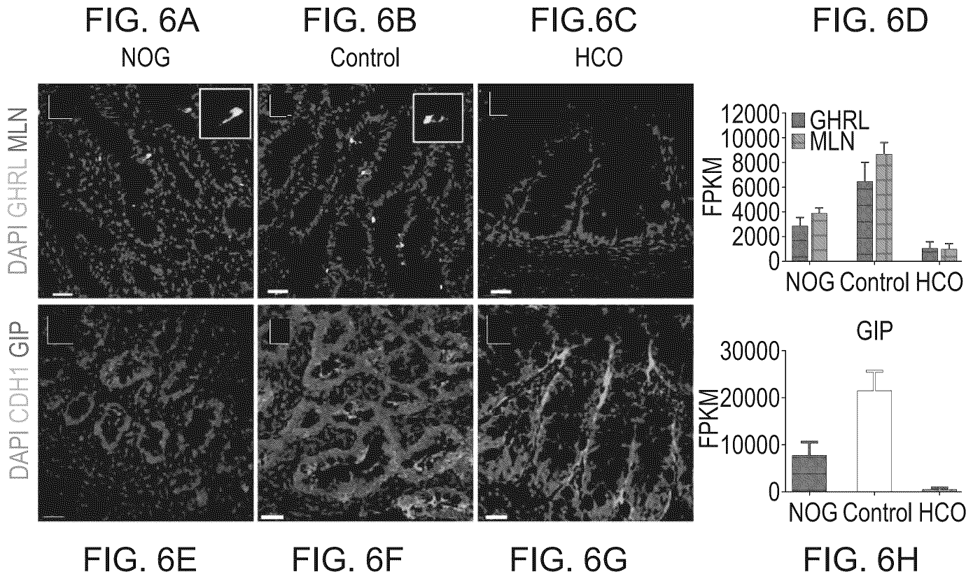

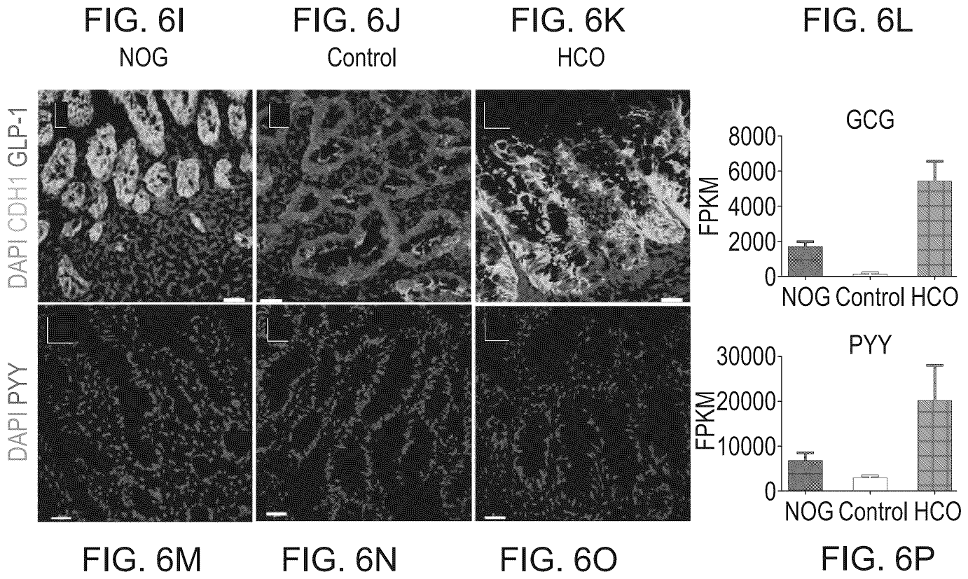

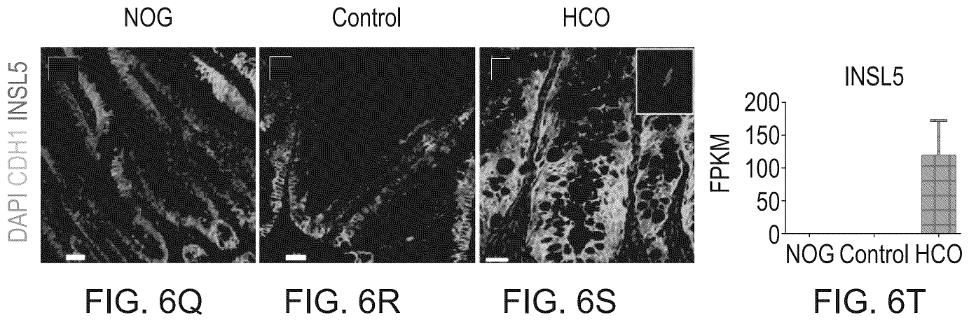

There are at least 12 major EEC subtypes that are found in different regions of the gastrointestinal tract and Applicant analyzed HIOs and HCOs for the presence of regional EECs. Ghrelin and Motilin are found predominantly in the proximal intestine, and correspondingly these hormones were largely expressed in NOGGIN and control HIOs but not HCOs (FIG. 6A-D). Similarly, GIP, which is found in K-cells of the small intestine but is absent in the colon, were found in NOGGIN and control HIOs but not in HCOs (FIG. 6E-H). Applicant then examined presence of distally enriched EECs in HCOs by analyzing for expression of GLP-1 and PYY, which are more abundant in the colon. Applicant observed higher numbers of GLP-1 and PYY cells and higher expression of preproglucagon and PYY in HCOs than in HIOs (FIG. 61-P). In addition, Applicant found expression of the colon specific hormone INSL5 (Burnicka-Turek et al., 2012; Thanasupawat et al., 2013), only in HCOs (FIG. 6Q-T).

Analysis of stem and progenitor cells in HIOs and HCOs in vitro and in vivo.

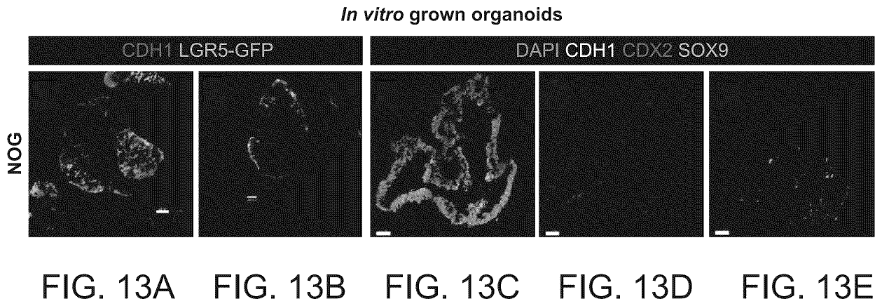

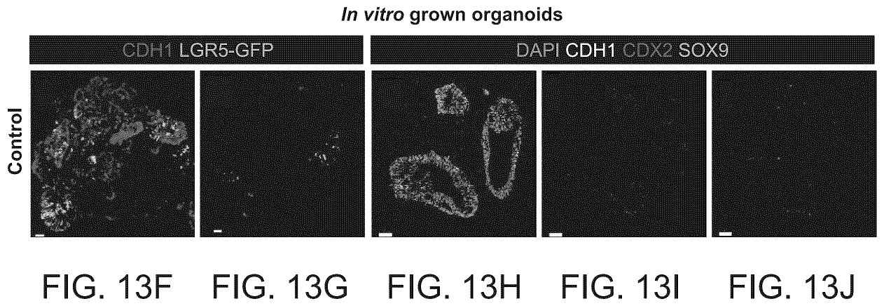

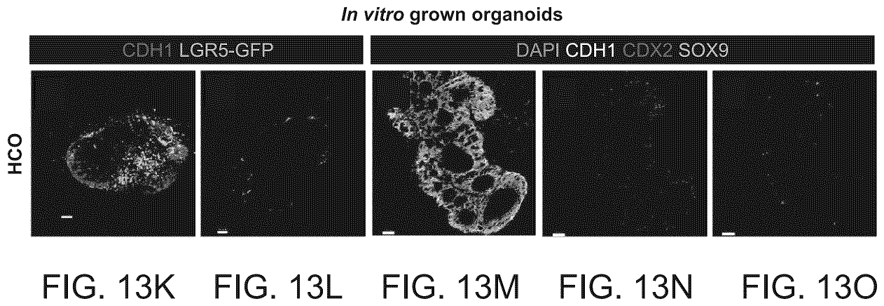

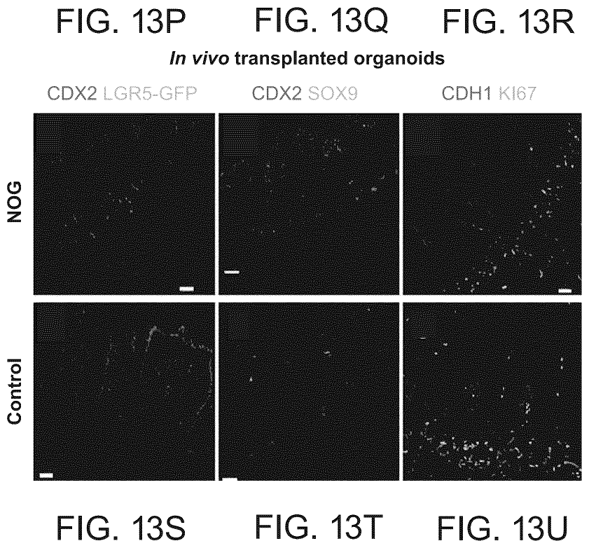

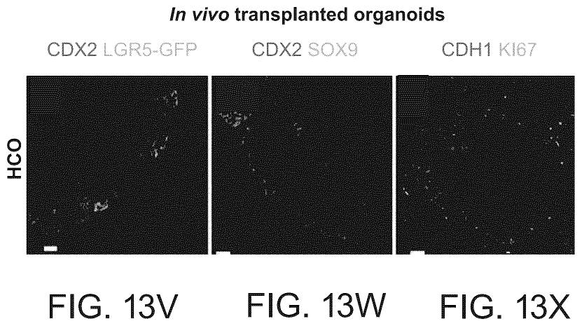

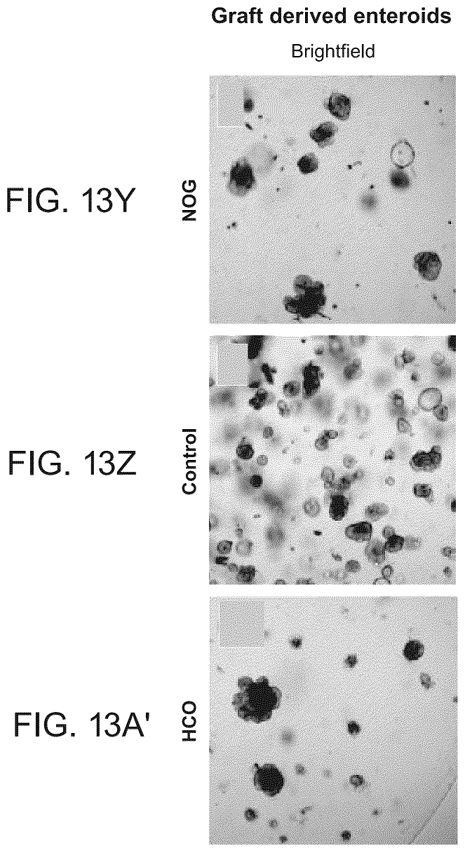

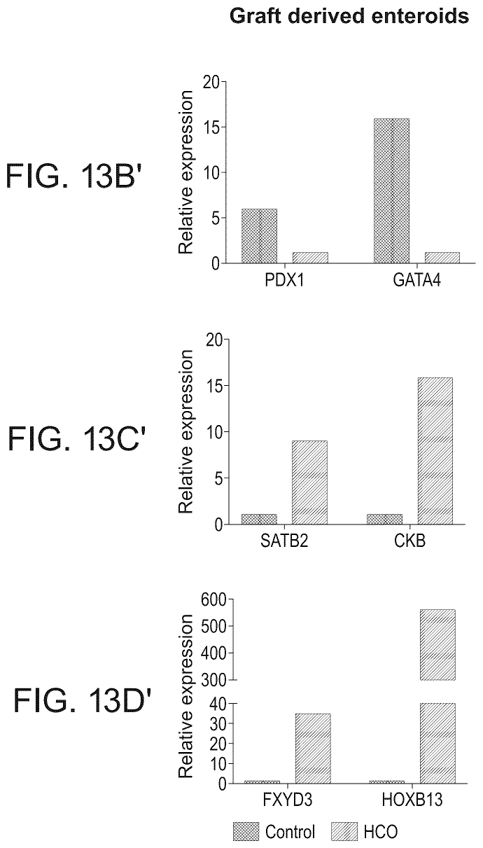

To determine if in vitro-derived HIOs and HCOs express markers of stem and progenitor cells, Applicant used the H9-BAC-LGR5-eGFP transgenic line that has been described previously (McCracken et al., 2014; Watson et al., 2014). Examination of LGR5-eGFP expression in organoids revealed expression in broad epithelial domains similar to the expression patterns in Lgr5-eGFP mice as early as e13.5 (Shyer et al., 2015) (FIG. 13 A, B, F, G, K, L). GFP expression was also evident outside the epithelium of organoids as determined by histology and FACS analysis which revealed a population of GFP+ EPCAM-cells (data not shown). In addition, Applicant examined the expression of SOX9, which is a marker of progenitor cells in the fetal and adult intestine and found it expressed in the epithelium of both HIOs and HCOs (FIG. 13C-E, H-J, M-0). These data suggest that embryonic/fetal intestinal progenitor cells, marked by LGR5-eGFP and SOX9, are present in HIOs and HCOs in vitro.

At later stages of intestinal development, progenitor cells become restricted to the base of developing villi, where they will eventually contribute to the intestinal stem cells (ISCs) of the crypts of Lieberkuhn. To determine if the progenitor cells that Applicant observed in vitro will undergo this this developmental transition, Applicant transplanted HIOs and HCOs and monitored LGR5-eGFP, SOX9, and KI67 protein. Following maturation of organoids in vivo, Applicant observed LGR5-eGFP, SOX9, and KI67 restricted to the base presumptive crypts (FIG. 13P-X). In addition, SOX9 was also observed in EECs in the villi of HIOs and in the cuff of the colonic epithelium transplanted HCOs consistent with SOX9 expression in these cell types. Given that Sox9 and Lgr5 mark intestinal and colonic stem cells capable of forming enteroids and colonoids in mice (Gracz et al., 2010; Ramalingam et al., 2012) Applicant investigated if the epithelium of transplanted organoids could be isolated and used to generate enteroids and colonoids. Both HIOs and HCOs gave rise to cultures of epithelial organoids that grew and could be passaged (FIG. 13Y-A′). Moreover, HCO-derived epithelial cultures expressed the colonic markers CKB, FXYD3, SATB2, and HOXB13 but did not express the proximal small intestine markers PDX1 or GATA4 suggesting that regional identity was maintained (FIG. 13B‘-D’). These data suggest that HIOs and HCOs grown in vivo contain progenitor and stem cells.

Global transcriptional analysis of HIOs and HCOs.

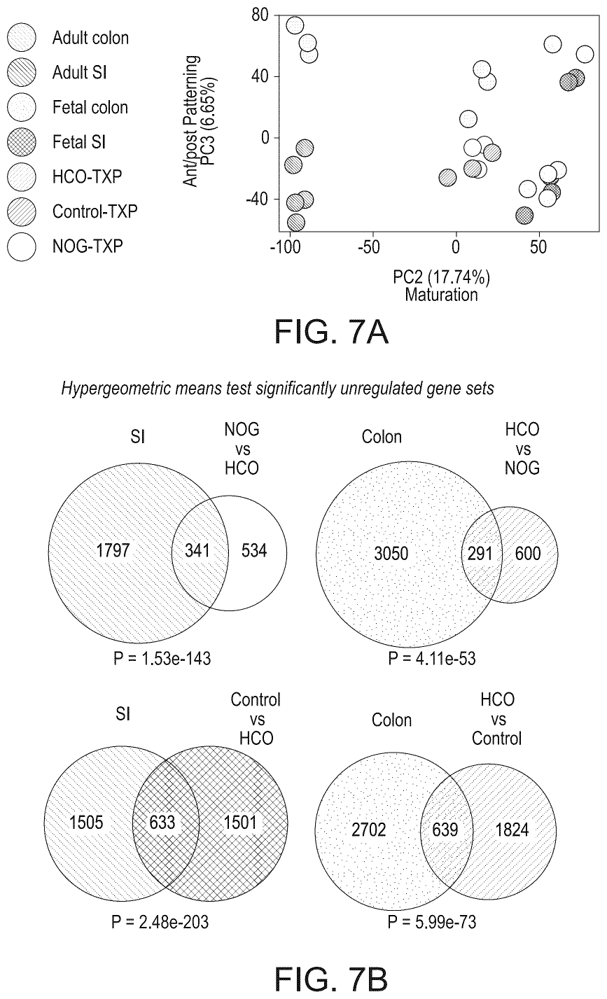

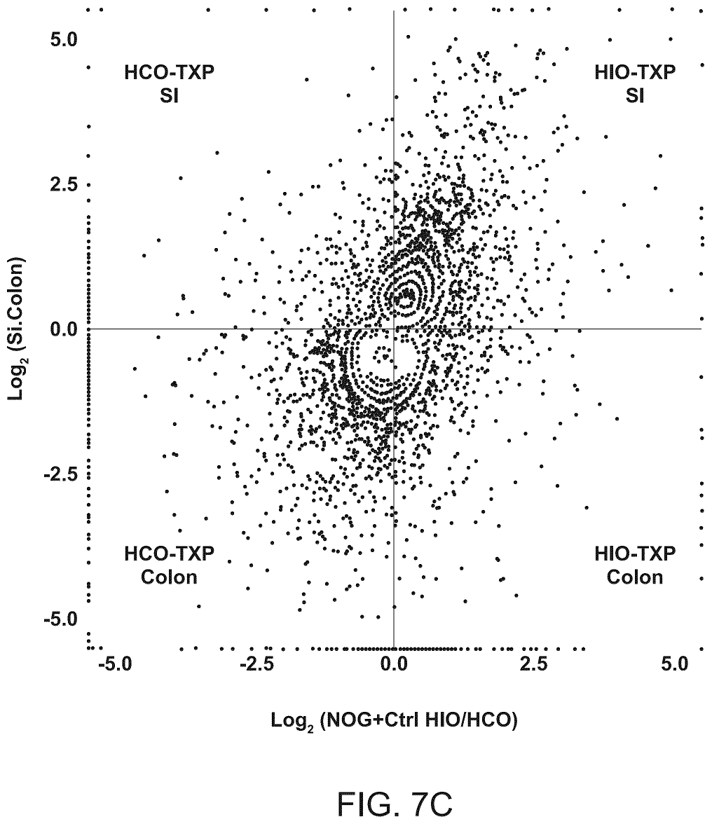

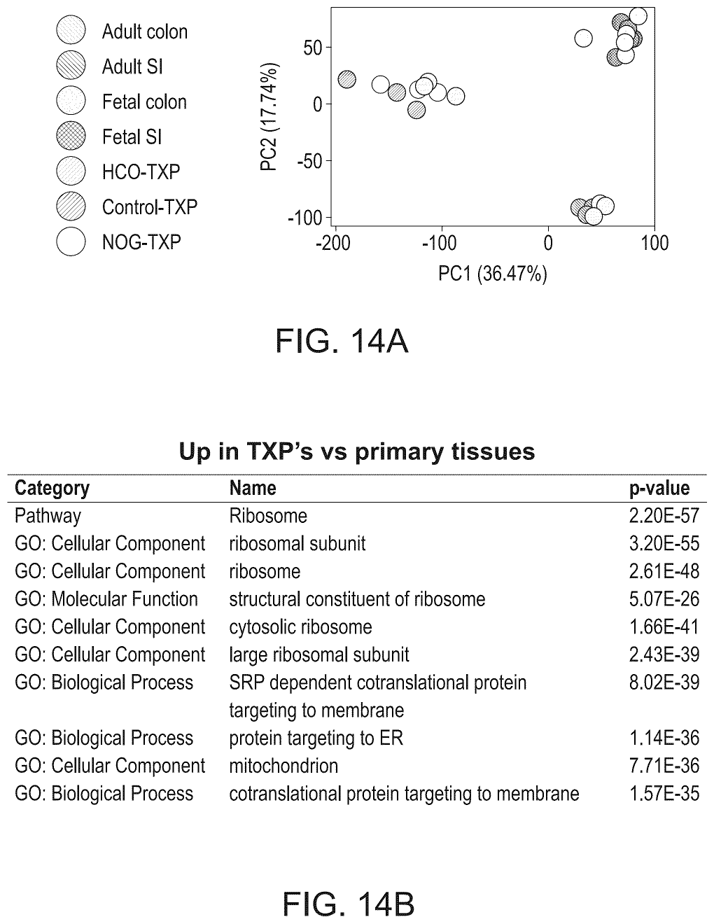

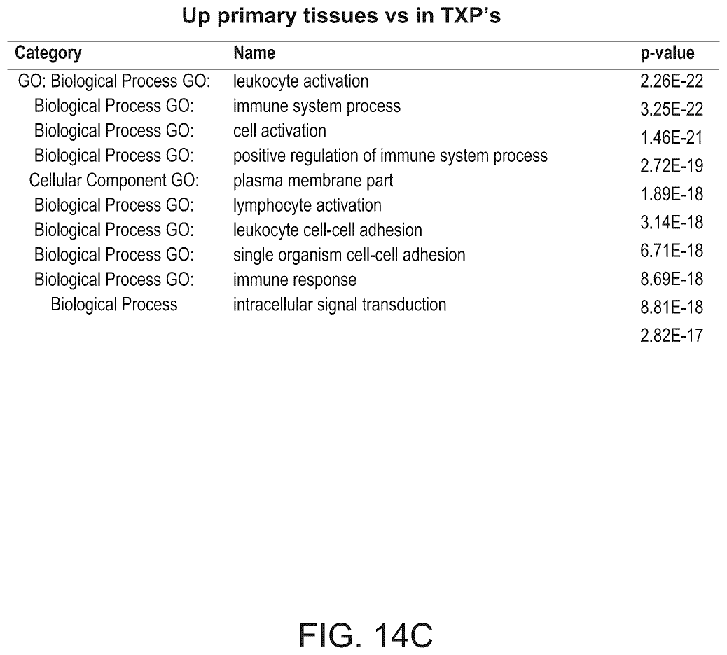

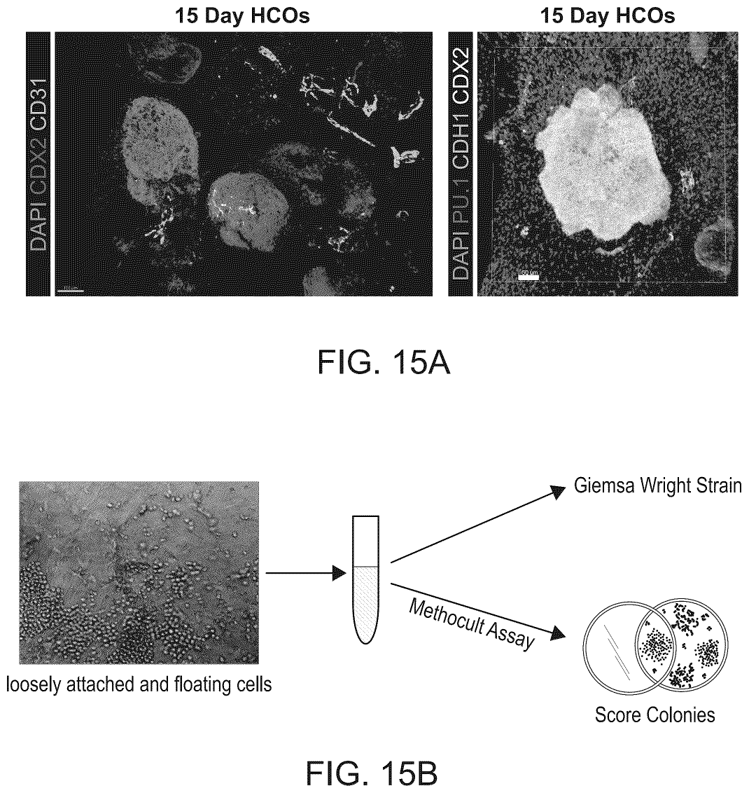

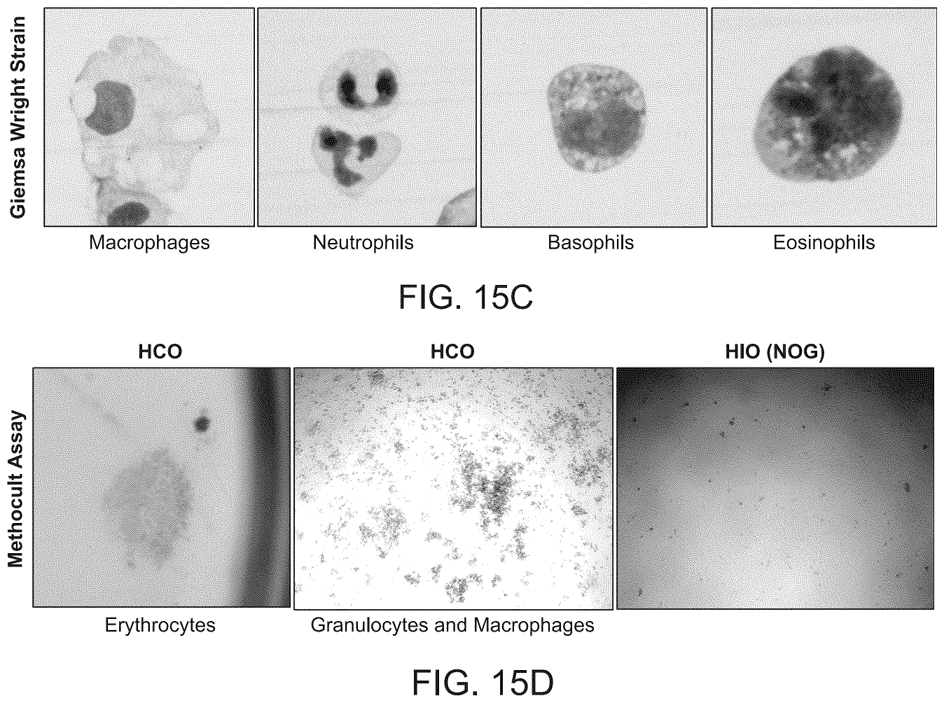

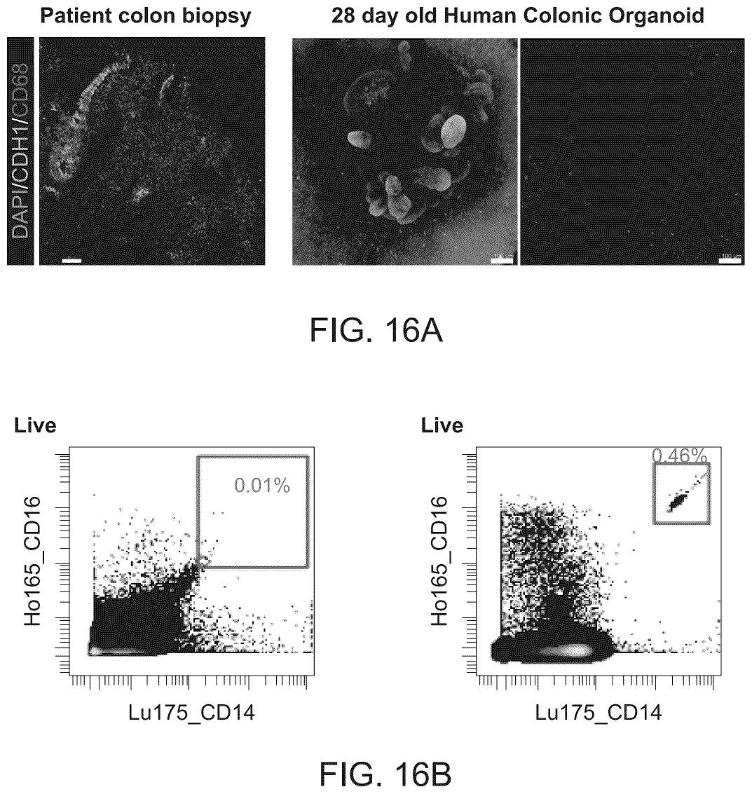

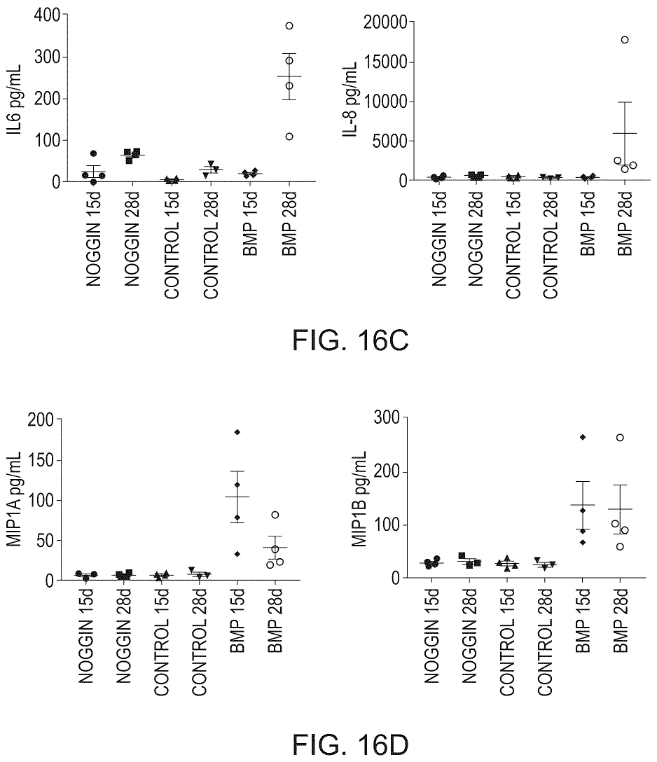

In order to broadly interrogate the regional identity and maturation of HIOs and HCOs, Applicant performed RNA-seq analysis of HIOs and HCOs grown in vivo and compared them with published data sets of human fetal and adult small and large intestines. Principal component analysis revealed that primary tissues isolated from adult and fetal intestine clustered together along principle component 1 (PCI) axis, which accounted for 36.5% of the cumulative variation among samples (FIG. 14A). A GO analysis revealed that this variation was due to cell types that were present only in primary tissues and not PSC-derived transplants. For example, 6 of the top 10 biological processes present in human primary tissues and absent in transplants were related to immune cells (FIG. 14B-C). The second principle component (PC2) accounts for 17.7% of cumulative variation and separates the samples according to maturity (FIG. 7A). This component revealed that transplanted organoids are more mature than human fetal intestine and fetal colon but not as mature as adult colon and intestine. The third principle component (PC3) accounts for 6.7% of cumulative variation and separates the samples according to regional identity, and shows that HCOs are more similar to colon whereas HIOs cluster with small intestine (FIG. 7A). Interestingly, human fetal samples did not cluster based on regional identity (small intestine vs colon) suggesting that these samples may not have been cleanly isolated from the indicated region of the GI tract.

Applicant next used hypergeometric means test to determine the probability that HIOs and HCOs share similar patterns of region-specific gene expression small intestine and colon (FIG. 7B). A total of 341 transcripts are expressed in the small intestine and in NOGGIN treated HIOs as compared to colon or BMP2 treated HCOs, a proportion that is exceedingly unlikely by chance alone (P=1.5×10-143). Similarly, the gene set that is up-regulated in the control HIOs shares a highly significant degree of similarity with the gene set up-regulated in adult small intestine relative to the adult colon (P=2.5×10-203). Conversely, the gene set up-regulated in HCOs are highly enriched for genes that are up-regulated in the colon relative to the small intestine (P=4.1×10-53 and P=6.0×1073, respectively). This analysis concluded that HIO patterning is most similar to human small intestine and HCO patterning is colonic. To further explore the nature of HIOs (NOG and control treated) and HCOs, Applicant conducted differential expression analysis (adult small intestine vs. adult colon; HIOs vs. HCOs). Applicant generated 4-way scatter plot, which also demonstrated that a high proportion of genes up-regulated in the colon were also up-regulated in HCOs and the majority of genes up-regulated in the small intestine were also up-regulated in HIOs (FIG. 7C, Table 1). Lastly, analysis of biological processes that were enriched revealed that adult colon and transplanted HCOs have highly active Wnt signaling and a similar HOX code (FIG. 7D). Taken together, these data suggest Applicant has developed a robust method to differentiate PSCs into human colonic tissue.

Table 1. Genes upregulated in adult small intestine and colon which are also upregulated in HIOs and HCOs respectively. Column 1, Commonly upregulated in NOG HIOs vs HCOs & adult small intestine vs adult colon, Column 2, Commonly upregulated in Control HIOs vs HCOs & adult small intestine vs adult colon, Column 3, Commonly upregulated in HCOs vs NOG HIOs & adult colon vs adult small intestine, Column 4, Commonly upregulated in HCOs vs Control HIOs & adult colon vs adult small intestine

| | | | |
|---|---|---|---|
| ABCA4 | AADAC | AAGAB | AAK1 |
| ABCC2 | ABCA4 | ABCA12 | ABHD10 |
| ABCC6 | ABCB8 | ACTR2 | ABHD17C |
| ABCD1 | ABCC10 | ADAM10 | ABL2 |
| ABCG8 | ABCC6 | ADAM22 | ACTR2 |
| ABHD14A, | | | |
| ABHD14A- | | | |
| ACY1, ACY1 | ABCD1 | AGPS | ACVR2B |
| ABHD6 | ABCG5 | AGTPBP1 | ACYP1 |
| ACBD4 | ABCG8 | AKAP13, MIR7706 | ADAM10 |
| | ABHD14A, | | |
| | ABHD14A-ACY1, | | |
| ACO2 | ACY1 | ALDH1B1 | ADAM17 |
| ACOT11 | ABHD16A | ALG5 | ADAM22 |
| ACP2 | ABHD4 | ANKDD1B | ADGRF4 |
| ACSL5 | ABHD6 | ANKRD30BP2 | AEBP2 |
| ACY3 | ACACB | ANTXR2 | AGPS |
| ADAMTSL5 | ACADS | ANXA3 | AHI1 |
| AGPAT3 | ACADVL | ARFGEF3 | AIDA |
| AKR1B10 | ACKR4 | ARHGAP44 | AIFM3 |
| AKR1C1 | ACO2 | ARPC5 | AKAP11 |
| AKR1C3 | ACOT11 | ASCC1 | AKAP13, MIR7706 |
| ALDOB | ACOT7 | ASIP | AKAP5 |
| ALPI | ACOX2 | ATF6 | ALG8 |
| ANGPTL4 | ACP2 | ATOH1 | ANKDD1B |
| ANXA13 | ACSL5 | ATXN3 | ANKFY1 |
| APOA1 | ACTN4 | B3GALNT2 | ANKS1B |
| APOA1-AS | ACY3 | B3GNT6 | ANP32B |
| APOA4 | ADAMTSL5 | B4GALNT3 | ANTXR2 |
| APOBEC1 | ADGRG4 | BCAS1 | AP2B1 |
| APOBEC2 | ADGRG5 | BCL10 | AP3M1 |
| APOC2, APOC4, | | | |
| APOC4-APOC2 | AGMO | BCLAF1 | APH1B |
| APOC3 | AGPAT1, MIR6721 | BEND3 | APOBR |
| APOL2 | AGPAT2 | BEST2 | ARFGEF3 |
| AQP3 | AGPAT3 | BTBD3 | ARHP1 |
| AQP7 | AK2 | BZW1 | ARL14EP |
| AQP7P1 | AKR1A1 | C11orf58 | ARMC8 |
| ATG4D | AKR1B10 | C12orf75 | ARPC5 |
| AVIL | AKR1C1 | CA12 | ARRDC3 |
| BAK1 | AKR1C3 | CAMSAP1 | ASB7 |
| BCRP3 | AKR1C4 | CAPN2 | ASPH |
| BRE-AS1, RBKS | AKR7A3 | CAPRIN1 | ATG4C |
| BTD | ALDH1A3 | CASC18 | ATL3 |
| BTNL3 | ALDH2 | CBFB | ATM |
| | | CCPG1, DYX1C1, | |
| BTNL8 | ALDOB | DYX1C1-CCPG1 | ATP13A3 |
| C10orf25 | ALPI | CD24 | ATP2A3 |
| C10orf67 | AMBRA1 | CD9 | ATR |
| C11orf24 | ANAPC2 | CDC23 | ATRX |
| C11orf86 | ANGPTL4 | CEACAM6 | ATXN1 |
| C19orf66 | ANPEP | CEP290 | ATXN3 |
| C1orf115 | ANXA13 | CFC1, CFC1B | B3GALNT2 |
| C1orf116 | ANXA4 | CLIC4 | B3GNT6 |
| C5orf56 | AP5B1 | CLMN | BAG5 |
| C6orf132 | APOA1 | CLNS1A | BAZ1B |
| C6orf136 | APOA1-AS | CLSTN1 | BBIP1 |
| C8G | APOA4 | CNTN3 | BCAS1 |
| C9orf173 | APOBEC1 | CSRNP3 | BCAT1 |
| CA13 | APOBEC2 | DAAM1 | BCLAF1 |
| | APOC2, APOC4, | | |
| CALM3 | APOC4-APOC2 | DDX50 | BEND3 |
| CAMKK2 | APOC3 | DGKH | BEST2 |
| CAPN10-AS1 | APOL2 | DICER1 | BIN1 |
| CAPN3 | APOM | DIP2B | BMPR2 |
| CASP1 | AQP3 | DMTN | BNC2 |
| CATSPER2 | AQP7 | EARS2 | BTBD10 |
| CBR1 | ARHGEF16 | EBPL | BTBD3 |
| CCL11 | ASAH2 | EEA1 | BTBD7 |
| CCL25 | ASB13 | EHF | BTF3L4 |
| CD82 | ASIC2 | EPHA10 | BTRC |
| CDCA3 | ASPA | EPHB4 | BZW1 |
| CDH4 | ATG4D | EXOC5 | C10orf99 |
| CDIP1 | ATP11A | EXOC6 | C11orf58 |
| CDR2L | ATP5SL | FAM105A | C18orf54 |
| CERK | ATXN2L | FAM169A | C3orf52 |
| CES2 | AZGP1 | FAM175A | C7orf60 |
| CGREF1 | B3GNT8 | FAM218A | CA12 |
| CHRM4 | BAG6 | FAM60A | CA4 |
| | | | CAMKID, |
| CIB2 | BAIAP2L2 | FAS | LOC283070 |
| CIDEB | BAIAP3 | FECH | CAMSAP2 |
| CIDEC | BAK1 | FERMT1 | CAPN8 |
| CLDN15 | BET1L | FFAR4 | CAPZA1 |
| CMTR1 | BLNK | FGF7 | CASC4 |
| CNNM3 | BRE-AS1, RBKS | FKTN | CASD1 |
| CPS1 | BTD | FMN2 | CBFB |
| CRAT | BTNL3 | FRMPD3 | CBX5, MIR3198-2 |
| CREB3L3 | BTNL8 | G3BP1 | CCDC132 |
| CRIP3 | BUD13 | GARS | CCDC88A |
| CTD-3080P12.3 | C10orf54 | GCSH | CCND1 |
| CYP27A1 | C11orf24 | GGH | CCNJL |
| CYP2B7P | C11orf86 | GJB4 | CCNT1 |
| CYP2C9 | C15orf62 | GLIS3 | CD24 |
| CYP2S1 | C16orf58 | GLUL | CD59 |
| CYP3A5 | C17orf78 | GMNN | CD9 |
| CYTH2 | C18orf8 | GNE | CDC42 |
| DBP | C19orf12 | GNPTAB | CDHR1 |
| DCAF11 | C19orf54 | GOT2 | CDK19 |
| DECR1 | C1orf116 | GP9 | CEACAM5 |
| DGAT1, MIR6848 | C2CD2L | GRSF1 | CEACAM6 |
| DGAT2 | C5orf56 | GSPT1 | CEBPZ |
| DGKA | C6orf132 | GYG2 | CELF2 |
| DHDH | C6orf136 | HDAC1 | CENPO |
| DHRS1 | C8G | HIATL1 | CEP250 |
| DHRS11 | CALCOCO1 | HK2 | CHD9 |
| DHX16 | CALM3 | HMGA2 | CHIC1 |
| DMBT1 | CAMK2G | HN1L | CLIC4 |
| DNAJC22 | CAMTA2 | HNMT | CLMN |
| DNASE1 | CAPN1 | HNRNPAB | CLNS1A |
| DNPEP | CAPNS1 | HOXA10-AS | CLTC |
| | | HOXA10, HOXA10- | |
| DOLPP1 | CARD10 | HOXA9, HOXA9 | CMTM6 |
| DOT1L | CARD6 | HOXA11 | CNEP1R1 |
| DTX1 | CASP1 | HOXB9 | CNTN3 |
| ELMOD3 | CASP4 | HOXB-AS3 | CPM |
| EMB | CASP9 | IARS | CRK |
| ENKUR | CBLC | IFT74-AS1 | CRYM |
| EPHX2 | CBR1 | IL1R2 | CSNK1A1 |
| ERICH4 | CC2D1A | ILDR1 | CSRNP3 |
| ESPN | CCL25 | IPO5P1 | CTDSPL |
| | CD302, LY75, LY75- | | |
| ETV7 | CD302 | IQGAP1 | CTTNBP2NL |
| EXOC3L4 | CD68 | ITM2C | CYLD |
| F10 | CD74 | JAG1 | CYP20A1 |
| FAM102A | CD82 | JPH1 | DAAM1 |
| FAM109A | CDC42BPB | KCNJ2 | DCBLD2 |
| FBP1 | CDC42EP4 | KCNN4 | DCP2 |
| FBXO7 | CDCA3 | KCNRG, TRIM13 | DDX50 |
| FCHSD1 | CDHR5 | KCTD1 | DDX6 |
| FLJ12825 | CDIP1 | KCTD20 | DGKH |
| FLJ22763 | CDK18 | KLK15 | DHRS13 |
| FUOM | CDK2 | KNOP1 | DHRS9 |
| FXR2 | CDK20 | KPNA4 | DHX57 |
| GABRE | CDR2L | LEFTY1 | DICER1 |
| GALK1 | CEACAM18 | LIMK2 | DIO3OS |
| | | | DISC1, TSNAX, |
| GALT | CELA3A | LINC00341 | TSNAX-DISC1 |
| GATA4 | CENPV | LINC00858 | DNAJC3 |
| GATA5 | CERS2 | LIPH | DNAL1 |
| GATS | CES2 | LOC100507346 | DPY19L1 |
| GCHFR | CFI | LOC101928233 | DSEL |
| GIGYF1 | CFL1 | LOC101929395 | DSTYK |
| GNA11 | CGREF1 | LOC101929524 | DTD2 |
| GOLT1A | CHP2 | LRRK1 | EBPL |
| GOSR2 | CHRM4 | MAML2 | EED, MIR6755 |
| GPD1 | CIAO1 | MAPRE2 | EFNA5 |
| GPRIN1 | CIB2 | MARCH3 | EHF |
| GRAMD1B | CIDEB | MARCKSL1 | EI24 |
| GRIA1 | CIDEC | MBNL3 | EID1 |
| GSDMB | CISD1 | MCOLN2 | EIF4E |
| GSK3A | CLDN15 | MECOM | EIF5B |
| GSTK1 | CLDN18 | METTL5 | EMC1 |
| HADHA | CLPTM1 | MFAP3L | ENAH |
| HAGH | CMBL | MFHAS1 | ENDOD1 |
| HAPLN4 | CNNM3 | MINA | ENTPD1 |
| HEBP1 | CNPY2, PAN2 | MLLT3 | EPAS1 |
| HOPX | COASY | MPHOSPH6 | ERCC6L2 |
| HOXA4 | COMMD9 | MPLKIP | ERMP1 |
| HPS1 | COMT, MIR4761 | MPZL2 | ERN2 |
| IDH3A | CPA2 | MREG | EXOC2 |
| IFIT3 | CPPED1 | MRPL1 | EXOC4 |
| IGSF23 | CRADD | MRPL3 | EXOC5 |
| IL2RB | CRAT | MTL5 | EXOC6 |
| INTS12 | CREB3L3 | MYO3A | FAM102B |
| IRF1 | CRELD1 | MYO5C | FAM103A1 |
| IRF8 | CS | NAA15 | FAM105A |
| ISG20 | CSK | NAP1L1 | FAM120A |
| ITPK1 | CTSO | NCBP1 | FAM13B |
| KDM2A | CXCR3 | NDC1 | FAM169A |
| KDM6B | CYB5A | NEDD4L | FAM178A |
| KDM8 | CYP27A1 | NEURL1B | FAM208A |
| KHK | CYP2C19 | NLE1 | FAM46A |
| KIAA2013 | CYP2S1 | NOLC1 | FAM83H-AS1 |
| KIFC3 | CYP3A4 | NOTCH1 | FAM8A1 |
| KLC4 | CYSLTR2 | NOTCH2 | FAM98B |
| LBX2-AS1 | DBP | NRARP | FANCI |
| LINC00574 | DCAF11 | NRXN1 | FAR2P2 |
| LINC01268 | DECR1 | NSF, NSFP1 | FBXO28 |
| LOC100240735 | DEDD | NT5DC3 | FBXO45 |
| LOC284825 | DEGS2 | NUDT4 | FCF1 |
| LOC646471 | DERA | NXPE1 | FEM1B |
| LOC728989 | DESI1 | NXPE2 | FEM1C |
| LPCAT3 | DFNA5 | NXPE4 | FFAR4 |
| LPIN3 | DGAT1, MIR6848 | ODC1 | FKBP5, LOC285847 |
| LRRC75A | DGAT2 | ORC5 | FKTN |
| LRRC75B | DGKA | P4HA1 | FLOT2 |
| MALL | DGKG | PAPPA2 | FNDC3B |
| MAPKBP1 | DGKQ | PARM1 | FOCAD |
| METTL7B | DGKZ | PAWR | FOXD2 |
| MFSD2A | DHDH | PCDHB11 | FOXO3 |
| MGAM | DHRS11 | PDE3B | FOXO3B, ZNF286B |
| MICALL2 | DHRS7 | PDZK1IP1 | FRMPD3 |
| MICU1 | DHX16 | PGBD5 | FRYL |
| MIR1268A, | | | |
| SLC27A4 | DMBT1 | PHF20 | FSIP2 |
| MIR22, MIR22HG | DNAJC22 | PHF6 | FZD4 |
| MIR31HG | DNASE1 | PKIB | G3BP1 |
| MIR3615, | | | |
| SLC9A3R1 | DNPEP | PLCD3 | GGH |
| MIR5187, | | | |
| TOMM40L | DOLPP1 | PLSCR4 | GIN1 |
| MIR5193, UBA7 | DPP9 | POF1B | GJC1 |
| MIR6073, SOX6 | DSCR3 | POLR3B | GLB1L2 |
| MIR621, | | | |
| SLC25A15 | DTX1 | POSTN | GLG1 |
| MIR7703, PSME2 | E2F4 | PP14571 | GLIS3 |
| MISP | EGFR-AS1 | PPIC | GLTSCR1L |
| MME | EIF6 | PPP1R8 | GLUL |
| MMEL1 | ENPP6 | PPP2R3A | GMNN |
| MOCS1 | EPB41L3 | PPP3CA | GNAI1 |
| MOGAT3 | EPHA1 | PREP | GNAQ |
| MON1A | EPHB1 | PRKACB | GNE |
| MS4A8 | EPHX2 | PRKAR2A | GOLGA3 |
| MSRA | EPS8L2 | PRKRIR | GOPC |
| MST1 | EPSTI1 | PRMT5 | GP9 |
| MTTP | ERAL1 | PSMD6 | GPC6 |
| MUS81 | ERF | PSME4 | GPX8 |
| MYO15B | ESPN | PTAR1 | GRM7 |
| MYO1A | ESRRA | PTTG1IP | GRSF1 |
| NAGS | ETV3 | PUM1 | GTF2F2 |
| NELL2 | ETV7 | PYGL | GTF3C1 |
| NGEF | EWSR1 | QPCT | HABP4 |
| NOP9 | EXOC3L4 | R3HDM1 | HEATR3 |
| NPC1L1 | F10 | RAB3B | HIATL1 |
| NR0B2 | FABP2 | RABEP1 | HMG20A |
| NR1I3 | FAH | RAP1GDS1 | HMGA2 |
| | FAM101A, ZNF664, | | |
| NSUN6 | ZNF664-FAM101A | RAPGEF2 | HN1L |
| NUB1 | FAM102A | RBMS3 | HOXA10-AS |
| | | | HOXA10, HOXA10- |
| OGDH | FAM109A | RCC2 | HOXA9, HOXA9 |
| OTC | FAM32A | REXO2 | HOXB5 |
| P4HB | FAM83G | RIF1 | HOXB6 |
| PARP12 | FBLIM1 | RIMS3 | HOXB7 |
| PARP3 | FBP1 | RNASEH2B | HOXB8 |
| PATL2 | FLJ22763 | RPA2 | HOXB9 |
| PCK2 | FUOM | RSF1 | HOXB-AS3 |
| PCSK5 | FZR1 | RSL24D1 | IFFO2 |
| PDLIM2 | GABRA4 | RXFP4 | IFNAR1 |
| PDZD7 | GAL3ST1 | SATB2 | IFT74-AS1 |
| PDZK1 | GALK1 | SATB2-AS1 | IGF1R |
| PEBP1 | GALNT6 | SCLT1 | IGIP |
| PEPD | GALT | SERBP1 | IL17RD |
| PEX14 | GATA4 | SETBP1 | IL1R2 |
| PGRMC2 | GATA5 | SH3PXD2A-AS1 | IL20RB |
| PHEX | GCNT4 | SIPA1L2 | IL6ST |
| PKLR | GFI1B | SLC16A9 | ILDR1 |
| PLA2G6 | GGT1 | SLC1A3 | IMPAD1 |
| PLCB3 | GLOD5 | SLC39A8 | INPP5F |
| | | | IPO11, IPO11- |
| PLEKHS1 | GLRX | SLC7A2 | LRRC70, LRRC70 |
| PLIN2 | GLYCTK | SLC9A2 | IPO5 |
| PLIN3 | GNA11 | SLCO4A1-AS1 | IPO5P1 |
| PLLP | GNB1 | SMAD5 | IPO7 |
| PNP | GOLT1A | SMARCA5 | IQGAP1 |
| PP7080 | GOSR2 | SMC6 | ITGAV |
| PQLC2 | GPD1 | SNRPE | ITGB1 |
| PRAP1 | GPR108 | SNX13 | ITM2A |
| PRDM7 | GPR35 | SOCS5 | ITPRIPL2 |
| PRODH | GRAMD1B | SORBS2 | JAG1 |
| PSMB9 | GRIA1 | SPAG1 | JPH1 |
| PSMD9 | GRK5 | SRSF12 | KBTBD6 |
| PSME1 | GRTP1-AS1 | SRSF9 | KCNJ2 |
| PTPRH | GSDMB | ST6GAL2 | KCNN4 |
| PXDC1 | GSK3A | STAB2 | KCNRG, TRIM13 |
| RAB11FIP3 | GSTA1 | STMND1 | KCTD10 |
| RAB17 | GSTA2 | STS | KCTD20 |
| RAB5C | GSTK1 | STX19 | KDM5B |
| RAB8A | GSTM4 | SUSD1 | KIAA0226L |
| RARRES3 | GTF2I | SUV39H2 | KIAA0232 |
| RBP2 | GTPBP1 | TBL2 | KIAA0513 |
| REEP6 | GUCD1 | TCTA | KIAA1143 |
| REG1A | HADHB | TDGF1 | KIAA1429 |
| RPS6KA1 | HAGH | TFRC | KIAA1715 |
| RTKN | HAPLN4 | TMA16 | KLHL15 |
| RTP4 | HDAC6 | TMCC1-AS1 | KLK15 |
| SAT1 | HDGF | TMED10 | KPNA4 |
| SAT2 | HDHD3 | TMED2 | KRR1 |
| SCAMP5 | HEBP1 | TMEM123 | LAPTM4A |
| SCNN1D | HECTD3 | TMEM159 | LARS |
| SCRN2 | HIP1R | TMEM200B | LEFTY1 |
| SDHA | HLA-F | TMEM38A | LIFR |
| SEC14L2 | HMGA1 | TRABD2A | LIMD1 |
| SERP2 | HNF1A | TSN | LIMK2 |
| SFRP5 | HNF4A-AS1 | TSPAN5 | LINC00341 |
| SFXN3 | HOPX | TTC3 | LINC00482 |
| SH3BP1 | HPS1 | TTC8 | LINC00515 |
| SH3GL1 | HRASLS2 | TTPA | LINC00657 |
| SHBG | HRH2 | UBE2A | LINC01006 |
| SIDT2 | HSD3B7 | UBE2N | LMAN2L |
| SLC12A7 | HYKK | UGP2 | LOC100129550 |
| SLC15A1 | IDNK | UNC13B | LOC100507351 |
| SLC1A7 | IFIT3 | URB1 | LOC101929374 |
| SLC22A4 | IFNLR1 | VWA3B | LOC101929524 |
| SLC23A1 | IGSF23 | WDHD1 | LOC105372441 |
| SLC25A20 | IL22RA1 | WDR35 | LOC731424 |
| SLC25A34 | IL2RB | WDR78 | LOC93622 |
| SLC25A44 | ILVBL | WIPI1 | LRCH2 |
| SLC25A45 | IMMP2L | WNK4 | LRRC37A4P |
| SLC26A11 | INPP5J | WWP1 | LRRC58 |
| SLC2A2 | INTS12 | XPO4 | LUZP6 |
| SLC35B1 | IQSEC2 | YWHAE | LYST |
| SLC37A4 | IRF1 | ZBTB7C | MAFA |
| SLC39A5 | ISG20 | ZBTB8B | MAGEF1 |
| SLC3A2 | ITM2B | ZFHX3 | MAGT1 |
| SLC52A1 | ITPK1 | ZNF658 | MAL2 |
| SLC5A1 | ITPKA | ZNF774 | MAP3K7 |
| SLC5A9 | KALRN | ZNF780B | MARCKS |
| SLC6A19 | KDF1 | | MATR3, SNHG4 |
| SLC6A20 | KDM6B | | MBNL2 |
| SLC7A7 | KDM8 | | MBNL3 |
| SMIM24 | KHK | | MBOAT2 |
| SMIM5 | KIAA0141 | | MBTPS2 |
| SMLR1 | KIAA1551 | | MDN1 |
| SMPD3 | KIAA2013 | | MECOM |
| SOWAHA | KLC4 | | METTL8 |
| SPNS3 | KLHDC8B | | MFAP3L |
| ST7, ST7-OT3 | LASP1 | | MFSD4 |
| STRC | LBX2-AS1 | | MFSD6L |
| SULT1A1 | LEAP2 | | MIB1 |
| SULT1A2 | LHPP | | MIER1 |
| SYK | LINC00319 | | MIOS |
| SYP | LINC00330 | | MIR1244-4, PTMA |
| TAP2 | LINC00483 | | MIR4680, PDCD4 |
| TCF7 | LINC00574 | | MIR6824, SLC26A6 |
| TICAM1 | LINC00667 | | MLF1 |
| TKFC | LINC01137 | | MLLT3 |
| TM4SF20 | LINC01347 | | MLXIP |
| TM4SF4 | LIPE | | MMGT1 |
| TM4SF5 | LIPT1 | | MOB1B |
| TM6SF2 | LMBR1L | | MON2 |
| TMEM150B | LOC100093631 | | MORF4L1 |
| TMEM184A | LOC100506302 | | MPZL1 |
| TMEM253 | LOC100507334 | | MREG |
| TMEM37 | LOC101927051 | | MRPL1 |
| TMEM41A | LOC284825 | | MRPS6, SLC5A3 |
| TMEM82 | LOC90768 | | MTHFD2 |
| TMEM86B | LPCAT3 | | MTMR6 |
| TNFRSF14 | LRP5 | | MTSS1 |
| TNFRSF1A | LRRC28 | | MTURN |
| TNRC6C-AS1 | LRRC41 | | MUC1 |
| TOM1 | LRRC66 | | MUC12 |
| TREH | LRRC75A | | MYO3A |
| TRIM15 | LSMEM2 | | MYO5C |
| TRIM50 | LYRM5 | | NAA15 |
| TTC31 | LZTS3 | | NAA50 |
| TTC38 | MALL | | NAP1L1 |
| | | | NBPF10, NBPF12, |
| | | | NBPF20, NBPF25P, |
| UGT2B7 | MAP2K3 | | NBPF8, NBPF9 |
| UGT3A1 | MAP3K11 | | NCBP1 |
| USH1C | MAPK3 | | NCOA3 |
| WBP2 | MAPKAPK2 | | NDC1 |
| WNT3 | MAPKBP1 | | NEK1 |
| XAF1 | MARC2 | | NEURL1B |
| XDH | MBD1 | | NFIA |
| XPNPEP2 | MCRS1 | | NFYB |
| ZMYND15 | MCUR1 | | NIFK-AS1 |
| ZNF300 | MEP1A | | NKIRAS1 |
| ZSWIM8 | MEP1B | | NOL11 |
| | METTL17 | | NOL9 |
| | METTL7B | | NOTCH1 |
| | MFSD2A | | NPAS1 |
| | MGAM | | NRXN1 |
| | MGAM2 | | NSF, NSFP1 |
| | MGAT3 | | NT5C2 |
| | MGST3 | | NT5DC3 |
| | MICAL1, ZBTB24 | | NUBPL |
| | MICU1 | | NUCKS1 |
| | MIR1268A, | | |
| | SLC27A4 | | NUDT4 |
| | MIR22, MIR22HG | | NUP133 |
| | MIR3615, SLC9A3R1 | | NUP205 |
| | MIR5187, TOMM40L | | NUS1 |
| | MIR5193, UBA7 | | NXPE1 |
| | MIR639, TECR | | NXPE4 |
| | MIR7109, PISD | | OPHN1 |
| | MIR7703, PSME2 | | ORC5 |
| | MISP | | PARM1 |
| | MLF2 | | PBRM1 |
| | MLX | | PCM1 |
| | MLXIPL | | PDE3B |
| | MME | | PDE4D |
| | MMEL1 | | PDS5A |
| | MMP24 | | PDS5B |
| | MOCOS | | PEAK1 |
| | MOCS1 | | PGBD5 |
| | MOGAT2 | | PGGT1B |
| | MOGAT3 | | PGM2L1 |
| | MOGS | | PHC3 |
| | MON1A | | PHF14 |
| | MOV10 | | PHF20 |
| | MPP1 | | PHF6 |
| | MS4A8 | | PHIP |
| | MSRA | | PHTF2 |
| | MST1 | | PIAS2 |
| | MST1R | | PIBF1 |
| | MTTP | | PIGN |
| | MUC17 | | PIGX |
| | MYD88 | | PIK3R3 |
| | MYO15B | | PIKFYVE |
| | MYO19 | | PITHD1 |
| | MYRF | | PJA2 |
| | NAALADL1 | | PKI55 |
| | NAGS | | PKIB |
| | NAPRT | | PKNOX1 |
| | NCK2 | | PLEKHF2 |
| | NCSTN | | PLXNA2 |
| | NELL2 | | POF1B |
| | NGEF | | POLR1E |
| | NIT1 | | POT1 |
| | NLRP6 | | POU2F1 |
| | NOL4L | | PP14571 |
| | NOP9 | | PPIC |
| | NPC1L1 | | PPIP5K2 |
| | NPY6R | | PPM1B |
| | NQO2 | | PPM1K |
| | NR0B2 | | PPP1R3B |
| | NR1H3 | | PPP2R5C |
| | NR1I3 | | PPP3CA |
| | NUCB1 | | PRKACB |
| | NUTM2B-AS1 | | PRKDC |
| | OCIAD2 | | PRKG1 |
| | OGDH | | PRKRIR |
| | OGG1 | | PRPS2 |
| | OTC | | PRRT3-AS1 |
| | OXNAD1 | | PSME4 |
| | P4HB | | PTAR1 |
| | PAOX | | PTEN |
| | PARP2 | | PTGDR |
| | PARP3 | | PTPN14 |
| | PBLD | | PUM1 |
| | PBX2 | | PURB |
| | PCBP2, PCBP2-OT1 | | PWWP2A |
| | PCK2 | | PYGB |
| | PCSK5 | | PYGO1 |
| | PCYT1A | | PYURF |
| | PDE8B | | QPCT |
| | PDLIM2 | | RAB11FIP2 |
| | PDSS1 | | RAB3B |
| | PDXP | | RAB40B |
| | PDZD7 | | RABEP1 |
| | PEBP1 | | RABGAP1 |
| | | | RALGAPA1, |
| | PEPD | | RALGAPA1P |
| | PEX14 | | RANBP2 |
| | PEX16 | | RAP1GDS1 |
| | PFKL | | RAP2A |
| | PFKP | | RAP2B |
| | PGD | | RAPGEF6 |
| | PGRMC2 | | RASA2 |
| | PGS1 | | RASEF |
| | PIM1 | | RBFOX2 |
| | PIP5K1A | | RBM7 |
| | PKLR | | RBMS3 |
| | PLCB3 | | RBPJ |
| | PLEK2 | | RBPMS-AS1 |
| | PLEKHA7 | | RBSN |
| | PLEKHS1 | | RBX1 |
| | PLIN3 | | RCC2 |
| | PLLP | | RDX |
| | PMM1 | | REXO2 |
| | PNLIPRP2 | | RGL3 |
| | PNP | | RIF1 |
| | POLR3H | | RIMKLA |
| | POMGNT1 | | RLIM |
| | POR | | RND3 |
| | PP7080 | | RNF139 |
| | PPIP5K1 | | RNF144A |
| | PPP2R5D | | RNF145 |
| | PPP6R1 | | RNF223 |
| | PQBP1 | | RNMT |
| | PQLC2 | | RSL24D1 |
| | PRAP1 | | RXFP4 |
| | PRDX2 | | SAMD13 |
| | PRKCD | | SAR1A |
| | PRKCZ | | SARAF |
| | PRKD2 | | SATB2 |
| | PRODH | | SATB2-AS1 |
| | PRR13 | | SBNO1 |
| | PRSS1 | | SCAI |
| | PRSS3P2 | | SCFD1 |
| | PSD4 | | SDC4 |
| | PSMA1 | | SEC22A |
| | PSMB10 | | SEC23IP |
| | PSMB8 | | SEC62 |
| | PSMB9 | | SECISBP2L |
| | PSMD9 | | SEL1L |
| | PSME1 | | SEMA3C |
| | PTK2B | | SEMA3D |
| | PTPRH | | SEMA5A |
| | PVRL2 | | SEPT11 |
| | PXDC1 | | SEPT7 |
| | QRICH1 | | SERBP1 |
| | RAB11FIP3 | | SERINC5 |
| | RAB17 | | SERTAD2 |
| | RAB5C | | SESN1 |
| | RAB8A | | SESN3 |
| | RARA | | SETX |
| | RARRES3 | | SH3PXD2A-AS1 |
| | RASSF4 | | SHOC2 |
| | RBP2 | | SHROOM4 |
| | REEP6 | | SIPA1L2 |
| | REGIA | | SLC10A7 |
| | RFX5 | | SLC15A2 |
| | RGN | | SLC16A9 |
| | RIC3 | | SLC19A2 |
| | RIPK3 | | SLC1A3 |
| | RIPK4 | | SLC22A15 |
| | RMDN3 | | SLC25A12 |
| | RMND1 | | SLC25A30 |
| | RNF10 | | SLC2A10 |
| | RNF123 | | SLC30A6 |
| | RNF167 | | SLC35A1 |
| | RRNAD1 | | SLC37A3 |
| | RTKN | | SLC38A2 |
| | RTP4 | | SLC38A6 |
| | SAPCD1- | | |
| | AS1, VWA7 | | SLC39A8 |
| | SAT2 | | SLC44A1 |
| | SCAMP5 | | SLC7A2 |
| | SCARB1 | | SLC9A2 |
| | SCML4 | | SMAD5 |
| | SCNN1D | | SMARCA5 |
| | SCRN2 | | SMC6 |
| | SDHD | | SMG1 |
| | SEC13 | | SMIM14 |
| | SEC14L2 | | SNRPE |
| | SEC16B | | SNX13 |
| | SFRP5 | | SOCS5 |
| | SFXN3 | | SORBS2 |
| | SH3BP1 | | SPAG1 |
| | SH3GL1 | | SPIN2B |
| | SHPK, TRPV1 | | SPIRE1 |
| | SIDT2 | | SPTAN1 |
| | SIGLEC12 | | SPTSSA |
| | SLC12A7 | | SRSF12 |
| | SLC13A2 | | SSB |
| | SLC16A13 | | SSR1 |
| | SLC16A5 | | SSR3 |
| | SLC19A1 | | ST6GAL2 |
| | SLC22A18 | | ST7L |
| | SLC22A4 | | STAB2 |
| | SLC23A1 | | STAG1 |
| | SLC25A20 | | STMND1 |
| | SLC25A44 | | STRN3 |
| | SLC25A45 | | STS |
| | SLC25A5 | | STX19 |
| | SLC26A11 | | STX6 |
| | SLC2A12 | | SUMF1 |
| | SLC2A5 | | SUPT16H |
| | SLC2A9 | | SUV39H2 |
| | SLC35B1 | | SYT7 |
| | SLC37A4 | | SYTL2 |
| | SLC39A5 | | SYTL4 |
| | SLC3A2 | | TACC1 |
| | SLC52A1 | | TAF9B |
| | SLC5A1 | | TAOK1 |
| | SLC5A6 | | TBL1X |
| | SLC6A19 | | TCAM1P |
| | SLC6A20 | | TDGF1 |
| | SLC7A7 | | TEAD1 |
| | SLC7A9 | | TFCP2L1 |
| | SLC9A3 | | TFRC |
| | SLX4 | | TGFBR1 |
| | SMAD3 | | THADA |
| | | | TICAM2, TMED7, |
| | SMARCD1 | | TMED7-TICAM2 |
| | SMIM24 | | TINCR |
| | SMLR1 | | TLK1 |
| | SMOX | | TLN2 |
| | SMPD3 | | TMCC1-AS1 |
| | SOAT2 | | TMED10 |
| | SPANXN3 | | TMED9 |
| | SPHK2 | | TMEM106B |
| | SPNS3 | | TMEM123 |
| | SRC | | TMEM159 |
| | SSTR1 | | TMEM194A |
| | ST7, ST7-OT3 | | TMEM194B |
| | STAT6 | | TMEM2 |
| | STAU1 | | TMEM44 |
| | STK24 | | TMEM45A |
| | SUCLG1 | | TMEM87A |
| | SULT1A1 | | TMX1 |
| | SULT1A2 | | TNFRSF10D |
| | SULT2A1 | | TNKS |
| | SYP | | TNRC6C |
| | TAP2 | | TOP2B |
| | TBK1 | | TP53BP1 |
| | TCF7 | | TP53INP1 |
| | TFG | | TRABD2A |
| | THRA | | TRIM23 |
| | TIAM2 | | TRIM37 |
| | TICAM1 | | TRIP12 |
| | TJAP1 | | TRMT5 |
| | TKFC | | TROVE2 |
| | TLDC2 | | TSN |
| | TM4SF4 | | TSPAN5 |
| | TM4SF5 | | TSPAN6 |
| | TM6SF2 | | TSPYL4 |
| | TMED4 | | TTC28 |
| | TMEM116 | | TTC3 |
| | TMEM120A | | TTL |
| | TMEM139 | | TTPA |
| | TMEM150B | | TTPAL |
| | TMEM177 | | TWISTNB |
| | TMEM184A | | TWSG1 |
| | TMEM229B | | TXNDC15 |
| | TMEM253 | | UBE2D1 |
| | TMEM25, TTC36 | | UBE2Q2 |
| | TMEM37 | | UBR5 |
| | TMEM51 | | UGGT1 |
| | TMEM51-AS1 | | UGGT2 |
| | TMEM82 | | UNC5C |
| | TMEM86B | | USP13 |
| | TMEM92 | | USP24 |
| | TNFRSF14 | | USP34 |
| | TNFRSF1A | | USP47 |
| | TNIK | | USP53 |
| | TOM1 | | UXS1 |
| | TOM1L1 | | VANGL1 |
| | TOX4 | | VKORC1L1 |
| | TPI1 | | VOPP1 |
| | TRAF4 | | VPS13B |
| | TREH | | VPS13C |
| | TRIM14 | | WAC-AS1 |
| | TRIM15 | | WBP5 |
| | TRIM16 | | WDFY1 |
| | TRIM21 | | WDHD1 |
| | TTC38 | | WDR36 |
| | UBXN2A | | WDR7 |
| | UGT3A1 | | WDR78 |
| | UNC5CL | | WDR89 |
| | USF1 | | WNK4 |
| | USH1C | | XIAP |
| | USP10 | | XPNPEP3 |
| | USP2 | | XPO4 |
| | VRK3 | | YTHDF3 |
| | VRTN | | ZBTB10 |
| | WBP2 | | ZBTB7C |
| | WDR45 | | ZDHHC7 |
| | WDTC1 | | ZFHX3 |
| | WNT3 | | ZFP90 |
| | WWC1 | | ZFX |
| | XAF1 | | ZMAT2 |
| | XDH | | ZMPSTE24 |
| | XPNPEP1 | | ZMYM4 |
| | ZDHHC9 | | ZNF148 |
| | ZER1 | | ZNF260 |
| | ZFAND3 | | ZNF264 |
| | ZFYVE27 | | ZNF320 |
| | ZMYND15 | | ZNF555 |
| | ZNF384 | | ZNF644 |
| | ZNF782 | | ZNF652 |
| | ZRANB2-AS1 | | ZNF678 |
| | ZSWIM8 | | ZNF69 |
| | | | ZNF704 |
| | | | ZNF709 |
| | | | ZNF766 |
| | | | ZNF780B |
| | | | ZNF81 |
| | | | ZYG11B |

### DISCUSSION

Historically, the classification of foregut, midgut, and hindgut are based on the development of the anterior and posterior intestinal portals and the source of mesenteric blood supply (Uppal et al., 2011). An alternative definition of midgut and hindgut have been proposed, in which the midgut is the portion of the intestine derived from the portion anterior to the umbilicus and the hindgut derives posterior to the umbilicus (Johnston, 1913; Savin et al., 2011). In either case, the historic reliance on anatomical landmarks, and lack of more precise molecular markers to distinguish fore, mid and hindgut, have made it difficult to develop methods to generate these cell/tissues in vitro from PSCs. Therefore, identification of markers that clearly demarcate regions of developing mid and hindgut is essential.

Applicant used a combination of CDX2, GATA4, ONECUT1 and SATB2 to identify that distinct molecular boundaries are established at early stages of mid and hindgut development in Xenopus, mouse and humans. Interestingly, GATA4 and SATB2 expression domains form a boundary at the yolk stalk/presumptive umbilical cord in mice, and this boundary is maintained throughout development and in the adult intestine. The fact that GATA4 expression marks the intestine anterior to the umbilicus, and SATB2 expression marks the domain posterior to the umbilicus, suggests that the umbilicus is the boundary between the midgut and hindgut (Johnston, 1913; Savin et al., 2011).

While ONECUT1 expression in HIOs and SATB2 expression is HCOs is consistent with their proximal and distal identify respectively, GATA4 was not as robustly expressed in proximal HIOs in vitro as would be expected given its embryonic expression (data not shown). In contrast, GATA4 was robustly expressed following in vivo maturation of HIOs and in enteroids generated from patient biopsies (data not shown). This could suggest that factors involved in expression of GATA4 are absent in culture conditions or that maturation in vivo is required for epithelial expression of GATA4. This data also suggests that high levels of GATA4 expression may be dispensable for early regionalization of the intestine, consistent with intestinal Gata4 knockout mice that retain normal Onecut factor expression (Battle et al., 2008). In addition, a small subset of BMP treated organoids lost CDX2 expression and activated expression of the bladder markers Keratin 13 and Uroplakin 1a (data not shown). This is consistent with BMP organoids having a hindgut fate since urothelial tissue is derived from the hindgut/cloaca (Georgas et al., 2015).

SATB2 is expressed throughout development of the distal ileum and large intestine, however it is not known if SATB2 is required for development of the distal intestine. Mouse knockout studies have focused on craniofacial and cortical neuronal development since mutations in SATB2 has been implicated in Cleft Palate associated with 2q32-q33 deletions and Glass Syndrome (FitzPatrick et al., 2003). However, there is indirect evidence that SATB2 may play a role human colonic physiology. SATB2 has been identified in Genome Wide Association Studies as an ulcerative colitis susceptibility gene (McGovern et al., 2010). In addition, loss of SATB2 expression has been shown to be associated with poor prognosis in colorectal cancer patients (Eberhard et al., 2012). Future studies with HCOs may allow identification of SATB2 targets in the developing colon, which could provide insight into the pathology of ulcerative colitis and colorectal cancer.

Several studies in model organisms have implicated the BMP signaling pathway in patterning endoderm during hindgut development (Kumar et al., 2003; Roberts et al., 1995; Sherwood et al., 2011; Tiso et al., 2002; Wills et al., 2008). Consistent with this, Applicant has demonstrated that posterior patterning of human definitive endoderm is dependent on BMP signaling, as inhibition of BMP abrogates the ability of WNT and FGF to promote a posterior endoderm fate (McCracken et al., 2014). However, it is not surprising that BMP signaling plays other temporally distinct roles during intestinal development. For example, after the establishment of proximal-distal regional domains, BMP signaling functions to establish the crypt-villus axis in the intestine and colon (Li, 2005). Thus, a temporal requirement for patterning allows the embryo to use the same signaling pathway for multiple purposes gut development, as has been reported in Drosophila midgut (Driver and Ohlstein, 2014; Guo et al., 2013). In a human disease context, mutations in BMPRIA are associated with a subset of patients with Juvenile Polyposis Syndrome. The HCO system was highly amenable for identifying the HOX code that is downstream of BMP during early development and it could be interesting to determine if hamartomatous polyps with BMPR1A mutations have altered HOX gene expression.

Applicant previously reported the in vitro directed differentiation and in vivo transplantation of HIOs (Spence et al., 2011; Watson et al., 2014), which were small intestinal. Given the unique physiology and pathological conditions that affect the large intestine, it was imperative to develop a colonic model system to interrogate pathophysiological questions specific to the colon. Developmentally, this system provides the opportunity to investigate fundamental questions about how regional identity is established. HIOs and HCOs develop unique cell types, such as Paneth cells in the HIOs and colon-specific goblet cells in HCOs. Moreover, HIOs and HCOs have a distinct set of EECs that are normally enriched in the small and large intestine, respectively. Regionalized organoids should provide a platform for future studies of how different regions of the intestine give rise to regionalized stem cells. In addition, generation of HCOs will allow for modeling of diseases that affect the colon such as ulcerative colitis and colorectal cancer.

### Materials and Methods

Animals. Immune-deficient NOD-SCID IL-2Rynu″ (NSG) mice, 8-16 weeks old, were used in transplantation experiments (obtained from the Comprehensive Mouse and Cancer Core Facility, Cincinnati, Ohio). Wild type mice were used for studies on mouse fetal intestine. All mice were housed in the animal facility at the Cincinnati Children's Hospital Medical Center (CCHMC). All experiments were performed with the approval of the Institutional Animal Care and Use Committee of CCHMC.

BMP inhibition in frog and mouse embryos. Xenopus tropicalis embryo culture and small molecule treatments were performed as previously described (Rankin et al., 2012; Rankin et al., 2015). DMH-1 (Sigma D8946) was dissolved in DMSO, and used at final concentration of 20 μM; equal concentrations of DMSO vehicle were used on sibling embryos. Inhibitor treatment experiments were repeated twice with similar effects on the markers analyzed. For Xenopus in-situ hybridization analyses, DIG-labeled antisense RNA probes were generated using linearized full-length cDNA plasmid templates (X. tropicalis satb2 was purchased from ATCC, clone 7720194; HinDIII, T7 for probe; X. laevis satb2 was a gift for Tyler Square and Daniel Medeiros, University of Colorado-Boulder; Xbal, Sp6 for probe). Complete details describing probe synthesis and the in-situ hybridization protocol are available on Xenbase (http://wiki.xenbase.orq/xenwiki/index.php/Protocols).

For mouse whole embryo cultures, e7.5 embryos were cultured in a 1:1 mixture of Ham's F12 medium and whole embryo culture rat serum (Harlan Labs) containing N-2 Supplement (Invitrogen). Vessels were placed on a roller culture apparatus (BTC Engineering, Cambridge, UK) and maintained for 2 days at 37° C. and gassed with 20% 02 and 5% C02. BMP signaling was inhibited by treatment with 5 μM DMH-1, with DMSO serving as a vehicle control.

Generation of human midgut/hindgut spheroids. Human intestinal organoids were generated and maintained as previously described (Watson et al., 2014). Human embryonic stem cells and induced pluripotent stem cells were grown in feeder-free conditions in six-well Nunclon surface plates (Nunc) coated with Matrigel (basement membrane matrix, BD Biosciences) and maintained in mTESR1 media (Stem Cell Technologies). For induction of definitive endoderm (DE), human ES or iPS cells were passaged with Accutase (Invitrogen) and plated at a density of 100,000 cells per well in a Matrigel-coated, Nunclon surface 24-well plate. For Accutase split cells, 10 μM Y27632 compound (Sigma) was added to the media for the first day. After the first day, media was changed to mTESR1 and cells were grown for an additional 24 hours. Cells were then treated with 100 ng/niL of Activin A for 3 days as previously described (Spence et al., 2011). DE was then treated with hindgut induction medium (RPMI 1640, 2 mM L-glutamine, 2% decomplemented FBS, penicillin-streptomycin and 100 ng/mL Activin A) for 4 d with 500 ng/mL FGF4 (R&D) and 3 μM Chiron 99021 (Tocris) to induce formation of mid-hindgut spheroids.

Patterning midgut/hingut spheroids into HIOs and HCOs. Spheroids were collected from 24 well plate and plated in Matrigel (BD). To generate proximal HIOs, spheroids were overlay ed with intestinal growth medium (Advanced DMEM/F-12, N2, B27, 15 mM HEPES, 2 mM L-glutamine, penicillin-streptomycin) supplemented with 100 ng/mL EGF (R&D) alone, or 100 ng/mL EGF with 100 ng/ml NOGGIN (R&D). To generate HCOs, spheroids were overlayed with 100 ng/mL EGF plus 100 ng/mL BMP (R&D). For SHH experiments, 1 μM SAG (Tocris), 5 μM SAG or 2.5 μM Cyclopamine (Tocris) were added to control media for initial 3 days after which RNA samples were collected. Media was changed at 3 days with only EGF being maintained in the media for all patterning conditions. Media was then changed twice weekly thereafter. HIOs and HCOs were replated in fresh Matrigel every 14 days.

Generation of NEUROGENIN3 inducible line. To generate a doxycycline inducible NEUROG3 line, Applicant transduced IPSC 72.3 cells with pINDUCER21-NEUROG3 lentivirus and selected using 250 g/mL of G418. Both the IPSC 72.3 cell line and the inducible NEUROG3 have been described previously (McCracken et al., 2014). Stably transduced cells were differentiated into mid/hindgut spheroids and then patterned into HIOs or HCOs. Spheroids were grown for 28 days and were pulsed with 0.5 ug/mL of doxycycline for 8 hrs. At day 35, organoids were collected and were analyzed by QPCR and IF.

Growth of organoid mesenchyme. Mesenchymal cells from organoids which attach to the bottom of the 24-well plate attach and grow in 2 dimensions. To expand mesenchymal cells from organoids, DMEM 10% FBS+L-glutamine+penicillin-streptomycin was added to wells from which organoids had been harvested at 14 days. Media was changed twice weekly for a total of 2-3 weeks until near 100% confluence was achieved.

Transplantation of human intestinal organoids. NSG mice were kept on antibiotic chow (275 p.p.m. Sulfamethoxazole and 1,365 p.p.m. Trimethoprim; Test Diet). Food and water was provided ad libitum before and after surgeries. A single HIO, matured in vitro for 28 days, was removed from Matrigel, washed with cold phosphate-buffered saline (DPBS; Gibco), and embedded into purified type I collagen (rat tail collagen; BD Biosciences) 12 hours before surgery to allow for formation of a solidified gel plug. These plugs were then placed into standard growth media overnight in intestinal growth medium (Advanced DMEM/F-12, B27, 15 mM HEPES, 2 mM L-glutamine, penicillin-streptomycin) supplemented with 100 ng/niL EGF (R&D). HIOs were then transplanted under the kidney capsule as previously reported (Watson et al., 2014). Briefly, the mice were anesthetized with 2% inhaled isoflurane (Butler Schein), and the left side of the mouse was then prepped in sterile fashion with isopropyl alcohol and povidine-iodine. A small left-posterior subcostal incision was made to expose the kidney. A subcapsular pocket was created and the collagen-embedded HIO was then placed into the pocket. The kidney was then returned to the peritoneal cavity and the mice were given an IP flush of Zosyn (100 mg/kg; Pfizer Inc.). The skin was closed in a double layer and the mice were given a subcutaneous injection with Buprenex (0.05 mg/kg; Midwest Veterinary Supply). At 8-10 weeks following engraftment, the mice were then humanely euthanized or subjected to further experimentation.

Tissue processing, immunofluorescence and microscopy. Tissues were fixed for 1-3 hours in 4% paraformaldehyde (PFA) on ice depending on the size of the tissue. Organoids and transplant engraftments were frozen in OCT. OCT sections were blocked using donkey serum (5% serum in IX PBS plus 0.5% Triton-X) for 30 min and incubated with primary antibody overnight at 4° C. Slides were then washed 3× with IX PBS plus 0.5% Triton-X and incubated in secondary antibody with DAPI in blocking buffer for 2 h at room temperature. See Table 2 for a list of antibodies and respective dilutions. Slides were then washed 2× with IX PBS plus 0.5% Triton-X followed by a final wash in IX PBS. Coverslips were then mounted using Fluoromount-G® (SouthernBiotech). Images were captured on a Nikon A1 confocal microscope and analyzed using Imaris Imaging Software (Bitplane). For whole-mount staining, tissues were processed similarly as above and then cleared in Murray's solution. Imaging was performed with a Nikon A1 confocal microscope.

TABLE 2
QPCR primers used. See FIGS. 3 and 4.

| GENE | Sequence |
|---|---|
| CDH1 FWD | GACCGGTGCAATCTTCAAA |
| CDH1 REV | TTGACGCCGAGAGCTACAC |
| CHGA FWD | TGTGTCGGAGATGACCTCAA |
| CHGA REV | GTCCTGGCTCTTCTGCTCTG |
| CKB FWD | CCCACACCAGGAAGGTCTTA |
| CKB REV | CCTCTTCGACAAGCCCGT |
| FXYD3 FWD | AGGGTCACCTTCTGCATGTC |
| FXYD3 REV | CTTCGGATAAACGCAGGACT |
| GATA4 FWD | TAGCCCCACAGTTGACACAC |
| GATA4 REV | GTCCTGCACAGCCTGCC |
| HOXA13 FWD | GCACCTTGGTATAAGGCACG |
| HOXA13 REV | CCTCTGGAAGTCCACTCTGC |
| HOXB13 FWD | GCTGTACGGAATGCGTTTCT |
| HOXB13 REV | AACCCACCAGGTCCCTTTT |
| HOXD13 FWD | CCTCTTCGGTAGACGCACAT |
| HOXD13 REV | CAGGTGTACTGCACCAAGGA |
| HOXD3 FWD | CACCTCCAATGTCTGCTGAA |
| HOXD3 REV | CAAAATTCAAGAAAACACACACA |
| INSL5 FWD | GAAGGTTTTGCGCTGGATT |
| INSL5 REV | GATCCCTCAAGCTCAGCAAG |
| MSX2 FWD | GGTCTTGTGTTTCCTCAGGG |
| MSX2 REV | AAATTCAGAAGATGGAGCGG |
| MUC2 FWD | TGTAGGCATCGCTCTTCTCA |
| MUC2 REV | GACACCATCTACCTCACCCG |
| ONECUT1 Fwd | TTTTTGGGTGTGTTGCCTCT |
| ONECUT1 Rev | AGACCTTCCGGAGGATGTG |
| PDX1 FWD | CGTCCGCTTGTTCTCCTC |
| PDX1 REV | CCTTTCCCATGGATGAAGTC |
| PPIA (CPHA) FWD | CCCACCGTGTTCTTCGACATT |
| PPIA (CPHA) REV | GGACCCGTATGCTTTAGGATGA |
| SATB2 FWD | CCACCTTCCCAGCTTGATT |
| SATB2 REV | TTAGCCAGCTGGTGGAGACT |

Quantification of immunofluorescence images. Image quantitation of whole embryos was done by splitting images into separated channels and then measuring pixel area using ImageJ (NIH). Pixel area was determined for each channel, the ratio between channels was determined and the ratio for control treated embryos was represented as 100. Quantitation of in vitro and in vivo grown organoids was done on sections from which images were captured as explained above. The number of CDX2, GATA4 and SATB32 positive nuclei were quantified using the spot function in [marls following calibration with human biopsy samples.

RNA isolation and QPCR. RNA was extracted using Nucleospin® RNA extraction kit (Macharey-Nagel) and reverse transcribed into cDNA using Superscript VILO (Invitrogen) according to manufacturer's protocols. QPCR primers were designed using the qPrimerDepot webased tool (primerdepot.nci.nih.gov). Primer sequences are listed in Table 3. QPCR was performed using Quantitect SYBR® Green PCR kit (Qiagen) and a QuantStudio TM 6 Flex Real-Time PCR System (Applied Biosystems).

TABLE 3
Antibodies used. See FIGS. 1-6.

| ANTIBODY | HOST | Catalog number | Dilution |
|---|---|---|---|
| B-Catenin | rabbit | Santa Cruz #sc-7199 | 1:200 |
| CDH17* | rabbit | Sigma #HPA023616 | 1:1,500 |
| Cdx2 | mouse | BioGenex cdx2-88 | 1:300 |
| Cdx2 | rabbit | Cell Marque EPR2764Y | 1:100 |
| | monoclonal | | |
| Chr-A (C20) | goat | Santa Cruz #sc-1488 | 1:100 |
| DEFA5* | mouse | Novus | 1:60,000 |
| | monoclonal | BiologicalsNB110- | |
| | | 60002 | |
| E-Cadherin | goat | R&D #AF648 | 1:400 |
| E-Cadherin | rat | R&D #MAB7481 | 1:500 |
| (mouse-specific) | | | |
| E-Cadherin | mouse | R&D #AF648 | 1:500 |
| FoxA2 | goat | Santa Cruz #sc-6554 | 1:500 |
| GATA4 | goat | Santa Cruz #sc-1237 | 1:100 |
| GATA4 | rabbit | Santa Cruz #sc-9053 | 1:100 |
| GFP (green | rabbit | Invitrogen #A11122 | 1:1,000 |
| fluorescent protein) | | | |
| Ghrelin | goat | Santa Cruz #sc-10368 | 1:500 |
| GIP (Gastric | goat | Santa Cruz #sc-23554 | 1:500 |
| Inhibitory | | | |
| Polypeptide) | | | |
| GLP-1 | mouse | BioVision #3104-100 | 1:200 |
| HNF-6 (ONECUT1) | rabbit | Santa Cruz #sc-13050 | 1:100 |
| INSL5 (H-110)* | rabbit | Santa Curz #sc-67190 | 1:100 |
| KI67 | rabbit | Cell Marque SP6 | 1:100 |
| | monoclonal | | |
| Motilin | mouse | Santa Cruz #sc-376605 | 1:100 |
| | monoclonal | | |
| Mucin 5B* | rabbit | Santa Cruz #sc-20119 | 1:100 |
| Mucin2 (MUC2) | rabbit | Santa Cruz #sc-15334 | 1:200 |
| Peptide YY | rabbit | Abcam #ab22663 | 1:1000 |
| pSmad 1/5/8 | rabbit | Cell Signaling 9511S | 1:100 |
| (Discontinued | | | |
| and replaced | | | |
| with 13820S) | | | |
| pSmad 2/3 | rabbit | Cell Signaling 9510S | 1:100 |
| SATB2 | rabbit | Cell Marque EP281 | 1:100 |
| | monoclonal | | |
| SATB2 | mouse | Santa Cruz #sc-81376 | 1:100 |
| (SATBA4610)* | monoclonal | | |
| Sox9 | rabbit | Millipore #AB5535 | 1:10,000 |
| Alexafluor ® | donkey | Life Technologies A- | 1:500 |
| Donkey anti- | | 11055 | |
| goat 488 | | | |
| Alexafluor ® | donkey | Life Technologies A- | 1:500 |
| Donkey anti- | | 11057 | |
| goat 568 | | | |
| Alexafluor ® | donkey | Life Technologies A- | 1:500 |
| Donkey anti- | | 10037 | |
| mouse 568 | | | |
| Alexafluor ® | donkey | Life Technologies A- | 1:500 |
| Donkey anti- | | 31573 | |
| rabbit 647 | | | |
| Alexafluor ® | donkey | Life Technologies A- | 1:500 |
| Donkey anti- | | 21208 | |
| rat 488 | | | |

Identification of SAT132 as a large intestinal marker.

To identify markers of large intestine, Applicant first used GNCPro http://gncpro.sabiosciences.comigncpro/expression_grapherphp to identify transcription factors upregulated in colon (compared to other tissues) based on the University of Tokyo database. Based on this search, SATB2 was the 6th ranked gene in colon. To verify that SATB2 is indeed upregulated in the colon, Applicant searched SATB2 expression using the TiGER database (hftp://bioinfo.wilmer.ihu.edu/tiger/db gene/SATB2-index.html). To further confirm the expression of SATB2 in the colon, and to examine protein expression across numerous tissues, Applicant used the Human Protein Atlas (http://www.proteinatlas.org/search/satb2). A similar approach was used to identify other markers of large intestine/colon.

Public RNA-seq accession numbers. Adult small intestine and large intestine RNA-seq data were downloaded from the public database E-MTAB-1733. These data sets represent whole organ tissue which includes the epithelium and muscle layers. Accession numbers for the small intestine samples: ERR315344, ERR315381, ERR315409, ERR315442, ERR315461. Accession numbers for the large intestine samples: ERR315348, ERR315357, ERR315484. For FIG. 9B, processed FPKM data was downloaded from https://qithub.com/hilldr/Finkbeiner StemCellReports2015. These data include adult duodenum (ERS326992, ERS326976) and small intestine samples listed above from E-MTAB-1733 as well as human fetal intestinal (also whole organ) samples from GSE18927. Accession numbers for human fetal small intestine are GSM1059508, GSM1059521, GSM1059486, GSM1059507, GSM1059517, GSM1220519. For FIG. 9C, data was obtained from GEO accession GSE66749 platform GLP5175. The following samples were used: GSM1385160, GSM1385161, GSM1385162, GSM1385163, GSM1385164, GSM1385165, GSM1385166, GSM1385167, GSM1385168, GSM1385169, GSM1385170, GSM1385171, GSM1614646, GSM1614646. Sample values were determined using the GE02R “profile graph” function and searching for GATA4 and SATB2 by their ID numbers (U.S. Pat. Nos. 3,086,100 and 2,594,089 respectively).

RNA-seq sequence assembly abundance estimation. RNA library construction and RNA sequencing was performed by the Cincinnati Children's Hospital DNA Sequencing Core, using an Illumina HiSeq2500 platform. The quality of the Illumina sequencing run was evaluated by analyzing FASTQ data for each sample using FastQC version 0.10.1 http://www.bioinformatics.babraham.ac.uk/projects/fastqc to identify features of the data that may indicate quality problems (e.g. low-quality scores, over-represented sequences, inappropriate GC content, etc.). No major issues were identified by the QC analysis. Applicant used the software package Tuxedo Suite for alignment, differential expression analysis, and post-analysis diagnostics. Briefly, Applicant aligned reads to the reference transcriptome (UCSC hgl9) using TopHat version 2.0.13 and Bowtie version 2.2.5 (Langmead et al., 2009). Applicant used default parameter settings for alignment, with the exception of: “b2-very-sensitive” to maximize the accuracy of the read alignment, as well as “no-coverage-search” and “—no-no vel-juncs” limiting the read mapping to known transcripts. Cufflinks version 2.2.1 (Trapnell et al., 2012) was used for RNA abundance estimation. UCSC hgl9.fa was used as the reference genome sequence and UCSC hgl9.gtf was used for transcriptome annotation. Applicant applied the following parameters in Cufflinks: “—multi-read-correct” to adjust expression calculations for reads that map in more than one locus, and “—compatible-hits-norm” and “—upper-quartile—norm” for normalization of expression values. Normalized FPKM tables were generated using the CuffNorm function. RNA sequence assembly and transcriptional analysis was conducted using the 64-bit Debian Linux stable version 7.10 (“Wheezy”) platform.

Differential expression analysis.

All plots and statistical analyses were conducted in R version 3.3.1 (2016-06-21). Plots were generated using the R package ‘ggplot2’ (Ginestet, 2011). Differential expression analysis and statistical tests of Cufflinks output were completed with the R package ‘SeqRetriever’ ‘SeqRetriever’ version 0.6 https://github.com/hilldr/SeqRetrieyer. Hypergeometric means testing was used to evaluate relative enrichment of shared gene expression signatures between groups using the R package ‘GeneOverlap’ http://shenlab-sinai.cithub.io/shenlab-sinai/. The complete RNA-seq FASTQ processing pipeline and analysis scripts are available at https://qithub.com/hilldr/Munera2016.

### REFERENCES

Aronson, B. E., Aronson, S. R., Berkhout, R. P., Chavoushi, S. F., He, A., Pu, W. T., Verzi, M. P., and Krasinski, S. D. (2014). GATA4 represses an ileal program of gene expression in the proximal small intestine by inhibiting the acetylation of histone H3, lysine 27. Bba-Gene Regul Mech 1839, 1273-1282.
Battle, M. A., Bondow, B. J., Iverson, M. A., Adams, S. J., Jandacek, R. J., Tso, P., and Duncan, S. A. (2008). GATA4 is essential for jejuna! function in mice. Gastroenterology 135, 1676-I686 e1671.
Bernstein, B. E., Stamatoyannopoulos, J. A., Costello, J. F., Ren, B., Milosavljevic, A., Meissner, A., Kellis, M., Marra, M. A., Beaudet, A. L., Ecker, J. R., et al. (2010). The NIH Roadmap Epigenomics Mapping Consortium. Nat Biotechnol 28, 1045-1048.
Beuling, E., Bosse, T., aan de Kerk, D. J., Piaseckyj, C M., Fujiwara, Y., Katz, S. G., Orkin, S. H., Grand, R. J., and Krasinski, S. D. (2008a). GATA4 mediates gene repression in the mature mouse small intestine through interactions with friend of GATA (FOG) cofactors. Dev Biol 322, 179-189.
Beuling, E., Bosse, T., Buckner, M. A., and Krasinski, S. D. (2007a). Co-localization of Gata4 and Hnfl alpha in the gastrointestinal tract is restricted to the distal stomach and proximal small intestine. Gastroenterology 132, A586-A586.
Beuling, E., Bosse, T., de Kerk, D. A., Piaseckyj, C M., Fujiwara, Y., Orkin, S. H., and Krasinski, S. D. (2007b). Fog cofactors partially mediate Gata4 function in the adult mouse small intestine. Gastroenterology 132, A692-A693.
Beuling, E., Kerkhof, I. M., Nicksa, G. A., Giuffrida, M. J., Haywood, J., aan de Kerk, D. J., Piaseckyj, C M., Pu, W. T., Buchmiller, T. L., Dawson, P. A., et al. (2010). Conditional Gata4 deletion in mice induces bile acid absorption in the proximal small intestine. Gut 59, 888-895.
Beuling, E., Kerkhof, I. M., Piaseckyj, C M., Dawson, P A., Pu, W. T., Grand, R. J., and Krasinski, S. D. (2008b). The absence of GATA4 in the distal small intestine defines the ileal phenotype. Gastroenterology 134, A83-A84.
Bonilla-Claudio, M., Wang, J., Bai, Y., Klysik, E., Selever, J., and Martin, J. F. (2012). Bmp signaling regulates a dose-dependent transcriptional program to control facial skeletal development. Development 139, 709-719.
Bosse, T., Fialkovich, J. J., Piaseckyj, C M., Beuling, E., Broekman, H., Grand, R. J., Montgomery, R. K., and Krasinski, S. D. (2007). Gata4 and Hnflalpha are partially required for the expression of specific intestinal genes during development. Am J Physiol Gastrointest Liver Physiol 292, G1302-1314.
Bouchi, R., Foo, K. S., Hua, H., Tsuchiya, K., Ohmura, Y., Sandoval, P. R., Ratner, L. E., Egli, D., Leibel, R. L., and Accili, D. (2014). FOXOl inhibition yields functional insulin-producing cells in human gut organoid cultures. Nat Commun 5, 4242.
Burnicka-Turek, 0., Mohamed, B. A., Shirneshan, K., Thanasupawat, T., Hombach-Klonisch, S., Klonisch, T., and Adham, I. M. (2012). INSL5-deficient mice display an alteration in glucose homeostasis and an impaired fertility. Endocrinology 153, 4655-4665.
De Santa Barbara, P., Williams, J., Goldstein, A. M., Doyle, A. M., Nielsen, C, Winfield, S., Faure, S., and Roberts, D. J. (2005). Bone morphogenetic protein signaling pathway plays multiple roles during gastrointestinal tract development. Developmental dynamics. an official publication of the American Association of Anatomists 234, 312-322.
Dobreva, G., Chahrour, M., Dautzenberg, M., Chirivella, L., Kanzler, B., Farinas, I., Karsenty, G., and Grosschedl, R. (2006). SATB2 is a multifunctional determinant of craniofacial patterning and osteoblast differentiation. Cell 125, 971-986.
Driver, I., and Oh!stein, B. (2014). Specification of regional intestinal stem cell identity during Drosophila metamorphosis. Development 141, 1848-1856.
Duluc, I., Freund, J. N., Leberquier, C, and Kedinger, M. (1994). Fetal endoderm primarily holds the temporal and positional information required for mammalian intestinal development. J Cell Biol 126, 211-221.
Eberhard, J., Gaber, A., Wangefjord, S., Nodin, B., Uhlen, M., Ericson Lindquist, K., and Jirstrom, K. (2012). A cohort study of the prognostic and treatment predictive value of SATB2 expression in colorectal cancer. Br J Cancer 106, 931-938.
Fagerberg, L., Hallstrom, B. M., Oksvold, P., Kampf, C, Djureinovic, D., Odeberg, J., Habuka, M., Tahmasebpoor, S., Danielsson, A., Edlund, K., et al. (2014). Analysis of the human tissue-specific expression by genome-wide integration of transcriptomics and antibody-based proteomics. Mol Cell Proteomics 13, 397-406.
Finkbeiner, S. R., Hill, D. R., Altheim, C. H., Dedhia, P. H., Taylor, M. J., Tsai, Y. H., Chin, A. M., Mahe, M. M., Watson, C. L., Freeman, J. J., et al. (2015). Transcriptome-wide Analysis Reveals Hallmarks of Human Intestine Development and Maturation In vitro and In vivo. Stem Cell Reports.
FitzPatrick, D. R., Carr, I. M., McLaren, L., Leek, J. P., Wightman, P., Williamson, K., Gautier, P., McGill, N., Hayward, C, Firth, H., et al. (2003). Identification of SATB2 as the cleft palate gene on 2q32-q33. Hum Mol Genet 12, 2491-2501.
Georgas, K. M., Armstrong, J., Keast, J. R., Larkins, C. E., McHugh, K. M., Southard-Smith, E. M., Cohn, M. J., Batourina, E., Dan, H., Schneider, K., et a/. (2015). An illustrated anatomical ontology of the developing mouse lower urogenital tract. Development 142, 1893-1908.
Ginestet, C. (2011). ggplot2: Elegant Graphics for Data Analysis. J R Stat Soc a Stat 174, 245-245.
Gracz, A. D., Ramalingam, S., and Magness, S. T. (2010). Sox9 expression marks a subset of CD24-expressing small intestine epithelial stem cells that form organoids in vitro. Am J Physiol-Gastr L 298, G590-G600.
Guo, Z., Driver, I., and Ohlstein, B. (2013). Injury-induced BMP signaling negatively regulates Drosophila midgut homeostasis. J Cell Biol 201, 945-961.
Gyorgy, A. B., Szemes, M., de Juan Romero, C, Tarabykin, V., and Agoston, D. V. (2008). SATB2 interacts with chromatin-remodeling molecules in differentiating cortical neurons. Eur J Neurosci 27, 865-873.
Haramis, A. P. G., Begthel, H., van den Born, M., van Es, J., Jonkheer, S., Offerhaus, G. J. A., and Clevers, H. (2004). De novo crypt formation and juvenile polyposis on BMP inhibition in mouse intestine. Science 303, 1684-1686.
Hardwick, J. C., Van Den Brink, G. R., Bleuming, S. A., Ballester, I., Van Den Brande, J. M., Keller, J. J., Offerhaus, G. J., Van Deventer, S. J., and Peppelenbosch, M. P. (2004). Bone morphogenetic protein 2 is expressed by, and acts upon, mature epithelial cells in the colon. Gastroenterology 126, 111-121.
He, X. C., Zhang, J. W., Tong, W. G., Tawfik, 0., Ross, J., Scoville, D. H., Tian, Q., Zeng, X., He, X., Wiedemann, L. M., et a/. (2004). BMP signaling inhibits intestinal stem cell self-renewal through suppression of Wnt-beta-catenin signaling. Nature Genetics 36, 1117-1121.
Higuchi, Y., Kojima, M., Ishii, G., Aoyagi, K., Sasaki, H., and Ochiai, A. (2015). Gastrointestinal Fibroblasts Have Specialized, Diverse Transcriptional Phenotypes: A Comprehensive Gene Expression Analysis of Human Fibroblasts. Plos One 10.
Holland, P. W. H., Booth, H. A. F., and Bruford, E. A. (2007). Classification and nomenclature of all human homeobox genes. Bmc Biol 5.
Jeejeebhoy, K. N. (2002). Short bowel syndrome: a nutritional and medical approach. CMAJ 166, 1297-1302.
Johnston, T. B. (1913). Extroversion of the Bladder, complicated by the Presence of Intestinal Openings on the Surface of the Extroverted Area. J Anat Physiol 48, 89-106.
Kohlnhofer, B. M., Thompson, C. A., Walker, E. M., and Battle, M. A. (2016). GATA4 regulates epithelial cell proliferation to control intestinal growth and development in mice. Cell Mol Gastroenterol Hepatol 2, 189-209.
Kumar, M., Jordan, N., Melton, D., and Grapin-Botton, A. (2003). Signals from lateral plate mesoderm instruct endoderm toward a pancreatic fate. Dev Biol 259, 109-122.
Langmead, B., Trapnell, C, Pop, M., and Salzberg, S. L. (2009). Ultrafast and memory-efficient alignment of short DNA sequences to the human genome. Genome Biol 10, R25.
Li, H., Coghlan, A., Ruan, J., Coin, L. J., Heriche, J. K., Osmotherly, L., Li, R., Liu, T., Zhang, Z., Bolund, L., et al. (2006). TreeFam: a curated database of phylogenetic trees of animal gene families. Nucleic Acids Res 34, D572-580.
Li, L. H. (2005). BMP signaling inhibits intestinal stem cell self-renewal through antagonizing Wnt signaling. Gastroenterology 128, A702-A702.
McCracken, K. W., Aihara, E., Martin, B., Crawford, C M., Broda, T., Treguier, J., Zhang, X., Shannon, J. M., Montrose, M. H., and Wells, J. M. (2017). Wnt/beta-catenin promotes gastric fundus specification in mice and humans. Nature 541, 182-187.
McCracken, K. W., Cata, E. M., Crawford, C M., Sinagoga, K. L., Schumacher, M., Rockich, B. E., Tsai, Y. H., Mayhew, C. N., Spence, J. R., Zavros, Y., et al. (2014). Modelling human development and disease in pluripotent stem-cell-derived gastric organoids. Nature 516, 400-404.
McGovern, D. P., Gardet, A., Torkvist, L., Goyette, P., Essers, J., Taylor, K. D., Neale, B. M., Ong, R. T., Lagace, C, Li, C, et al. (2010). Genome-wide association identifies multiple ulcerative colitis susceptibility loci. Nat Genet 42, 332-337.
Molodecky, N A., Soon, I. S., Rabi, D. M., Ghali, W. A., Ferris, M., Chernoff, G., Benchimol, E. I., Panaccione, R., Ghosh, S., Barkema, H. W., et al. (2012). Increasing incidence and prevalence of the inflammatory bowel diseases with time, based on systematic review. Gastroenterology 142, 46-54 e42; quiz e30.
Moser, A. R., Pitot, H. C, and Dove, W. F. (1990). A Dominant Mutation That Predisposes to Multiple Intestinal Neoplasia in the Mouse. Science 247, 322-324.
Patankar, J., Obrowsky, S., Hoefler, G., Battle, M., Kratky, D., and Levak-Frank, S. (2012a). Intestinal Deficiency of Gata4 Protects from Diet-Induced Hepatic Steatosis by Suppressing De Novo Lipogenesis and Gluconeogenesis in Mice. J Hepatol 56, S496-S496.
Patankar, J. V., Obrowsky, S., Doddapattar, P., Hoefler, G., Battle, M., Levak-Frank, S., and Kratky, D. (2012b). Intestinal GATA4 deficiency protects from diet-induced hepatic steatosis. J Hepatol 57, 1061-1068.
Ramalingam, S., Daughtridge, G. W., Johnston, M. J., Gracz, A. D., and Magness, S. T. (2012). Distinct levels of Sox9 expression mark colon epithelial stem cells that form colonoids in culture. Am J Physiol Gastrointest Liver Physiol 302, G10-20.
Rankin, S. A., Gallas, A. L., Neto, A., Gomez-Skarmeta, J. L., and Zorn, A. M. (2012). Suppression of Bmp4 signaling by the zinc-finger repressors Osr1 and Osr2 is required for Wnt/beta-catenin-mediated lung specification in Xenopus. Development 139, 3010-3020.
Rankin, S. A., Thi Tran, H., Wlizla, M., Mancini, P., Shifley, E. T., Bloor, S. D., Han, L., Vleminckx, K., Wert, S. E., and Zorn, A. M. (2015). A Molecular atlas of Xenopus respiratory system development. Developmental dynamics. an official publication of the American Association of Anatomists 244, 69-85.
Ratineau, C, Duluc, I., Pourreyron, C, Kedinger, M., Freund, J. N., and Roche, C. (2003). Endoderm- and mesenchyme-dependent commitment of the differentiated epithelial cell types in the developing intestine of rat. Differentiation 71, 163-169.
Roberts, D. J., Johnson, R. L., Burke, A. C., Nelson, C. E., Morgan, B. A., and Tabin, C. (1995). Sonic Hedgehog Is an Endodermal Signal Inducing Bmp-4 and Hox Genes during Induction and Regionalization of the Chick Hindgut. Development 121, 3163-3174.
Rodriguez-Pineiro, A. M., Bergstrom, J. H., Ermund, A., Gustafsson, J. K., Schutte, A., Johansson, M. E., and Hansson, G. C. (2013). Studies of mucus in mouse stomach, small intestine, and colon. II. Gastrointestinal mucus proteome reveals Muc2 and Muc5ac accompanied by a set of core proteins. Am J Physiol Gastrointest Liver Physiol 305, G348-356.
Savidge, T. C., Morey, A. L., Ferguson, D. J., Fleming, K. A., Shmakov, A. N., and Phillips, A. D. (1995). Human intestinal development in a severe-combined immunodeficient xenograft model. Differentiation 58, 361-371.
Savin, T., Kurpios, N. A., Shyer, A. E., Florescu, P., Liang, H., Mahadevan, L., and Tabin, CJ. (2011). On the growth and form of the gut. Nature 476, 57-62.
Sheehan-Rooney, K., Swartz, M. E., Lovely, C. B., Dixon, M. J., and Eberhart, J. K. (2013). Bmp and Shh signaling mediate the expression of satb2 in the pharyngeal arches. PLoS One 8, e59533.
Sherwood, R. I., Chen, T. Y., and Melton, D. A. (2009). Transcriptional dynamics of endodermal organ formation. Developmental dynamics. an official publication of the American Association of Anatomists 238, 29-42.
Sherwood, R. I., Maehr, R., Mazzoni, E. O., and Melton, D. A. (2011). Wnt signaling specifies and patterns intestinal endoderm. Mech Dev 128, 387-400.
Shyer, A. E., Huycke, T. R., Lee, C, Mahadevan, L., and Tabin, CJ. (2015). Bending gradients: how the intestinal stem cell gets its home. Cell 161, 569-580.
Siegel, R., Desantis, C, and Jemal, A. (2014). Colorectal cancer statistics, 2014. CA Cancer J Clin 64, 104-117.
Spence, J. R., Mayhew, C. N., Rankin, S. A., Kuhar, M. F., Valiance, J. E., Tolle, K., Hoskins, E. E., Kalinichenko, V. V., Wells, Si., Zorn, A. M., et al. (2011). Directed differentiation of human pluripotent stem cells into intestinal tissue in vitro. Nature 470, 105-109.
Thanasupawat, T., Harnmje, K., Adham, I., Ghia, J. E., Del Bigio, M. R., Krcek, J., Hoang-Vu, C, Klonisch, T., and Hombach-Klonisch, S. (2013). INSL5 is a novel marker for human enteroendocrine cells of the large intestine and neuroendocrine tumours. Oncol Rep 29, 149-I54.
Tiso, N., Filippi, A., Pauls, S., Bortolussi, M., and Argenton, F. (2002). BMP signalling regulates anteroposterior endoderm patterning in zebrafish. Mech Dev 118, 29-37.
Trapnell, C, Roberts, A., Goff, L., Pertea, G., Kim, D., Kelley, D. R., Pimentel, H., Salzberg, S. L., Rinn, J. L., and Pachter, L. (2012). Differential gene and transcript expression analysis of RNA-seq experiments with TopHat and Cufflinks. Nat Protoc 7, 562-578.
Uppal, K., Tubbs, R. S., Matusz, P., Shaffer, K., and Loukas, M. (2011). Meckel's diverticulum: a review. Clin Anat 24, 416-422.
van Dop, W. A., Uhmann, A., Wijgerde, M., Sleddens-Linkels, E., Heijmans, J., Offerhaus, G. J., Weerman, M. A. V., Boeckxstaens, G. E., Hommes, D. W., Hardwick, J. C., et al. (2009). Depletion of the Colonic Epithelial Precursor Cell Compartment Upon Conditional Activation of the Hedgehog Pathway. Gastroenterology 136, 2195-2203.
van Klinken, B. J., Dekker, J., van Gool, S. A., van Marie, J., Buller, H. A., and Einerhand, A. W. (1998). MUCSB is the prominent mucin in human gallbladder and is also expressed in a subset of colonic goblet cells. The American journal of physiology 274, G871-878.
Walker, E. M., Thompson, C. A., and Battle, M. A. (2014). GATA4 and GATA6 regulate intestinal epithelial cytodifferentiation during development. Dev Biol 392, 283-294.
Walton, K. D., Kolterud, A., Czerwinski, M. J., Bell, M. J., Prakash, A., Kushwaha, J., Grosse, A. S., Schnell, S., and Gumucio, D. L. (2012). Hedgehog-responsive mesenchymal clusters direct patterning and emergence of intestinal villi. Proc Natl Acad Sci USA 109, 15817-15822.
Walton, K. D., Kolterud, A., Grosse, A. S., Hu, C. B., Czerwinski, M., Richards, N., and Gumucio, D. L. (2009). Epithelial Hedgehog signals direct mesenchymal villus patterning through BMP. Dev Biol 331, 489-489.
Walton, K. D., Whidden, M., Kolterud, A., Shoffner, S. K., Czerwinski, M. J., Kushwaha, J., Parmar, N., Chandhrasekhar, D., Freddo, A. M., Schnell, S., et al. (2016). Villification in the mouse: Bmp signals control intestinal villus patterning. Development 143, 427-436.
Wang, X., Yamamoto, Y., Wilson, L. H., Zhang, T., Howitt, B. E., Farrow, M. A., Kern, F., Ning, G., Hong, Y., Khor, C. C., etal. (2015). Cloning and variation of ground state intestinal stem cells. Nature 522, 173-178.
Watson, C. L., Mahe, M. M., Munera, J., Howell, J. C., Sundaram, N., Poling, H. M., Schweitzer, J. I., Valiance, J. E., Mayhew, C. N., Sun, Y., et al. (2014). An in vivo model of human small intestine using pluripotent stem cells. Nat Med 20, 1310-1314.
Wehkamp, J., Chu, H., Shen, B., Feathers, R. W., Kays, R. J., Lee, S. K., and Bevins, C. L. (2006). Paneth cell antimicrobial peptides: topographical distribution and quantification in human gastrointestinal tissues. FEBS Left 580, 5344-5350.
Whissell, G., Montagni, E., Martinelli, P., Hernando-Momblona, X., Sevillano, M., Jung, P., Cortina, C, Caton, A., Abuli, A., Castells, A., et al. (2014). The transcription factor GATA6 enables self-renewal of colon adenoma stem cells by repressing BMP gene expression. Nat Cell Biol 16, 695-707.
Wills, A., Dickinson, K., Khokha, M., and Baker, J. C. (2008). Bmp signaling is necessary and sufficient for ventrolateral endoderm specification in Xenopus. Developmental dynamics: an official publication of the American Association of Anatomists 237, 2177-2186.
Xue, X., Ramakrishnan, S., Anderson, E., Taylor, M., Zimmermann, E. M., Spence, J. R., Huang, S., Greenson, J. K., and Shah, Y. M. (2013). Endothelial PAS domain protein 1 activates the inflammatory response in the intestinal epithelium to promote colitis in mice. Gastroenterology 145, 831-841.
Yahagi, N., Kosaki, R., Ito, T., Mitsuhashi, T., Shimada, H., Tomita, M., Takahashi, T., and Kosaki, K. (2004). Position-specific expression of Hox genes along the gastrointestinal tract. Congenit Anom (Kyoto) 44, 18-26.
Zbuk, K. M., and Eng, C. (2007). Hamartomatous polyposis syndromes. Nat Clin Pract Gastr 4, 492-502.
Zorn, A. M., and Wells, J. M. (2009). Vertebrate endoderm development and organ formation. Annu Rev Cell Dev Biol 25, 221-251.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of inducing formation of a human colon organoid (HCO), comprising the steps of
a. contacting definitive endoderm (DE) with an FGF signaling pathway activator and a WNT signaling pathway activator for a period of time sufficient for said DE to form a mid-hindgut spheroid;
b. contacting the mid-hindgut spheroid of step (a) with a BMP activator and an EGF signaling pathway activator for a period of time sufficient to form said human colon organoid, wherein said human colon organoid expresses SATB2.

2. The method of claim 1 wherein said DE is derived from a precursor cell selected from an embryonic stem cell, and an induced pluripotent stem cell.

3. The method of claim 1, wherein said FGF signaling pathway activator is selected from a small molecule FGF signaling pathway activator, a protein-based FGF signaling pathway activator, FGF1, FGF2, FGF3, FGF4, FGF10, FGF11, FGF12, FGF13, FGF14, FGF15, FGF16, FGF17, FGF18, FGF19, FGF20, FGF21, FGF22, FGF23, or combinations thereof.

4. The method of claim 1, wherein said WNT signaling pathway activator is selected from a protein Wnt signaling pathway activator, a small molecule Wnt signaling pathway activator, Lithium Chloride; 2-amino-4,6-disubstituted pyrimidine (hetero) arylpyrimidines; IQ1; QS11; NSC668036; DCA beta-catenin; 2-amino-4-[3,4-(methylenedioxy)-benzyl-amino]-6-(3-methoxyphenyl) pyrimidine, Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, a GSK3 inhibitor, CHIR99021, or combinations thereof.

5. The method of claim 1, wherein said BMP activator is selected from BMP2, BMP4, BMP7, BMP9, a small molecule that activates the BMP pathway, a protein that activate the BMP pathway, ventromorphins, and combinations thereof.

6. The method of claim 1, wherein said period of time sufficient for said DE to form a mid-hindgut spheroid is determined by expression of CDX2 by said mid-hindgut spheroid of step (a).

7. The method of claim 1, wherein said period of time sufficient for said mid-hindgut spheroid to form said human colon organoid by expression of SATB2 and CDX2 by a cell of said human colon organoid.

8. The method of claim 1, wherein said HCO is characterized by the presence of colonic enteroendocrine cells (EEC).

9. The method of claim 1, wherein said HCO is characterized by the presence of crypts and is substantially free of villi.

10. The method of claim 1, wherein said HCO comprises colon-specific goblet cells.

11. The method of claim 1, wherein said HCO is substantially free of Paneth cells.

12. The method of claim 1, wherein said HCO secretes colon-specific hormone INSL5.

13. An HCO obtained according to the method of claim 1.

14. A method of forming colonic tissue, comprising engrafting the HCO of claim 13, under a kidney capsule of a mammal.

15. A method of determining the efficacy and/or toxicity of a potential therapeutic agent for a disease selected from colitis, colon cancer, polyposis syndromes, and/or irritable bowel syndrome, comprising contacting said potential therapeutic agent with the HCO of claim 13 for a period of time sufficient to determine the efficacy and/or toxicity of said potential therapeutic agent.

16. An immunocompromised rodent comprising the HCO of claim 13.

17. An intestinal colonoid derived from the HCO of claim 13.

18. The intestinal colonoid of claim 17, wherein said intestinal colonoid is free of one or more of an immune function, innervation, blood vessels, villi, and Paneth cells.
